(19) United States
(12) Patent Application Publication
(10) Pub. No.: US 2024/0262819 A1
(43) Pub. Date: Aug. 8, 2024

(54) NOVEL COMPOUNDS HAVING INHIBITORY ACTIVITY AGAINST GLUCOSYLCERAMIDE SYNTHASE OR PHARMACEUTICALLY ACCEPTABLE SALT THEREOF, PROCESSES FOR PREPARING THE SAME, AND PHARMACEUTICAL COMPOSITIONS COMPRISING THE SAME

(71) Applicants: YUHAN CORPORATION, Seoul (KR); GREEN CROSS CORPORATION, Yongin-si, Gyeonggi-do (KR)

(72) Inventors: Dong-Hoon KIM, Suwon-si, Gyeonggi-do (KR); Jae-Eun JOO, Yongin-si, Gyeonggi-do (KR); Seung-Yub SHIN, Seongnam-si, Gyeonggi-do (KR); Sool-Ki KWON, Yongin-si, Gyeonggi-do (KR); Jong-Suk PARK, Anyang-si, Gyeonggi-do (KR)

(73) Assignees: YUHAN CORPORATION, Seoul (KR); GREEN CROSS CORPORATION, Yongin-si, Gyeonggi-do (KR)

(21) Appl. No.: 18/559,466

(22) PCT Filed: May 10, 2022
(86) PCT No.: PCT/KR2022/006613

§ 371 Date: Nov. 7, 2023

(30) Foreign Application Priority Data
May 11, 2021 (KR) 10-2021-0060942

(51) Int. Cl. C07D 453/02; A61K 31/439

(52) U.S. Cl. CPC C07D 453/02; A61K 31/439

(57) ABSTRACT

Provided are a compound having an inhibitory activity against glucosylceramide synthase (GCS), i.e., compounds having a 2,3-dihydro-1H-indene, 1,2,3,4-tetrahydronaphthalene, or chromane moiety, or pharmaceutically acceptable salt thereof, a process for the preparation thereof, a pharmaceutical composition comprising the same and a use thereof, wherein the compound or pharmaceutically acceptable salt thereof has an inhibitory activity against glucosylceramide synthase (GCS), and exhibits the effects of alleviating symptoms in the central nervous system as well as in the peripheral nervous system, through excellent blood-brain barrier permeability, and therefore, the compound or pharmaceutically acceptable salt thereof can be usefully applied for preventing or treating various diseases associated with GCS, such as Gaucher disease, Fabry disease, Tay-Sachs disease, Parkinson's disease, etc.

### TECHNICAL FIELD

The present invention relates to a novel compound having an inhibitory activity against glucosylceramide synthase (GCS) or pharmaceutically acceptable salt thereof, a process for the preparation thereof, a pharmaceutical composition comprising the same and a use thereof.

### BACKGROUND ART

Lysosomal storage disorders (LSDs) are the metabolic disorders that result from genetic lack or deficiency of certain enzymes in lysosomes. LSDs exhibit various pathological symptoms throughout the body, as the non-metabolized or non-degraded substrates are accumulated. Currently, about 50 types of LSDs are known and are largely classified into the diseases such as mucopolysaccharidoses, oligosaccharidoses, and sphingolipidoses, depending on the substances accumulated.

Among the diseases, sphingolipidoses, which are a class of glycolipid storage disorders relating to sphingolipid metabolism, show pathologies due to the accumulation of various membrane glycosphingolipids (GSLs), such as glucosylceramide, trihexocylceramide, etc. For example, when the enzymes related to sphingolipid metabolism, such as beta-glucosidase, alpha-galactosidase, etc., do not have normal activity, the substrates thereof (e.g., glucosylceramide, trihexocylceramide, etc.) are accumulated, thereby exhibiting various pathologies, such as Gaucher disease, Fabry disease, etc.

20)

There are known two types of therapies for the LSDs. The first method is to replace or supplement the insufficient or deficient metabolic enzymes. Although such an enzyme replacement therapy (ERT) is safe and effective, periodic intravenous administrations of the related enzyme(s) are required; the dose thereof should be adjusted according to the enzyme reaction(s); and the costs thereof are relatively high. Especially, since it is difficult to distribute the enzyme(s) toward the nervous system, the ERT does not show satisfactory efficacy in the treatment of symptoms related to the nervous system. In addition, there is also the problem that autoantibodies against the administered enzyme(s) are frequently generated.

The second method is a substrate reduction therapy (SRT) for inhibiting the syntheses of accumulated substrates. Glucosylceramide synthase (GCS) (also referred to as “UDP-glucose: ceramide “UDP-glucose: N-acylsphingosine glycosyltransferase”, D-glucosyltransferase“, or “EC 2.4.1.80”), which is an enzyme involved in sphingolipid metabolism, is involved in the reaction of ceramide with glucose to produce glucosylceramide.

The resulting glucosylceramide is converted into various GSLs. Glucosylceramide synthase (GCS) inhibitors inhibit the activity of GCS, thereby reducing the production of glucosylceramide in the body and preventing abnormal accumulation of glycolipids, such as trihexocylceramide, GM1, and GM2, in cells or organs.

As such, GCS inhibitors inhibit the activity of GCS to prevent the accumulation of glycolipids and thus may be usefully applied to the treatment of lysosomal storage disorders, especially glycolipid storage disorders, such as GM1 gangliosidosis, Tay-Sachs disease, Sandhoff disease, Gaucher disease, Fabry disease, Niemann-Pick disease (types A and B), metachromatic leukodystrophy, Krabbe disease, etc. GCS inhibitors may be also used in the treatment of the secondary diseases associated with glycolipid storage, such as Niemann-Pick disease (type C), mucopolysaccharidosis, and mucolipidosis type IV (see, Chen C S, et al., Abnormal transport along the lysosomal pathway in mucolipidosis, type IV disease Proc Natl Acad Sci USA. 1998 May 26;95(11):6373-8; and Goodman L A, et al., Ectopic dendrites occur only on cortical pyramidal cells containing elevated GM2 ganglioside in alpha-mannosidosis, Proc Natl Acad Sci USA. 1991 Dec. 15; 88(24):11330-4). In addition, it has been reported to be useful in the treatment of diseases associated with the accumulation of glycolipids, such as renal hypertrophy (e.g., diabetic kidney disease); hyperglycemia or hyperinsulinemia; cancers with abnormal glycolipid synthesis; infectious diseases caused by the organisms using cell-surface glycolipids as a receptor; infectious diseases where the synthesis of glucosylceramide is essential or important; diseases in which excessive glycolipid synthesis occurs (e.g., atherosclerosis, polycystic kidney disease, and renal hypertrophy); neurological disorders and/or damages associated with the replenishment and activity of macrophages (e.g., Alzheimer's disease, epilepsy, stroke, spinal cord diseases, Parkinson's disease, etc.); inflammatory diseases or disorders (e.g., rheumatoid arthritis, Crohn's disease, asthma, sepsis); and diabetes and obesity (see, WO 2006/053043). And, overexpression of GCS interferes with ceramide-induced apoptosis (see, Liu Y Y, et al., Uncoupling ceramide glycosylation by transfection of glucosylceramide synthase antisense reverses adriamycin resistance, J Biol Chem. 2000 Mar. 10; 275(10):7138-43). Therefore, GCS inhibitors may be useful for treating proliferative diseases, such as cancer, by inducing apoptosis in diseased cells.

Various studies have been conducted to develop GCS inhibitors. For example, various compounds having an inhibitory activity against GCS have been disclosed in WO 2005/068426, WO 2006/053043, WO 2008/150486, WO 2009/117150, WO 2010/014554, WO 2014/043068, etc.

### DISCLOSURE

### Technical Problem

The present inventors have found that a novel compound having a 2,3-dihydro-1H-indene, 1,2,3,4-tetrahydronaphthalene, or chromane moiety or pharmaceutically acceptable salt thereof not only has an excellent inhibitory activity against glucosylceramide synthase (GCS) but also exhibits excellent blood-brain barrier permeability. Therefore, said compound or pharmaceutically acceptable salt thereof can be usefully applied for preventing or treating various diseases associated with GCS, such as Gaucher disease, Fabry disease, Tay-Sachs disease, Parkinson's disease, etc.

Therefore, the present invention provides the above derivative having a 2,3-dihydro-1H-indene, 1,2,3,4-tetrahydronaphthalene, or chromane moiety or pharmaceutically acceptable salt thereof, a process for the preparation thereof, a pharmaceutical composition comprising the same, and a use thereof.

### Technical Solution

According to an aspect of the present invention, there is provided a derivative having a 2,3-dihydro-1H-indene, 1,2,3,4-tetrahydronaphthalene, or chromane moiety or pharmaceutically acceptable salt thereof.

According to another aspect of the present invention, there is provided a process for preparing said derivative having a 2,3-dihydro-1H-indene, 1,2,3,4-tetrahydronaphthalene, or chromane moiety or pharmaceutically acceptable salt thereof.

According to still another aspect of the present invention, there is provided a pharmaceutical composition comprising said derivative having a 2,3-dihydro-1H-indene, 1,2,3,4-tetrahydronaphthalene, or chromane moiety or pharmaceutically acceptable salt thereof as an active ingredient.

According to still another aspect of the present invention, there is provided a therapeutic method comprising administering said derivative having a 2,3-dihydro-1H-indene, 1,2,3,4-tetrahydronaphthalene, or chromane moiety or pharmaceutically acceptable salt thereof.

According to still another aspect of the present invention, there is provided a use of said derivative having a 2,3-dihydro-1H-indene, 1,2,3,4-tetrahydronaphthalene, or chromane moiety or pharmaceutically acceptable salt thereof for the manufacture of a medicament for inhibiting glucosylceramide synthase.

### Advantageous Effects

It has been found by the present invention that the derivative having a 2,3-dihydro-1H-indene, 1,2,3,4-tetrahydronaphthalene, or chromane moiety or pharmaceutically acceptable salt thereof not only has an excellent inhibitory activity against glucosylceramide synthase (GCS) but also exhibits the effects of alleviating symptoms in the central nervous system as well as in the peripheral nervous system, through excellent blood-brain barrier permeability.

Therefore, the compound or pharmaceutically acceptable salt thereof according to the present invention can be usefully applied for preventing or treating various diseases associated with GCS, such as Gaucher disease, Fabry disease, Tay-Sachs disease, Parkinson's disease, etc.

### DESCRIPTION OF DRAWINGS

FIG. 1 shows a single-crystal X-ray structure of the compound prepared in Preparation 1.

FIG. 2 shows a packing crystal structure, showing the view along the a-axis, of the compound prepared in Preparation 1.

FIG. 3 shows a packing crystal structure, showing the view along the b-axis, of the compound prepared in Preparation 1.

FIG. 4 shows a packing crystal structure, showing the view along the c-axis, of the compound prepared in Preparation 1.

### BEST MODE FOR CARRYING OUT THE INVENTION

As used herein, the term “alkyl” refers to a straight or branched aliphatic hydrocarbon radical. For example, the C₁˜C₆ alkyl means a straight or branched aliphatic hydrocarbon having 1 to 6 carbon atoms, such as methyl, ethyl, propyl, n-butyl, n-pentyl, n-hexyl, isopropyl, isobutyl, see-butyl, tert-butyl, neopentyl, and isopentyl.

20) The term “hydroxy” refers to the ‘—OH’ group. The term “alkoxy” refers to a radical formed by substituting the hydrogen atom in the hydroxyl group with an alkyl. For example, the C₁˜C₆ alkoxy includes methoxy, ethoxy, propoxy, n-butoxy, n-pentyloxy, isopropoxy, see-butoxy, tert-butoxy, neopentyloxy, and isopentyloxy.

The term “halogen” refers to the fluoro, bromo, chloro, or iodo group.

25 The term “cycloalkyl” refers to a saturated aliphatic 3— to 10-membered ring, preferably 3— to 7-membered ring, unless otherwise defined. Typical cycloalkyl groups include cyclopropyl, cyclobutyl, cyclopentyl, cyclohexyl, and the like, but are not limited thereto.

The term “aryl” refers to an organic radical derived from an aromatic hydrocarbon, through removing one hydrogen atom therefrom, including mono or poly-fused ring systems such as 5— to 14-membered substituted or unsubstituted rings and a form in which a plurality of aryls are connected by a single bond. The “aryl” includes, for example, phenyl, naphthyl, biphenyl, terphenyl, anthryl, indenyl, fluorenyl, phenanthryl, triphenylenyl, pyrenyl, perylenyl, chrysenyl, naphthacenyl, fluoranthenyl, and the like, but are not limited thereto.

The term “heteroaryl” refers to a 5— to 12-membered aromatic radical having one to three heteroatoms selected from the group consisting of nitrogen (N) atom, oxygen (O) atom, and sulfur (S) atom, including a 5— or 6-membered monocyclic heteroaryl radical and a bicyclic heteroaryl radical formed by fusing the 5— or 6-membered monocyclic heteroaryl radical with a benzene or pyridine ring. And, the term “heterocycle” refers to a 3— to 12-membered mono—or poly-cyclic ring having one or more, preferably one to four, same or different heteroatoms selected from oxygen (O) atom, nitrogen (N) atom, and sulfur (S) atom, but not containing an aromatic ring. Non-limiting examples of heteroaryl or heterocyclic rings include oxetane, pyrrolidine, pyrrole, tetrahydrofuran, furan, tetrahydrothiophene, thiophene, imidazolidine, imidazole, pyrazolidine, pyrazole, pyrrolizine, oxazolidine, oxazole, isoxazolidine, isoxazole, thiazolidine, thiazole, isothiazolidine, isothiazole, dioxolane, dithiolane, oxadiazole, thiadiazole, dithiazole, tetrazole, oxatetrazole, thiatetrazole, piperidine, pyridine, pyrimidine, tetrahydropyran, pyran, thiane, thiopyran, piperazine, diazine, morpholine, oxazine, dioxane, indole, indoline, benzodioxole, benzothiophene, benzofuran, benzimidazole, bezoxazole, benzisoxazole, benzothiazole, benzothiadiazole, benzotriazole, quinoline, isoquinoline, purine, furopyridine, mono- or di-azabicycles (such as quinuclidine, diazabicycloheptane, monoazabicyclooctane, diazaspiroundecane, etc.), hexahydropyrrolopyrrole, pyrrolopyrrole, pyrrolopyridine, imidazopyridazine, dihydrobenzodioxine, dihydrobenzofuran, and the like, but are not limited thereto.

The term “amino” refers to the ‘—NH₂’ group. The term “alkylamino” refers to an amino group substituted with mono- or di-alkyl. For example, the C₁˜C₆ alkylamino group includes an amino group substituted with mono- or di-C₁˜C₆ alkyl group.

The term “alkylthio” refers to the ‘—SR’ group, in which R is an alkyl. The term “cyano” refers to the ‘—CN’.

The present invention provides a derivative having a 2,3-dihydro-1H-indene, 1,2,3,4-tetrahydronaphthalene, or chromane moiety or pharmaceutically acceptable salt thereof, that is a compound of Formula 1 or pharmaceutically acceptable salt thereof:

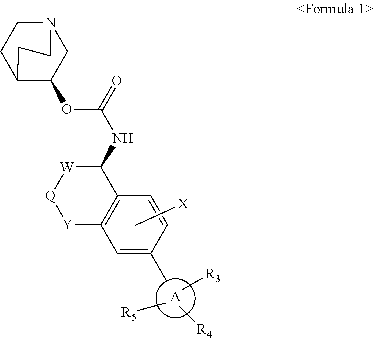

wherein,
W and Q are, independently each other, —CR₁R₂—,
Y is a bond, —CR₁′R₂′—; or —O—,
R₁ and R₂ are, independently each other, hydrogen; halogen; C₁˜C₆ alkyl; C₁˜C₆ alkyl having a nitrogen, oxygen, or sulfur atom; C₃˜C₁₀ cycloalkyl; 3— to 12-membered heterocyclic; or C₁˜C₆ alkoxy; or R₁ and R₂ form C₃˜C₁₀ cycloalkyl together with the carbon atom to which they are attached,
R₁′ and R₂′ are, independently each other, hydrogen; halogen; C₁˜C₆ alkyl; C₁˜C₆ alkyl having a nitrogen, oxygen, or sulfur atom; C₃˜C₁₀ cycloalkyl; 3— to 12-membered heterocyclic; or C₁˜C₆ alkoxy; or R₁ and R₂ form C₃˜C₁₀ cycloalkyl together with the carbon atom to which they are attached,
X is hydrogen; halogen; C₁˜C₆ alkyl; C₁˜C₆ alkyl substituted with 1 to 3 halogens; C₁˜C₆ alkyl having a nitrogen, oxygen, or sulfur atom; C₁˜C₆ alkoxy; or C₁˜C₆ alkoxy substituted with 1 to 3 halogens,
A ring is 6— to 12-membered aryl; 5— to 12-membered heteroaryl; C₃˜C₁₀ cycloalkyl; or 3— to 12-membered heterocyclic, and
R₃, R₄, and R₅ are, independently each other, hydrogen; cyano; halogen; C₁˜C₆ alkyl; C₁˜C₆ alkoxy-C₁˜C₆ alkyl; C₁˜C₆ alkyl substituted with 1 to 3 halogens; C₃˜C₁₀ cycloalkyl; 3—to 12-membered heterocyclic; C₁˜C₆ alkoxy; C₁˜C₆ alkoxy substituted with 1 to 3 halogens; C₁˜C₆ alkoxy-C₁˜C₆ alkoxy; morpholinyl-C₁˜C₆ alkoxy; mono- or di-C₁˜C₆ alkylamino-C₁˜C₆ alkoxy; C₃˜C₁₀ cycloalkyl-C₁˜C₆ alkoxy; C₁˜C₆ alkylthio; amino; mono- or di-C₁˜C₆ alkylamino; C₁˜C₆ alkylcarbonyl; hydroxy; or nitro.

In the compound of Formula 1 or pharmaceutically acceptable salt thereof according to the present invention, W may be —C(CH₃)₂—.

In the compound of Formula 1 or pharmaceutically acceptable salt thereof according to the present invention, Y may be a bond or —CH₂—.

In the compound of Formula 1 or pharmaceutically acceptable salt thereof according to the present invention, Y may be a bond (i.e., Y may form a 2,3-dihydro-1H-indene moiety together with W).

In the compound of Formula 1 or pharmaceutically acceptable salt thereof according to the present invention, Y may be —CH₂— (i.e., Y may form a 1,2,3,4-tetrahydronaphthalene moiety together with W).

In the compound of Formula 1 or pharmaceutically acceptable salt thereof according to the present invention, Y may be —O— (i.e., Y may form a chromane moiety together with W). In the compound of Formula 1 or pharmaceutically acceptable salt thereof according to the present invention, X may be hydrogen, halogen, C₁˜C₆ alkyl, or C₁˜C₆ alkoxy.

In the compound of Formula 1 or pharmaceutically acceptable salt thereof according to the present invention, the A ring may be phenyl or pyridinyl.

In the compound of Formula 1 or pharmaceutically acceptable salt thereof according to the present invention, R₃, R₄ and R₅ may be, independently each other, hydrogen, halogen, C₁˜C₆ alkyl, C₁˜C₆ alkoxy, C₁˜C₆ alkoxy substituted with 1 to 3 halogens, or C₃˜C₁₀ cycloalkyl-C₁˜C₆ alkoxy.

In an embodiment of the present invention,
W is —C(CH₃)₂—,
Q is —CH₂—,
Y is a bond,
X is hydrogen, halogen, C₁˜C₆ alkyl, or C₁˜C₆ alkoxy,
the A ring is phenyl or pyridinyl, and
R₃, R₄ and R₅ are, independently each other, hydrogen, halogen, C₁˜C₆ alkyl, C₁˜C₆ alkoxy, C₁˜C₆ alkoxy substituted with 1 to 3 halogens, or C₃˜C₁₀ cycloalkyl-C₁˜C₆ alkoxy.

In another embodiment of the present invention,
W is —C(CH₃)₂—,
Q is —CH₂—,
Y is —CH₂—,
X is hydrogen, halogen, C₁˜C₆ alkyl, or C₁˜C₆ alkoxy,
the A ring is phenyl or pyridinyl, and
R₃, R₄ and R₅ are, independently each other, hydrogen, halogen, C₁˜C₆ alkyl, or C₁˜C₆ alkoxy.

In still another embodiment of the present invention,
W is —C(CH₃)₂—,
Q is —CH₂—,
Y is —O—,
X is hydrogen, halogen, C₁˜C₆ alkyl, or C₁˜C₆ alkoxy,
the A ring is phenyl or pyridinyl, and
R₃, R₄ and R₅ are, independently each other, hydrogen, halogen, C₁˜C₆ alkyl, or C₁˜C₆ alkoxy.

In the compound of Formula 1 or pharmaceutically acceptable salt, preferable compounds include a compound, including a pharmaceutically acceptable salt thereof, selected from the group consisting of:
(S)-quinuclidin-3-yl((R)-5-(3-ethylphenyl)-2,2-dimethyl-2,3-dihydro-1H-inden-1-yl)carbamate;
(S)-quinuclidin-3-yl((R)-5-(3-isopropylphenyl)-2,2-dimethyl-2,3-dihydro-1H-inden-1-yl)carbamate;
(S)-quinuclidin-3-yl((R)-5-(4-butylphenyl)-2,2-dimethyl-2,3-dihydro-1H-inden-1-yl)carbamate;
(S)-quinuclidin-3-yl((R)-2,2-dimethyl-5-(4-propylphenyl)-2,3-dihydro-1H-inden-1-yl)carbamate;
(S)-quinuclidin-3-yl((R)-5-(4-isobutylphenyl)-2,2-dimethyl-2,3-dihydro-1H-inden-1-yl)carbamate;
(S)-quinuclidin-3-yl((R)-5-(4-(tert-butyl)phenyl)-2,2-dimethyl-2,3-dihydro-1H-inden-1-yl)carbamate;
(S)-quinuclidin-3-yl((R)-5-(4-ethoxyphenyl)-2,2-dimethyl-2,3-dihydro-1H-inden-1-yl)carbamate;
(S)-quinuclidin-3-yl((R)-5-(4-isobutoxyphenyl)-2,2-dimethyl-2,3-dihydro-1H-inden-1-yl)carbamate;
(S)-quinuclidin-3-yl((R)-5-(3,5-dimethyl-4-propoxyphenyl)-2,2-dimethyl-2,3-dihydro-1H-inden-1-yl)carbamate;
(S)-quinuclidin-3-yl((R)-5-(4-isopropoxy-3,5-dimethylphenyl)-2,2-dimethyl-2,3-dihydro-1H-inden-1-yl)carbamate;
(S)-quinuclidin-3-yl((R)-5-(3-chloro-4-isopropoxyphenyl)-2,2-dimethyl-2,3-dihydro-1H-inden-1-yl)carbamate;
(S)-quinuclidin-3-yl((R)-5-(2-fluoro-4-methoxyphenyl)-2,2-dimethyl-2,3-dihydro-1H-inden-1-yl)carbamate;
(S)-quinuclidin-3-yl((R)-5-(6-(cyclopropylmethoxy)pyridin-3-yl)-2,2-dimethyl-2,3-dihydro-1H-inden-1-yl)carbamate;
(S)-quinuclidin-3-yl((R)-5-(3-chloro-4-methoxyphenyl)-2,2-dimethyl-2,3-dihydro-1H-inden-1-yl)carbamate;
(S)-quinuclidin-3-yl((R)-5-(3-chloro-4-ethoxyphenyl)-2,2-dimethyl-2,3-dihydro-1H-inden-1-yl)carbamate;
(S)-quinuclidin-3-yl((R)-5-(3-chloro-4-propoxyphenyl)-2,2-dimethyl-2,3-dihydro-1H-inden-1-yl)carbamate;
(S)-quinuclidin-3-yl((R)-5-(4-butoxy-3-chlorophenyl)-2,2-dimethyl-2,3-dihydro-1H-inden-1-yl)carbamate;
(S)-quinuclidin-3-yl((R)-5-(3-fluoro-4-methoxyphenyl)-2,2-dimethyl-2,3-dihydro-1H-inden-1-yl)carbamate;
(S)-quinuclidin-3-yl((R)-5-(4-ethoxy-3-fluorophenyl)-2,2-dimethyl-2,3-dihydro-1H-inden-1-yl)carbamate;
(S)-quinuclidin-3-yl((R)-5-(3-fluoro-4-propoxyphenyl)-2,2-dimethyl-2,3-dihydro-1H-inden-1-yl)carbamate;
(S)-quinuclidin-3-yl((R)-5-(3-fluoro-4-isopropoxyphenyl)-2,2-dimethyl-2,3-dihydro-1H-inden-1-yl)carbamate;
(S)-quinuclidin-3-yl((R)-5-(4-ethoxy-3,5-dimethylphenyl)-2,2-dimethyl-2,3-dihydro-1H-inden-1-yl)carbamate;
(S)-quinuclidin-3-yl((R)-5-(3-chloro-4-methoxyphenyl)-2,2-dimethyl-2,3-dihydro-1H-inden-1-yl)carbamate;
(S)-quinuclidin-3-yl((R)-5-(3-chloro-4-ethoxyphenyl)-2,2-dimethyl-2,3-dihydro-1H-inden-1-yl)carbamate;
(S)-quinuclidin-3-yl((R)-5-(3-chloro-4-propoxyphenyl)-2,2-dimethyl-2,3-dihydro-1H-inden-1-yl)carbamate;
(S)-quinuclidin-3-yl((R)-5-(4-butoxy-3-chlorophenyl)-2,2-dimethyl-2,3-dihydro-1H-inden-1-yl)carbamate;
(S)-quinuclidin-3-yl((R)-5-(3-fluoro-4-methoxyphenyl)-2,2-dimethyl-2,3-dihydro-1H-inden-1-yl)carbamate;
(S)-quinuclidin-3-yl((R)-5-(4-ethoxy-3-fluorophenyl)-2,2-dimethyl-2,3-dihydro-1H-inden-1-yl)carbamate;
(S)-quinuclidin-3-yl((R)-5-(3-fluoro-4-propoxyphenyl)-2,2-dimethyl-2,3-dihydro-1H-inden-1-yl)carbamate;
(S)-quinuclidin-3-yl((R)-5-(3-fluoro-4-isopropoxyphenyl)-2,2-dimethyl-2,3-dihydro-1H-inden-1-yl)carbamate;
(S)-quinuclidin-3-yl((R)-5-(4-ethoxy-3,5-dimethylphenyl)-2,2-dimethyl-2,3-dihydro-1H-inden-1-yl)carbamate;
(S)-quinuclidin-3-yl((R)-5-(2-chloro-4-isopropoxyphenyl)-2,2-dimethyl-2,3-dihydro-1H-inden-1-yl)carbamate;
(S)-quinuclidin-3-yl((R)-5-(4-ethyl-3-fluorophenyl)-2,2-dimethyl-2,3-dihydro-1H-inden-1-yl)carbamate;
(S)-quinuclidin-3-yl((R)-5-(4-isopropoxy-3-methylphenyl)-2,2-dimethyl-2,3-dihydro-1H-inden-1-yl)carbamate;
(S)-quinuclidin-3-yl((R)-5-(4-isopropoxy-3-methoxyphenyl)-2,2-dimethyl-2,3-dihydro-1H-inden-1-yl)carbamate;
(S)-quinuclidin-3-yl((R)-5-(4-isopropoxy-2-methylphenyl)-2,2-dimethyl-2,3-dihydro-1H-inden-1-yl)carbamate;
(S)-quinuclidin-3-yl((R)-5-(3,5-difluoro-4-isopropoxyphenyl)-2,2-dimethyl-2,3-dihydro-1H-inden-1-yl)carbamate;
(S)-quinuclidin-3-yl((R)-5-(2-chloro-4-(cyclopropylmethoxy)phenyl)-2,2-dimethyl-2,3-dihydro-1H-inden-1-yl)carbamate;
(S)-quinuclidin-3-yl((R)-5-(3-chloro-4-(cyclopropylmethoxy)phenyl)-2,2-dimethyl-2,3-dihydro-1H-inden-1-yl)carbamate;
(S)-quinuclidin-3-yl((R)-5-(3,5-dichloro-4-isopropoxyphenyl)-2,2-dimethyl-2,3-dihydro-1H-inden-1-yl)carbamate;
(S)-quinuclidin-3-yl((R)-5-(4-(tert-butoxy)phenyl)-2,2-dimethyl-2,3-dihydro-1H-inden-1-yl)carbamate;
(S)-quinuclidin-3-yl((R)-5-(4-(tert-butoxy)-2-chlorophenyl)-2,2-dimethyl-2,3-dihydro-1H-inden-1-yl)carbamate;
(S)-quinuclidin-3-yl((R)-5-(2-chloro-4-methoxyphenyl)-2,2-dimethyl-2,3-dihydro-1H-inden-1-yl)carbamate;
(S)-quinuclidin-3-yl((R)-5-(2-chloro-4-ethoxyphenyl)-2,2-dimethyl-2,3-dihydro-1H-inden-1-yl)carbamate;
(S)-quinuclidin-3-yl((R)-5-(3-chloro-4-methylphenyl)-2,2-dimethyl-2,3-dihydro-1H-inden-1-yl)carbamate;
(S)-quinuclidin-3-yl((R)-5-(4-ethoxy-3-methylphenyl)-2,2-dimethyl-2,3-dihydro-1H-inden-1-yl)carbamate;
(S)-quinuclidin-3-yl((R)-2,2-dimethyl-5-(3-methyl-4-propoxyphenyl)-2,3-dihydro-1H-inden-1-yl)carbamate;
(S)-quinuclidin-3-yl((R)-5-(4-(difluoromethoxy)-3-methylphenyl)-2,2-dimethyl-2,3-dihydro-1H-inden-1-yl)carbamate;
(S)-quinuclidin-3-yl((R)-5-(3-fluoro-5-isopropoxyphenyl)-2,2-dimethyl-2,3-dihydro-1H-inden-1-yl)carbamate;
(S)-quinuclidin-3-yl((R)-5-(3-fluoro-5-isobutoxyphenyl)-2,2-dimethyl-2,3-dihydro-1H-inden-1-yl)carbamate;
(S)-quinuclidin-3-yl((R)-5-(3-fluoro-5-(2,2,2-trifluoroethoxy)phenyl)-2,2-dimethyl-2,3-dihydro-1H-inden-1-yl) carbamate;
(S)-quinuclidin-3-yl((R)-5-(4-chloro-3-isopropoxyphenyl)-2,2-dimethyl-2,3-dihydro-1H-inden-1-yl)carbamate;
(S)-quinuclidin-3-yl((R)-5-(3-chloro-5-isobutoxyphenyl)-2,2-dimethyl-2,3-dihydro-1H-inden-1-yl)carbamate;
(S)-quinuclidin-3-yl((R)-5-(3-chloro-5-isopropoxyphenyl)-2,2-dimethyl-2,3-dihydro-1H-inden-1-yl)carbamate;
(S)-quinuclidin-3-yl((R)-5-(2-chloro-4-(2,2,2-trifluoroethoxy)phenyl)-2,2-dimethyl-2,3-dihydro-1H-inden-1-yl)carbamate;
(S)-quinuclidin-3-yl((R)-5-(4-ethoxyphenyl)-6-fluoro-2,2-dimethyl-2,3-dihydro-1H-inden-1-yl)carbamate;
(S)-quinuclidin-3-yl((R)-6-fluoro-5-(4-isobutoxyphenyl)-2,2-dimethyl-2,3-dihydro-1H-inden-1-yl)carbamate;
(S)-quinuclidin-3-yl((R)-5-(4-ethylphenyl)-6-fluoro-2,2-dimethyl-2,3-dihydro-1H-inden-1-yl)carbamate;
(S)-quinuclidin-3-yl((R)-6-fluoro-5-(4-isopropylphenyl)-2,2-dimethyl-2,3-dihydro-1H-inden-1-yl)carbamate;
(S)-quinuclidin-3-yl((R)-5-(4-butylphenyl)-6-fluoro-2,2-dimethyl-2,3-dihydro-1H-inden-1-yl)carbamate;
(S)-quinuclidin-3-yl((R)-6-fluoro-2,2-dimethyl-5-(4-propylphenyl)-2,3-dihydro-1H-inden-1-yl)carbamate;
(S)-quinuclidin-3-yl((R)-6-fluoro-5-(4-isobutylphenyl)-2,2-dimethyl-2,3-dihydro-1H-inden-1-yl)carbamate;
(S)-quinuclidin-3-yl((R)-6-fluoro-5-(4-methoxy-3,5-dimethylphenyl)-2,2-dimethyl-2,3-dihydro-1H-inden-1-yl)carbamate;
(S)-quinuclidin-3-yl((R)-5-(3,5-dimethyl-4-propoxyphenyl)-6-fluoro-2,2-dimethyl-2,3-dihydro-1H-inden-1-yl)carbamate;
(S)-quinuclidin-3-yl((R)-6-fluoro-5-(4-isopropoxy-3,5-dimethylphenyl)-2,2-dimethyl-2,3-dihydro-1H-inden-1-yl) carbamate;
(S)-quinuclidin-3-yl((R)-5-(3-chloro-4-isopropoxyphenyl)-6-fluoro-2,2-dimethyl-2,3-dihydro-1H-inden-1-yl)carbamate;
(S)-quinuclidin-3-yl((R)-6-fluoro-5-(2-fluoro-4-methoxyphenyl)-2,2-dimethyl-2,3-dihydro-1H-inden-1-yl)carbamate;
(S)-quinuclidin-3-yl((R)-6-fluoro-5-(4-isopropoxyphenyl)-2,2-dimethyl-2,3-dihydro-1H-inden-1-yl)carbamate;
(S)-quinuclidin-3-yl((R)-6-fluoro-2,2-dimethyl-5-(4-propoxyphenyl)-2,3-dihydro-1H-inden-1-yl)carbamate;
(S)-quinuclidin-3-yl((R)-5-(4-butoxyphenyl)-6-fluoro-2,2-dimethyl-2,3-dihydro-1H-inden-1-yl)carbamate;
(S)-quinuclidin-3-yl((R)-5-(3-ethylphenyl)-6-fluoro-2,2-dimethyl-2,3-dihydro-1H-inden-1-yl)carbamate;
(S)-quinuclidin-3-yl((R)-6-fluoro-2,2-dimethyl-5-(3-propylphenyl)-2,3-dihydro-1H-inden-1-yl)carbamate;
(S)-quinuclidin-3-yl((R)-6-fluoro-5-(3-isopropylphenyl)-2,2-dimethyl-2,3-dihydro-1H-inden-1-yl)carbamate;
(S)-quinuclidin-3-yl((R)-6-fluoro-5-(3-isobutylphenyl)-2,2-dimethyl-2,3-dihydro-1H-inden-1-yl)carbamate;
(S)-quinuclidin-3-yl((R)-6-fluoro-5-(3-methoxyphenyl)-2,2-dimethyl-2,3-dihydro-1H-inden-1-yl)carbamate;
(S)-quinuclidin-3-yl((R)-5-(3-ethoxyphenyl)-6-fluoro-2,2-dimethyl-2,3-dihydro-1H-inden-1-yl)carbamate;
(S)-quinuclidin-3-yl((R)-6-fluoro-2,2-dimethyl-5-(3-propoxyphenyl)-2,3-dihydro-1H-inden-1-yl)carbamate;
(S)-quinuclidin-3-yl((R)-6-fluoro-5-(3-isobutoxyphenyl)-2,2-dimethyl-2,3-dihydro-1H-inden-1-yl)carbamate;
(S)-quinuclidin-3-yl((R)-5-(3-butoxyphenyl)-6-fluoro-2,2-dimethyl-2,3-dihydro-1H-inden-1-yl)carbamate;
(S)-quinuclidin-3-yl((R)-5-(4-ethoxy-3,5-dimethylphenyl)-6-fluoro-2,2-dimethyl-2,3-dihydro-1H-inden-1-yl)carbamate;
(S)-quinuclidin-3-yl((R)-6-fluoro-5-(3-isopropoxyphenyl)-2,2-dimethyl-2,3-dihydro-1H-inden-1-yl)carbamate;
(S)-quinuclidin-3-yl((R)-6-fluoro-5-(4-methoxyphenyl)-2,2-dimethyl-2,3-dihydro-1H-inden-1-yl)carbamate;
(S)-quinuclidin-3-yl((R)-6-fluoro-5-(3-fluoro-4-isopropoxyphenyl)-2,2-dimethyl-2,3-dihydro-1H-inden-1-yl)carbamate;
(S)-quinuclidin-3-yl((R)-5-(3-chloro-4-methylphenyl)-6-fluoro-2,2-dimethyl-2,3-dihydro-1H-inden-1-yl)carbamate;
(S)-quinuclidin-3-yl((R)-5-(3-chloro-4-methoxyphenyl)-6-fluoro-2,2-dimethyl-2,3-dihydro-1H-inden-1-yl)carbamate;
(S)-quinuclidin-3-yl((R)-5-(3-chloro-4-ethoxyphenyl)-6-fluoro-2,2-dimethyl-2,3-dihydro-1H-inden-1-yl)carbamate;
(S)-quinuclidin-3-yl((R)-5-(3-chloro-4-propoxyphenyl)-6-fluoro-2,2-dimethyl-2,3-dihydro-1H-inden-1-yl)carbamate;
(S)-quinuclidin-3-yl((R)-5-(4-butoxy-3-chlorophenyl)-6-fluoro-2,2-dimethyl-2,3-dihydro-1H-inden-1-yl)carbamate;
(S)-quinuclidin-3-yl((R)-6-fluoro-5-(3-fluoro-4-methoxyphenyl)-2,2-dimethyl-2,3-dihydro-1H-inden-1-yl)carbamate;
(S)-quinuclidin-3-yl((R)-5-(4-ethoxy-3-fluorophenyl)-6-fluoro-2,2-dimethyl-2,3-dihydro-1H-inden-1-yl)carbamate;
(S)-quinuclidin-3-yl((R)-6-fluoro-5-(3-fluoro-4-propoxyphenyl)-2,2-dimethyl-2,3-dihydro-1H-inden-1-yl)carbamate;
(S)-quinuclidin-3-yl((R)-5-(2-chloro-4-isopropoxyphenyl)-6-fluoro-2,2-dimethyl-2,3-dihydro-1H-inden-1-yl)carbamate;
(S)-quinuclidin-3-yl((R)-5-(4-ethyl-3-fluorophenyl)-6-fluoro-2,2-dimethyl-2,3-dihydro-1H-inden-1-yl)carbamate;
(S)-quinuclidin-3-yl((R)-6-fluoro-5-(4-isopropoxy-3-methylphenyl)-2,2-dimethyl-2,3-dihydro-1H-inden-1-yl)carbamate;
(S)-quinuclidin-3-yl((R)-6-fluoro-5-(4-isopropoxy-3-methoxyphenyl)-2,2-dimethyl-2,3-dihydro-1H-inden-1-yl)carbamate;
(S)-quinuclidin-3-yl((R)-6-fluoro-5-(4-isopropoxy-2-methylphenyl)-2,2-dimethyl-2,3-dihydro-1H-inden-1-yl)carbamate;
(S)-quinuclidin-3-yl((R)-5-(3,5-difluoro-4-isopropoxyphenyl)-6-fluoro-2,2-dimethyl-2,3-dihydro-1H-inden-1-yl)carbamate;
(S)-quinuclidin-3-yl((R)-5-(2-chloro-4-(cyclopropylmethoxy)phenyl)-6-fluoro-2,2-dimethyl-2,3-dihydro-1H-inden-1-yl)carbamate;
(S)-quinuclidin-3-yl((R)-5-(3-chloro-4-(cyclopropylmethoxy)phenyl)-6-fluoro-2,2-dimethyl-2,3-dihydro-1H-inden-1-yl)carbamate;
(S)-quinuclidin-3-yl((R)-5-(3,5-dichloro-4-isopropoxyphenyl)-6-fluoro-2,2-dimethyl-2,3-dihydro-1H-inden-1-yl)carbamate;
(S)-quinuclidin-3-yl((R)-5-(4-(tert-butoxy)phenyl)-6-fluoro-2,2-dimethyl-2,3-dihydro-1H-inden-1-yl)carbamate;
(S)-quinuclidin-3-yl((R)-5-(4-(tert-butoxy)-2-chlorophenyl)-6-fluoro-2,2-dimethyl-2,3-dihydro-1H-inden-1-yl)carbamate;
(S)-quinuclidin-3-yl((R)-5-(2-chloro-4-methoxyphenyl)-6-fluoro-2,2-dimethyl-2,3-dihydro-1H-inden-1-yl)carbamate;
(S)-quinuclidin-3-yl((R)-5-(2-chloro-4-ethoxyphenyl)-6-fluoro-2,2-dimethyl-2,3-dihydro-1H-inden-1-yl) carbamate;
(S)-quinuclidin-3-yl((R)-5-(4-ethoxy-3-methylphenyl)-6-fluoro-2,2-dimethyl-2,3-dihydro-1H-inden-1-yl)carbamate;
(S)-quinuclidin-3-yl((R)-6-fluoro-2,2-dimethyl-5-(3-methyl-4-propoxyphenyl)-2,3-dihydro-1H-inden-1-yl)carbamate;
(S)-quinuclidin-3-yl((R)-5-(4-(difluoromethoxy)-3-methylphenyl)-6-fluoro-2,2-dimethyl-2,3-dihydro-1H-inden-1-yl)carbamate;
(S)-quinuclidin-3-yl((R)-6-fluoro-5-(3-fluoro-5-isopropoxyphenyl)-2,2-dimethyl-2,3-dihydro-1H-inden-1-yl)carbamate;
(S)-quinuclidin-3-yl((R)-6-fluoro-5-(3-fluoro-5-isobutoxyphenyl)-2,2-dimethyl-2,3-dihydro-1H-inden-1-yl)carbamate;
(S)-quinuclidin-3-yl((R)-6-fluoro-5-(3-fluoro-5-(2,2,2-trifluoroethoxy)phenyl)-2,2-dimethyl-2,3-dihydro-1H-inden-1-yl) carbamate;
(S)-quinuclidin-3-yl((R)-5-(4-chloro-3-isopropoxyphenyl)-6-fluoro-2,2-dimethyl-2,3-dihydro-1H-inden-1-yl)carbamate;
(S)-quinuclidin-3-yl((R)-5-(3-chloro-5-isobutoxyphenyl)-6-fluoro-2,2-dimethyl-2,3-dihydro-1H-inden-1-yl)carbamate;
(S)-quinuclidin-3-yl((R)-5-(3-chloro-5-isopropoxyphenyl)-6-fluoro-2,2-dimethyl-2,3-dihydro-1H-inden-1-yl)carbamate;
(S)-quinuclidin-3-yl((R)-5-(2-chloro-4-(2,2,2-trifluoroethoxy)phenyl)-6-fluoro-2,2-dimethyl-2,3-dihydro-1H-inden-1-yl)carbamate;
(S)-quinuclidin-3-yl((R)-5-(4-ethylphenyl)-2,2,6-trimethyl-2,3-dihydro-1H-inden-1-yl)carbamate;
(S)-quinuclidin-3-yl((R)-2,2,6-trimethyl-5-(4-propylphenyl)-2,3-dihydro-1H-inden-1-yl)carbamate;
(S)-quinuclidin-3-yl((R)-5-(4-isopropylphenyl)-2,2,6-trimethyl-2,3-dihydro-1H-inden-1-yl)carbamate;
(S)-quinuclidin-3-yl((R)-5-(4-butylphenyl)-2,2,6-trimethyl-2,3-dihydro-1H-inden-1-yl)carbamate;
(S)-quinuclidin-3-yl((R)-5-(4-isobutylphenyl)-2,2,6-trimethyl-2,3-dihydro-1H-inden-1-yl)carbamate;
(S)-quinuclidin-3-yl((R)-5-(4-ethoxyphenyl)-2,2,6-trimethyl-2,3-dihydro-1H-inden-1-yl)carbamate;
(S)-quinuclidin-3-yl((R)-2,2,6-trimethyl-5-(4-propoxyphenyl)-2,3-dihydro-1H-inden-1-yl)carbamate;
(S)-quinuclidin-3-yl((R)-5-(4-isopropoxyphenyl)-2,2,6-trimethyl-2,3-dihydro-1H-inden-1-yl)carbamate;
(S)-quinuclidin-3-yl((R)-5-(4-butoxyphenyl)-2,2,6-trimethyl-2,3-dihydro-1H-inden-1-yl)carbamate;
(S)-quinuclidin-3-yl((R)-5-(4-isobutoxyphenyl)-2,2,6-trimethyl-2,3-dihydro-1H-inden-1-yl)carbamate;
(S)-quinuclidin-3-yl((R)-5-(3-chloro-4-isopropoxyphenyl)-2,2,6-trimethyl-2,3-dihydro-1H-inden-1-yl)carbamate;
(S)-quinuclidin-3-yl((R)-5-(2-fluoro-4-methoxyphenyl)-2,2,6-trimethyl-2,3-dihydro-1H-inden-1-yl)carbamate;
(S)-quinuclidin-3-yl((R)-6-ethoxy-5-(4-ethylphenyl)-2,2-dimethyl-2,3-dihydro-1H-inden-1-yl)carbamate;
(S)-quinuclidin-3-yl((R)-6-ethoxy-2,2-dimethyl-5-(4-propylphenyl)-2,3-dihydro-1H-inden-1-yl)carbamate;
(S)-quinuclidin-3-yl((R)-6-ethoxy-5-(4-isopropylphenyl)-2,2-dimethyl-2,3-dihydro-1H-inden-1-yl)carbamate;
(S)-quinuclidin-3-yl((R)-5-(4-butylphenyl)-6-ethoxy-2,2-dimethyl-2,3-dihydro-1H-inden-1-yl)carbamate;
(S)-quinuclidin-3-yl((R)-6-ethoxy-5-(4-isobutylphenyl)-2,2-dimethyl-2,3-dihydro-1H-inden-1-yl)carbamate;
(S)-quinuclidin-3-yl((R)-6-ethoxy-5-(4-ethoxyphenyl)-2,2-dimethyl-2,3-dihydro-1H-inden-1-yl)carbamate;
(S)-quinuclidin-3-yl((R)-6-ethoxy-2,2-dimethyl-5-(4-propoxyphenyl)-2,3-dihydro-1H-inden-1-yl)carbamate;
(S)-quinuclidin-3-yl((R)-6-ethoxy-5-(4-isopropoxyphenyl)-2,2-dimethyl-2,3-dihydro-1H-inden-1-yl)carbamate;
(S)-quinuclidin-3-yl((R)-5-(4-butoxyphenyl)-6-ethoxy-2,2-dimethyl-2,3-dihydro-1H-inden-1-yl)carbamate;
(S)-quinuclidin-3-yl((R)-6-ethoxy-5-(4-isobutoxyphenyl)-2,2-dimethyl-2,3-dihydro-1H-inden-1-yl) carbamate;
(S)-quinuclidin-3-yl((R)-5-(3-chloro-4-isopropoxyphenyl)-6-ethoxy-2,2-dimethyl-2,3-dihydro-1H-inden-1-yl)carbamate;
(S)-quinuclidin-3-yl((R)-6-ethoxy-5-(2-fluoro-4-methoxyphenyl)-2,2-dimethyl-2,3-dihydro-1H-inden-1-yl)carbamate;
(S)-quinuclidin-3-yl((R)-6-(4-ethylphenyl)-2,2-dimethyl-1,2,3,4-tetrahydronaphthalen-1-yl)carbamate;
(S)-quinuclidin-3-yl((R)-2,2-dimethyl-6-(4-propylphenyl)-1,2,3,4-tetrahydronaphthalen-1-yl)carbamate;
(S)-quinuclidin-3-yl((R)-6-(4-isopropylphenyl)-2,2-dimethyl-1,2,3,4-tetrahydronaphthalen-1-yl)carbamate;
(S)-quinuclidin-3-yl((R)-6-(4-butylphenyl)-2,2-dimethyl-1,2,3,4-tetrahydronaphthalen-1-yl)carbamate;
(S)-quinuclidin-3-yl((R)-6-(4-ethoxyphenyl)-2,2-dimethyl-1,2,3,4-tetrahydronaphthalen-1-yl)carbamate;
(S)-quinuclidin-3-yl((R)-2,2-dimethyl-6-(4-propoxyphenyl)-1,2,3,4-tetrahydronaphthalen-1-yl)carbamate;
(S)-quinuclidin-3-yl((R)-6-(4-butoxyphenyl)-2,2-dimethyl-1,2,3,4-tetrahydronaphthalen-1-yl)carbamate;
(S)-quinuclidin-3-yl((R)-6-(4-isobutoxyphenyl)-2,2-dimethyl-1,2,3,4-tetrahydronaphthalen-1-yl)carbamate;
(S)-quinuclidin-3-yl((R)-6-(4-methoxy-3,5-dimethylphenyl)-2,2-dimethyl-1,2,3,4-tetrahydronaphthalen-1-yl)carbamate;
(S)-quinuclidin-3-yl((R)-6-(3,5-dimethyl-4-propoxyphenyl)-2,2-dimethyl-1,2,3,4-tetrahydronaphthalen-1-yl)carbamate;
(S)-quinuclidin-3-yl((R)-6-(4-isopropoxy-3,5-dimethylphenyl)-2,2-dimethyl-1,2,3,4-tetrahydronaphthalen-1-yl)carbamate;
(S)-quinuclidin-3-yl((R)-6-(3-chloro-4-isopropoxyphenyl)-2,2-dimethyl-1,2,3,4-tetrahydronaphthalen-1-yl)carbamate;
(S)-quinuclidin-3-yl((R)-6-(2-fluoro-4-methoxyphenyl)-2,2-dimethyl-1,2,3,4-tetrahydronaphthalen-1-yl)carbamate;
(S)-quinuclidin-3-yl((R)-6-(4-butoxy-3,5-dimethylphenyl)-2,2-dimethyl-1,2,3,4-tetrahydronaphthalen-1-yl)carbamate;
(S)-quinuclidin-3-yl((R)-6-(3-methoxyphenyl)-2,2-dimethyl-1,2,3,4-tetrahydronaphthalen-1-yl)carbamate;
(S)-quinuclidin-3-yl((R)-6-(3-ethoxyphenyl)-2,2-dimethyl-1,2,3,4-tetrahydronaphthalen-1-yl)carbamate;
(S)-quinuclidin-3-yl((R)-6-(3-butoxyphenyl)-2,2-dimethyl-1,2,3,4-tetrahydronaphthalen-1-yl)carbamate;
(S)-quinuclidin-3-yl((R)-2,2-dimethyl-6-(3-propylphenyl)-1,2,3,4-tetrahydronaphthalen-1-yl)carbamate;
(S)-quinuclidin-3-yl
((R)-6-(3-isobutylphenyl)-2,2-dimethyl-1,2,3,4-tetrahydronaphthalen-1-yl)carbamate;
(S)-quinuclidin-3-yl((R)-6-(3-ethylphenyl)-2,2-dimethyl-1,2,3,4-tetrahydronaphthalen-1-yl)carbamate;
(S)-quinuclidin-3-yl((R)-2,2-dimethyl-6-(3-propoxyphenyl)-1,2,3,4-tetrahydronaphthalen-1-yl)carbamate;
(S)-quinuclidin-3-yl((R)-6-(4-isobutylphenyl)-2,2-dimethyl-1,2,3,4-tetrahydronaphthalen-1-yl)carbamate;
(S)-quinuclidin-3-yl((R)-6-(3-fluoro-4-isopropoxyphenyl)-2,2-dimethyl-1,2,3,4-tetrahydronaphthalen-1-yl)carbamate;
(S)-quinuclidin-3-yl((R)-6-(3-isopropylphenyl)-2,2-dimethyl-1,2,3,4-tetrahydronaphthalen-1-yl)carbamate;
(S)-quinuclidin-3-yl((R)-6-(3-isobutoxyphenyl)-2,2-dimethyl-1,2,3,4-tetrahydronaphthalen-1-yl)carbamate;
(S)-quinuclidin-3-yl((R)-6-(4-ethoxy-3,5-dimethylphenyl)-2,2-dimethyl-1,2,3,4-tetrahydronaphthalen-1-yl)carbamate;
(S)-quinuclidin-3-yl((R)-6-(3-isopropoxyphenyl)-2,2-dimethyl-1,2,3,4-tetrahydronaphthalen-1-yl)carbamate;
(S)-quinuclidin-3-yl((R)-6-(4-methoxyphenyl)-2,2-dimethyl-1,2,3,4-tetrahydronaphthalen-1-yl)carbamate;
(S)-quinuclidin-3-yl((R)-6-(4-isopropoxyphenyl)-2,2-dimethyl-1,2,3,4-tetrahydronaphthalen-1-yl) carbamate;
(S)-quinuclidin-3-yl((R)-6-(2-chloro-4-isopropoxyphenyl)-2,2-dimethyl-1,2,3,4-tetrahydronaphthalen-1-yl)carbamate;
(S)-quinuclidin-3-yl((R)-6-(4-ethyl-3-fluorophenyl)-2,2-dimethyl-1,2,3,4-tetrahydronaphthalen-1-yl)carbamate;
(S)-quinuclidin-3-yl((R)-6-(4-ethylphenyl)-7-fluoro-2,2-dimethyl-1,2,3,4-tetrahydronaphthalen-1-yl)carbamate;
(S)-quinuclidin-3-yl((R)-7-fluoro-2,2-dimethyl-6-(4-propylphenyl)-1,2,3,4-tetrahydronaphthalen-1-yl)carbamate;
(S)-quinuclidin-3-yl((R)-7-fluoro-6-(4-isopropylphenyl)-2,2-dimethyl-1,2,3,4-tetrahydronaphthalen-1-yl)carbamate;
(S)-quinuclidin-3-yl((R)-6-(4-butylphenyl)-7-fluoro-2,2-dimethyl-1,2,3,4-tetrahydronaphthalen-1-yl)carbamate;
(S)-quinuclidin-3-yl((R)-7-fluoro-6-(4-isobutylphenyl)-2,2-dimethyl-1,2,3,4-tetrahydronaphthalen-1-yl)carbamate;
(S)-quinuclidin-3-yl((R)-7-fluoro-6-(4-methoxyphenyl)-2,2-dimethyl-1,2,3,4-tetrahydronaphthalen-1-yl)carbamate;
(S)-quinuclidin-3-yl((R)-6-(4-ethoxyphenyl)-7-fluoro-2,2-dimethyl-1,2,3,4-tetrahydronaphthalen-1-yl)carbamate;
(S)-quinuclidin-3-yl((R)-7-fluoro-2,2-dimethyl-6-(4-propoxyphenyl)-1,2,3,4-tetrahydronaphthalen-1-yl)carbamate;
(S)-quinuclidin-3-yl((R)-6-(4-butoxyphenyl)-7-fluoro-2,2-dimethyl-1,2,3,4-tetrahydronaphthalen-1-yl)carbamate;
(S)-quinuclidin-3-yl((R)-7-fluoro-6-(4-isobutoxyphenyl)-2,2-dimethyl-1,2,3,4-tetrahydronaphthalen-1-yl)carbamate;
(S)-quinuclidin-3-yl((R)-7-fluoro-6-(4-methoxy-3,5-dimethylphenyl)-2,2-dimethyl-1,2,3,4-tetrahydronaphthalen-1-yl)carbamate;
(S)-quinuclidin-3-yl((R)-6-(3,5-dimethyl-4-propoxyphenyl)-7-fluoro-2,2-dimethyl-1,2,3,4-tetrahydronaphthalen-1-yl)carbamate;
(S)-quinuclidin-3-yl((R)-7-fluoro-6-(4-isopropoxy-3,5-dimethylphenyl)-2,2-dimethyl-1,2,3,4-tetrahydronaphthalen-1-yl)carbamate;
(S)-quinuclidin-3-yl((R)-6-(4-butoxy-3,5-dimethylphenyl)-7-fluoro-2,2-dimethyl-1,2,3,4-tetrahydronaphthalen-1-yl)carbamate;
(S)-quinuclidin-3-yl((R)-7-fluoro-6-(3-methoxyphenyl)-2,2-dimethyl-1,2,3,4-tetrahydronaphthalen-1-yl)carbamate;
(S)-quinuclidin-3-yl((R)-6-(3-ethoxyphenyl)-7-fluoro-2,2-dimethyl-1,2,3,4-tetrahydronaphthalen-1-yl) carbamate;
(S)-quinuclidin-3-yl((R)-6-(3-butoxyphenyl)-7-fluoro-2,2-dimethyl-1,2,3,4-tetrahydronaphthalen-1-yl)carbamate;
(S)-quinuclidin-3-yl((R)-6-(3-ethylphenyl)-7-fluoro-2,2-dimethyl-1,2,3,4-tetrahydronaphthalen-1-yl)carbamate;
(S)-quinuclidin-3-yl((R)-7-fluoro-2,2-dimethyl-6-(3-propylphenyl)-1,2,3,4-tetrahydronaphthalen-1-yl)carbamate;
(S)-quinuclidin-3-yl((R)-7-fluoro-6-(3-isobutylphenyl)-2,2-dimethyl-1,2,3,4-tetrahydronaphthalen-1-yl)carbamate;
(S)-quinuclidin-3-yl((R)-7-fluoro-6-(3-isopropylphenyl)-2,2-dimethyl-1,2,3,4-tetrahydronaphthalen-1-yl)carbamate;
(S)-quinuclidin-3-yl((R)-7-fluoro-6-(3-isobutoxyphenyl)-2,2-dimethyl-1,2,3,4-tetrahydronaphthalen-1-yl)carbamate;
(S)-quinuclidin-3-yl((R)-7-fluoro-2,2-dimethyl-6-(3-propoxyphenyl)-1,2,3,4-tetrahydronaphthalen-1-yl)carbamate;
(S)-quinuclidin-3-yl((R)-6-(3-chloro-4-isopropoxyphenyl)-7-fluoro-2,2-dimethyl-1,2,3,4-tetrahydronaphthalen-1-yl)carbamate;
(S)-quinuclidin-3-yl((R)-7-fluoro-6-(2-fluoro-4-methoxyphenyl)-2,2-dimethyl-1,2,3,4-tetrahydronaphthalen-1-yl)carbamate;
(S)-quinuclidin-3-yl((R)-6-(4-ethoxy-3,5-dimethylphenyl)-7-fluoro-2,2-dimethyl-1,2,3,4-tetrahydronaphthalen-1-yl)carbamate;
(S)-quinuclidin-3-yl((R)-7-fluoro-6-(3-isopropoxyphenyl)-2,2-dimethyl-1,2,3,4-tetrahydronaphthalen-1-yl)carbamate;
(S)-quinuclidin-3-yl((R)-7-fluoro-6-(4-isopropoxyphenyl)-2,2-dimethyl-1,2,3,4-tetrahydronaphthalen-1-yl)carbamate;
(S)-quinuclidin-3-yl((R)-7-fluoro-6-(3-fluoro-4-isopropoxyphenyl)-2,2-dimethyl-1,2,3,4-tetrahydronaphthalen-1-yl)carbamate; and
(S)-quinuclidin-3-yl((R)-6-(2-chloro-4-isopropoxyphenyl)-7-fluoro-2,2-dimethyl-1,2,3,4-tetrahydronaphthalen-1-yl)carbamate.
In the compound of Formula 1 or pharmaceutically acceptable salt, more preferable compounds include a compound, including a pharmaceutically acceptable salt thereof,
selected from the group consisting of:
(S)-quinuclidin-3-yl((R)-5-(3,5-dimethyl-4-propoxyphenyl)-2,2-dimethyl-2,3-dihydro-1H-inden-1-yl)carbamate;
((R)-5-(4-isopropoxy-3,5-dimethylphenyl)-2,2-dimethyl-2,3-(S)-quinuclidin-3-yl dihydro-1H-inden-1-yl)carbamate;
(S)-quinuclidin-3-yl((R)-5-(3-chloro-4-isopropoxyphenyl)-2,2-dimethyl-2,3-dihydro-1H-inden-1-yl)carbamate;
(S)-quinuclidin-3-yl((R)-5-(3-chloro-4-methoxyphenyl)-2,2-dimethyl-2,3-dihydro-1H-inden-1-yl)carbamate;
(S)-quinuclidin-3-yl((R)-5-(3-chloro-4-ethoxyphenyl)-2,2-dimethyl-2,3-dihydro-1H-inden-1-yl)carbamate;
(S)-quinuclidin-3-yl((R)-5-(3-chloro-4-propoxyphenyl)-2,2-dimethyl-2,3-dihydro-1H-inden-1-yl)carbamate;
(S)-quinuclidin-3-yl((R)-5-(3-fluoro-4-methoxyphenyl)-2,2-dimethyl-2,3-dihydro-1H-inden-1-yl)carbamate;
(S)-quinuclidin-3-yl((R)-5-(3-fluoro-4-propoxyphenyl)-2,2-dimethyl-2,3-dihydro-1H-inden-1-yl)carbamate;
(S)-quinuclidin-3-yl((R)-5-(3-fluoro-4-isopropoxyphenyl)-2,2-dimethyl-2,3-dihydro-1H-inden-1-yl)carbamate;
(S)-quinuclidin-3-yl((R)-5-(4-ethoxy-3,5-dimethylphenyl)-2,2-dimethyl-2,3-dihydro-1H-inden-1-yl)carbamate;
(S)-quinuclidin-3-yl((R)-5-(2-chloro-4-isopropoxyphenyl)-2,2-dimethyl-2,3-dihydro-1H-inden-1-yl)carbamate;
(S)-quinuclidin-3-yl((R)-5-(4-isopropoxy-3-methylphenyl)-2,2-dimethyl-2,3-dihydro-1H-inden-1-yl)carbamate;
(S)-quinuclidin-3-yl((R)-5-(4-isopropoxy-3-methoxyphenyl)-2,2-dimethyl-2,3-dihydro-1H-inden-1-yl)carbamate;
(S)-quinuclidin-3-yl((R)-5-(4-isopropoxy-2-methylphenyl)-2,2-dimethyl-2,3-dihydro-1H-inden-1-yl)carbamate;
(S)-quinuclidin-3-yl((R)-5-(2-chloro-4-(cyclopropylmethoxy)phenyl)-2,2-dimethyl-2,3-dihydro-1H-inden-1-yl)carbamate;
(S)-quinuclidin-3-yl((R)-5-(3-chloro-4-(cyclopropylmethoxy)phenyl)-2,2-dimethyl-2,3-dihydro-1H-inden-1-yl)carbamate;
(S)-quinuclidin-3-yl((R)-5-(2-chloro-4-methoxyphenyl)-2,2-dimethyl-2,3-dihydro-1H-inden-1-yl)carbamate;
(S)-quinuclidin-3-yl((R)-5-(2-chloro-4-ethoxyphenyl)-2,2-dimethyl-2,3-dihydro-1H-inden-1-yl)carbamate;
(S)-quinuclidin-3-yl((R)-5-(3-chloro-4-methylphenyl)-2,2-dimethyl-2,3-dihydro-1H-inden-1-yl)carbamate;
(S)-quinuclidin-3-yl((R)-6-fluoro-5-(4-isobutoxyphenyl)-2,2-dimethyl-2,3-dihydro-1H-inden-1-yl)carbamate;
(S)-quinuclidin-3-yl((R)-6-fluoro-5-(4-isopropylphenyl)-2,2-dimethyl-2,3-dihydro-1H-inden-1-yl)carbamate;
(S)-quinuclidin-3-yl((R)-6-fluoro-2,2-dimethyl-5-(4-propylphenyl)-2,3-dihydro-1H-inden-1-yl)carbamate;
(S)-quinuclidin-3-yl((R)-6-fluoro-5-(4-isobutylphenyl)-2,2-dimethyl-2,3-dihydro-1H-inden-1-yl)carbamate;
(S)-quinuclidin-3-yl((R)-6-fluoro-5-(4-methoxy-3,5-dimethylphenyl)-2,2-dimethyl-2,3-dihydro-1H-inden-1-yl)carbamate;
(S)-quinuclidin-3-yl((R)-5-(3,5-dimethyl-4-propoxyphenyl)-6-fluoro-2,2-dimethyl-2,3-dihydro-1H-inden-1-yl)carbamate;
(S)-quinuclidin-3-yl((R)-6-fluoro-5-(4-isopropoxy-3,5-dimethylphenyl)-2,2-dimethyl-2,3-dihydro-1H-inden-1-yl)carbamate;
(S)-quinuclidin-3-yl((R)-5-(3-chloro-4-isopropoxyphenyl)-6-fluoro-2,2-dimethyl-2,3-dihydro-1H-inden-1-yl)carbamate;
(S)-quinuclidin-3-yl((R)-6-fluoro-5-(2-fluoro-4-methoxyphenyl)-2,2-dimethyl-2,3-dihydro-1H-inden-1-yl)carbamate;
(S)-quinuclidin-3-yl((R)-6-fluoro-5-(4-isopropoxyphenyl)-2,2-dimethyl-2,3-dihydro-1H-inden-1-yl)carbamate;
(S)-quinuclidin-3-yl((R)-6-fluoro-2,2-dimethyl-5-(4-propoxyphenyl)-2,3-dihydro-1H-inden-1-yl)carbamate;
(S)-quinuclidin-3-yl((R)-6-fluoro-5-(4-methoxyphenyl)-2,2-dimethyl-2,3-dihydro-1H-inden-1-yl)carbamate;
(S)-quinuclidin-3-yl((R)-6-fluoro-5-(3-fluoro-4-isopropoxyphenyl)-2,2-dimethyl-2,3-dihydro-1H-inden-1-yl) carbamate;
(S)-quinuclidin-3-yl((R)-5-(3-chloro-4-methylphenyl)-6-fluoro-2,2-dimethyl-2,3-dihydro-1H-inden-1-yl)carbamate;
(S)-quinuclidin-3-yl((R)-5-(3-chloro-4-ethoxyphenyl)-6-fluoro-2,2-dimethyl-2,3-dihydro-1H-inden-1-yl)carbamate;
(S)-quinuclidin-3-yl((R)-5-(3-chloro-4-propoxyphenyl)-6-fluoro-2,2-dimethyl-2,3-dihydro-1H-inden-1-yl)carbamate;
(S)-quinuclidin-3-yl((R)-6-fluoro-5-(3-fluoro-4-methoxyphenyl)-2,2-dimethyl-2,3-dihydro-1H-inden-1-yl)carbamate;
(S)-quinuclidin-3-yl((R)-5-(4-ethoxy-3-fluorophenyl)-6-fluoro-2,2-dimethyl-2,3-dihydro-1H-inden-1-yl)carbamate;
(S)-quinuclidin-3-yl((R)-5-(2-chloro-4-isopropoxyphenyl)-6-fluoro-2,2-dimethyl-2,3-dihydro-1H-inden-1-yl) carbamate;
(S)-quinuclidin-3-yl((R)-6-fluoro-5-(4-isopropoxy-2-methylphenyl)-2,2-dimethyl-2,3-dihydro-1H-inden-1-yl)carbamate;
(S)-quinuclidin-3-yl((R)-5-(2-chloro-4-methoxyphenyl)-6-fluoro-2,2-dimethyl-2,3-dihydro-1H-inden-1-yl)carbamate;
(S)-quinuclidin-3-yl((R)-5-(2-chloro-4-ethoxyphenyl)-6-fluoro-2,2-dimethyl-2,3-dihydro-1H-inden-1-yl) carbamate;
(S)-quinuclidin-3-yl((R)-6-(3,5-dimethyl-4-propoxyphenyl)-2,2-dimethyl-1,2,3,4-tetrahydronaphthalen-1-yl)carbamate;
(S)-quinuclidin-3-yl((R)-6-(3-chloro-4-isopropoxyphenyl)-2,2-dimethyl-1,2,3,4-tetrahydronaphthalen-1-yl)carbamate;
(S)-quinuclidin-3-yl((R)-6-(2-fluoro-4-methoxyphenyl)-2,2-dimethyl-1,2,3,4-tetrahydronaphthalen-1-yl)carbamate;
(S)-quinuclidin-3-yl((R)-6-(3-ethylphenyl)-2,2-dimethyl-1,2,3,4-tetrahydronaphthalen-1-yl)carbamate;
(S)-quinuclidin-3-yl((R)-6-(3-fluoro-4-isopropoxyphenyl)-2,2-dimethyl-1,2,3,4-tetrahydronaphthalen-1-yl)carbamate;
(S)-quinuclidin-3-yl((R)-6-(3-isopropylphenyl)-2,2-dimethyl-1,2,3,4-tetrahydronaphthalen-1-yl)carbamate;
(S)-quinuclidin-3-yl((R)-6-(3-isopropoxyphenyl)-2,2-dimethyl-1,2,3,4-tetrahydronaphthalen-1-yl)carbamate;
(S)-quinuclidin-3-yl((R)-6-(2-chloro-4-isopropoxyphenyl)-2,2-dimethyl-1,2,3,4-tetrahydronaphthalen-1-yl)carbamate;
(S)-quinuclidin-3-yl((R)-7-fluoro-2,2-dimethyl-6-(4-propylphenyl)-1,2,3,4-tetrahydronaphthalen-1-yl)carbamate;
(S)-quinuclidin-3-yl((R)-6-(4-ethoxyphenyl)-7-fluoro-2,2-dimethyl-1,2,3,4-tetrahydronaphthalen-1-yl)carbamate;
(S)-quinuclidin-3-yl((R)-7-fluoro-2,2-dimethyl-6-(4-propoxyphenyl)-1,2,3,4-tetrahydronaphthalen-1-yl)carbamate;
(S)-quinuclidin-3-yl((R)-7-fluoro-6-(4-methoxy-3,5-dimethylphenyl)-2,2-dimethyl-1,2,3,4-tetrahydronaphthalen-1-yl)carbamate;
(S)-quinuclidin-3-yl((R)-6-(3,5-dimethyl-4-propoxyphenyl)-7-fluoro-2,2-dimethyl-1,2,3,4-tetrahydronaphthalen-1-yl)carbamate;
(S)-quinuclidin-3-yl((R)-6-(3-chloro-4-isopropoxyphenyl)-7-fluoro-2,2-dimethyl-1,2,3,4-tetrahydronaphthalen-1-yl)carbamate;
(S)-quinuclidin-3-yl((R)-7-fluoro-6-(2-fluoro-4-methoxyphenyl)-2,2-dimethyl-1,2,3,4-tetrahydronaphthalen-1-yl)carbamate;
(S)-quinuclidin-3-yl((R)-7-fluoro-6-(4-isopropoxyphenyl)-2,2-dimethyl-1,2,3,4-tetrahydronaphthalen-1-yl)carbamate;
(S)-quinuclidin-3-yl((R)-7-fluoro-6-(3-fluoro-4-isopropoxyphenyl)-2,2-dimethyl-1,2,3,4-tetrahydronaphthalen-1-yl)carbamate; and
(S)-quinuclidin-3-yl((R)-6-(2-chloro-4-isopropoxyphenyl)-7-fluoro-2,2-dimethyl-1,2,3,4-tetrahydronaphthalen-1-yl)carbamate.
In the compound of Formula 1 or pharmaceutically acceptable salt, still more preferable compounds include a compound, including a pharmaceutically acceptable salt thereof,
selected from the group consisting of:
(S)-quinuclidin-3-yl((R)-5-(3-chloro-4-isopropoxyphenyl)-2,2-dimethyl-2,3-dihydro-1H-inden-1-yl)carbamate;
(S)-quinuclidin-3-yl((R)-5-(2-fluoro-4-methoxyphenyl)-2,2-dimethyl-2,3-dihydro-1H-inden-1-yl)carbamate;
(S)-quinuclidin-3-yl((R)-5-(3-chloro-4-propoxyphenyl)-2,2-dimethyl-2,3-dihydro-1H-inden-1-yl)carbamate;
(S)-quinuclidin-3-yl((R)-5-(3-fluoro-4-isopropoxyphenyl)-2,2-dimethyl-2,3-dihydro-1H-inden-1-yl)carbamate;
(S)-quinuclidin-3-yl((R)-5-(2-chloro-4-isopropoxyphenyl)-2,2-dimethyl-2,3-dihydro-1H-inden-1-yl)carbamate;
(S)-quinuclidin-3-yl((R)-5-(4-isopropoxy-3-methylphenyl)-2,2-dimethyl-2,3-dihydro-1H-inden-1-yl)carbamate;
(S)-quinuclidin-3-yl((R)-5-(3-chloro-4-methylphenyl)-2,2-dimethyl-2,3-dihydro-1H-inden-1-yl)carbamate;
(S)-quinuclidin-3-yl((R)-5-(3-chloro-4-isopropoxyphenyl)-6-fluoro-2,2-dimethyl-2,3-dihydro-1H-inden-1-yl)carbamate;
(S)-quinuclidin-3-yl((R)-6-fluoro-5-(3-fluoro-4-isopropoxyphenyl)-2,2-dimethyl-2,3-dihydro-1H-inden-1-yl)carbamate;
(S)-quinuclidin-3-yl((R)-6-fluoro-5-(4-isopropoxy-2-methylphenyl)-2,2-dimethyl-2,3-dihydro-1H-inden-1-yl)carbamate;
(S)-quinuclidin-3-yl((R)-6-(3-fluoro-4-isopropoxyphenyl)-2,2-dimethyl-1,2,3,4-tetrahydronaphthalen-1-yl)carbamate;
(S)-quinuclidin-3-yl((R)-6-(2-chloro-4-isopropoxyphenyl)-2,2-dimethyl-1,2,3,4-tetrahydronaphthalen-1-yl)carbamate;
(S)-quinuclidin-3-yl((R)-7-fluoro-2,2-dimethyl-6-(4-propoxyphenyl)-1,2,3,4-tetrahydronaphthalen-1-yl)carbamate; and
(S)-quinuclidin-3-yl((R)-6-(3-chloro-4-isopropoxyphenyl)-7-fluoro-2,2-dimethyl-1,2,3,4-tetrahydronaphthalen-1-yl)carbamate.

The compound of Formula 1 of the present invention may be in a pharmaceutically acceptable salt form. The salt may be a conventional acid addition salt form, which includes e.g., salts derived from an inorganic acid such as hydrochloric acid, sulfuric acid, nitric acid, phosphoric acid, perchloric acid, or hydrobromic acid; and salts derived from an organic acid such as formic acid, acetic acid, propionic acid, oxalic acid, succinic acid, benzoic acid, citric acid, maleic acid, malonic acid, malic acid, tartaric acid, gluconic acid, lactic acid, gentisic acid, fumaric acid, lactobionic acid, salicylic acid, phthalic acid, embonic acid, aspartic acid, glutamic acid, or acetylsalicylic acid. And, the salt also includes, e.g., salts derived from an amino acid such as glycine, alanine, valine, isoleucine, serine, cysteine, cystine, aspartic acid, glutamine, lysine, arginine, tyrosine, or proline. In addition, the salt includes, e.g., salts derived from a sulfonic acid such as methanesulfonic acid, ethanesulfonic acid, benzenesulfonic acid, or toluenesulfonic acid.

The compound of Formula 1 or pharmaceutically acceptable salt thereof according the present invention may be prepared according to various methods.

For example, the compound of Formula 1 or pharmaceutically acceptable salt thereof according the present invention may be prepared by a process which comprises condensing a compound of Formula 2 and 2-methylpropane-2-sulfinamide to obtain a compound of Formula 3; reducing the compound of Formula 3 to obtain a compound of Formula 4; reacting the compound of Formula 4 with an acid to obtain a compound of Formula 5; reacting the compound of Formula 5 with quinuclidinol and bis(4-nitrophenyl) carbonate to obtain a compound of Formula 6; reacting the compound of Formula 6 with a (phenyl or pyridinyl)-boronic acid substituted with R₃, R₄, or R₅ to obtain a compound of Formula 1; and optionally converting the compound of Formula 1 to a pharmaceutically acceptable salt thereof, as shown in the following Reaction Scheme 1.

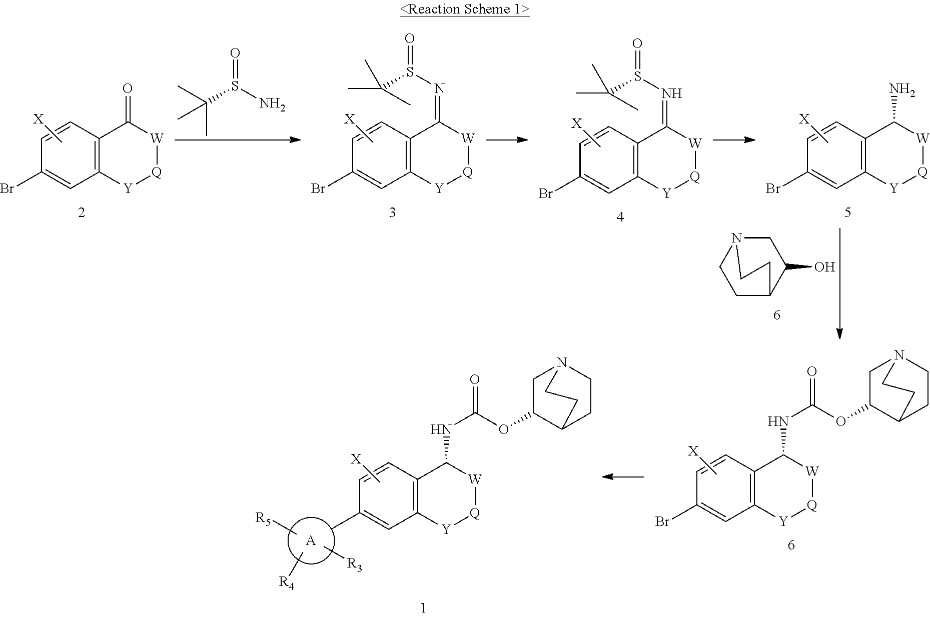

In the Reaction Scheme 1, W, Q, X, Y, A, R₃, R₄, and R₅ are the same as defined in the above.

The compound of Formula 2, which is commercially available or a known compound, may be synthesized according to literatures. The condensation of the compound of Formula 2 and 2-methylpropane-2-sulfinamide may be carried out in the presence of a Lewis acid catalyst such as titanium isopropoxide or titanium ethoxide. The condensation may be carried out in an organic solvent such as ethyl acetate, dichloromethane, tetrahydrofuran, or toluene at 60° C. to 120° C.

The reduction of the compound of Formula 3 may be carried out with a reducing agent such as sodium borohydride. Through said reduction, the (S)-sulfinamide derivative of Formula 4 can be obtained in a diastereoisomeric ratio of 90:10 to 97:3. The reduction may be carried out, for example, in a solvent such as tetrahydrofuran at −50° C. to 0° C.

The reaction between the compound of Formula 4 and an acid may give the amine derivative of Formula 5 in which the sulfinyl group has been removed. The reaction may be carried out in a solvent such as ethyl acetate or acetonitrile at 0° C. to room temperature. If necessary, the amine derivative of Formula 5 may be obtained in the form of a hydrochloride salt by adding hydrochloric acid thereto.

The reaction between the compound of Formula 5 and quinuclidinol/bis(4-nitrophenyl) carbonate may be carried out in a polar solvent such as N,N-dimethylformamide, tetrahydrofuran, or acetonitrile. The reaction may be carried out in the presence of a base such as N,N-diisopropylethylamine or triethylamine at 0° C. to 25° C.

The reaction between the compound of Formula 6 and a (phenyl or pyridinyl)-boronic acid substituted with R₃, R₄, or R₅ may be carried out according to the Suzuki reaction. Said Suzuki reaction may be carried out using a palladium catalyst, such as tetrakis(triphenylphosphine)palladium(0) (Pd(PPh₃)₄), palladium(II) acetate (Pd(OAc)₂), [1,1′-bis(diphenylphosphino)ferrocene]dichloropalladium(II) (Pd(dppf)Cl₂), etc. In addition, said Suzuki reaction may be carried out in the presence of an inorganic base such as cesium carbonate (CS₂CO₃), sodium carbonate (Na₂CO₃), potassium carbonate (K₂CO₃), potassium phosphate (K₃PO₄), etc. Said Suzuki reaction may be carried out in a non-polar organic solvent such as toluene or a polar organic solvent such as 1,4-dioxane, tetrahydrofuran, acetonitrile, 1,2-dimethoxyethane, or N, N-dimethylformamide, at 50° C. to 150° C., preferably 80° C. to 120° C. Other reaction conditions including a reaction time may be determined according to known methods for the Suzuki reaction.

The conversion of the compound of Formula 1 to a pharmaceutically acceptable salt thereof may be carried out according to conventional methods. For example, a pharmaceutically acceptable salt of the compound of Formula 1 may be prepared by adding an inorganic acid to the compound of Formula 1 in an organic solvent such as diisopropyl ether or ethyl acetate; or by dissolving the compound of Formula 1 in a water-miscible solvent such as methanol, ethanol, acetone or 1,4-dioxane and then adding a free acid thereto for the crystallization thereof.

The compound of Formula 1 or pharmaceutically acceptable salt thereof according to the present invention has an excellent inhibitory activity against glucosylceramide synthase (GCS), and therefore can be usefully applied for preventing or treating various diseases associated with GCS.

Therefore, the present invention includes, within its scope, a pharmaceutical composition for inhibiting glucosylceramide synthase (GCS), comprising a therapeutically effective amount of the compound of Formula 1 or pharmaceutically acceptable salt thereof as an active ingredient. In an embodiment, the present invention provides a pharmaceutical composition for preventing or treating the diseases associated with GCS, such as Gaucher disease, Fabry disease, Tay-Sachs disease, Parkinson's disease, etc., comprising a therapeutically effective amount of the compound of Formula 1 or pharmaceutically acceptable salt thereof as an active ingredient.

The pharmaceutical composition of the present invention may comprise a conventional pharmaceutically acceptable carrier, such as diluents, disintegrants, sweeteners, lubricants, or flavoring agents. The pharmaceutical composition may be formulated to an oral dosage form such as tablets, capsules, powders, granules, suspensions, emulsions, or syrups; or a parenteral dosage form such as solutions for external use, suspensions for external use, emulsions for external use, gels (e.g., ointment), inhalations, nebulizations, injections. The dosage form may be various forms, e.g., dosage forms for single administration or for multiple administrations.

The pharmaceutical composition of the present invention may comprise, for example, a diluent (e.g., lactose, corn starch, etc); a lubricant (e.g., magnesium stearate); an emulsifying agent; a suspending agent; a stabilizer; and/or an isotonic agent. If necessary, the composition further comprises sweeteners and/or flavoring agents.

The composition of the present invention may be administered orally or parenterally, including inhalant, intravenous, intraperitoneal, subcutaneous, rectal and topical routes of administration. Therefore, the composition of the present invention may be formulated into various forms such as tablets, capsules, aqueous solutions or suspensions. In the case of tablets for oral administration, carriers such as lactose, corn starch, and lubricating agents, e.g. magnesium stearate, are conventionally used. In the case of capsules for oral administration, lactose and/or dried corn starch can be used as a diluent. When an aqueous suspension is required for oral administration, the active ingredient may be combined with emulsifying and/or suspending agents. If desired, certain sweetening and/or flavoring agents may be used. For intramuscular, intraperitoneal, subcutaneous and intravenous administration, sterile solutions of the active ingredient are usually prepared, and the pH of the solutions should be suitably adjusted and buffered. For intravenous administration, the total concentration of solutes should be controlled in order to render the preparation isotonic. The composition of the present invention may be in the form of an aqueous solution containing pharmaceutically acceptable carriers, e.g., saline having a pH level of 7.4. The solutions may be introduced into a patient's intramuscular blood-stream by local bolus injection.

The compound of Formula 1 or pharmaceutically acceptable salt thereof may be administered in a therapeutically effective amount ranging from about 0.0001 mg/kg to about 100 mg/kg per day to a subject patient. Of course, the dosage may be changed according to the patient's age, weight, susceptibility, symptom, or activity of the compound.

The present invention includes, within its scope, a method for inhibiting glucosylceramide synthase (GCS) in a mammal, comprising administering a therapeutically effective amount of the compound of Formula 1 or pharmaceutically acceptable salt thereof to the mammal in need thereof. In an embodiment, the present invention provides a method for treating the diseases associated with GCS, such as Gaucher disease, Fabry disease, Tay-Sachs disease, Parkinson's disease, etc., comprising administering a therapeutically effective amount of the compound of Formula 1 or pharmaceutically acceptable salt thereof to the mammal in need thereof.

The present invention also provides a use of the compound of Formula 1 or pharmaceutically acceptable salt thereof for the manufacture of a medicament for inhibiting glucosylceramide synthase (GCS) in a mammal. In an embodiment, the present invention provides a use of the compound of Formula 1 or pharmaceutically acceptable salt thereof for the manufacture of a medicament for preventing or treating the diseases associated with GCS, such as Gaucher disease, Fabry disease, Tay-Sachs disease, Parkinson's disease, etc.

The following examples and experimental examples are provided for illustration purposes only, and are not intended to limit the scope of the invention.

In the following examples, brine refers to a saturated aqueous sodium chloride solution. Unless otherwise indicated, all temperatures are in degrees Celsius (° C.). All reactions were carried out at room temperature unless otherwise indicated.

The analyses of the compounds prepared in the following Preparations and Examples were carried out as follows: Nuclear magnetic resonance (NMR) spectrum analysis was carried out using Bruker 400 MHz spectrometer and chemical shifts thereof were analyzed in ppm. Column chromatography was carried out on silica gel (Merck, 70-230 mesh). Each starting material is a known compound which was synthesized according to literatures or purchased commercially. All reactions and chromatographic fractions were analyzed by thin layer chromatography (TLC) on a 250 nm silica gel plate and visualized with ultraviolet or iodine (I₂) staining.

### Preparation 1. (S)-quinuclidin-3-yl((R)-5-bromo-2,2-dimethyl-2,3-dihydro-1H-inden-1-yl)carbamate

### Step 1. (S,Z)-N-(5-bromo-2,2-dimethyl-2,3-dihydro-1H-inden-1-ylidene)-2-methylpropane-2-sulfinamide

To a solution of 5-bromo-2,2-dimethyl-2,3-dihydro-1H-inden-1-one (100 g, 0.42 mol) in toluene (1 L), were added (S)-2-methylpropane-2-sulfinamide (101.4 g, 0.84 mol) and titanium(IV) ethoxide (190.8 g, 0.84 mol). The reaction mixture was stirred at 120° C. overnight and then cooled to room temperature. The reaction mixture was poured into ice water (1.5 L) and filtered under reduced pressure. The filtrate was extracted with ethyl acetate. The resulting organic phase was washed with brine, dried over magnesium sulfate, filtered, and then concentrated under reduced pressure. The resulting residue was purified by silica gel column chromatography (n-hexane/ethyl acetate=10/1, v/v) to give the titled compound as yellow oil. (70 g, Yield: 49%)

¹H-NMR (400 MHZ, CDCl₃) δ 8.30 (br, 1H), 7.52 (s, 1H), 7.47 (d, 1H), 2.97 (m, 2H), 1.32 (s, 15H)

### Step 2. (S)—N—((R)-5-bromo-2,2-dimethyl-2,3-dihydro-1H-inden-1-yl)-2-methylpropane-2-sulfinamide

(S,Z)-N-(5-bromo-2,2-dimethyl-2,3-dihydro-1H-inden-1-ylidene)-2-methylpropane-2-sulfinamide (40 g, 0.12 mol) prepared in Step 1 was dissolved in tetrahydrofuran (400 mL) and then sodium borohydride (13.3 g, 0.35 mol) was slowly added thereto at −40° C. The reaction mixture was slowly warmed to room temperature and then stirred at room temperature overnight. The reaction mixture was poured into water (500 mL) and extracted with ethyl acetate. The resulting organic phase was dried over magnesium sulfate, filtered, and then concentrated under reduced pressure. The resulting residue was purified by silica gel column chromatography (n-hexane/ethyl acetate=5/1, v/v) to give the titled compound as a white solid. (24 g, Yield: 60%)

¹H-NMR (400 MHZ, CDCl₃) δ 7.32 (m, 2H), 7.12 (d, 1H), 4.40 (d, 1H), 3.49 (d, 1H), 2.79˜2.67(q, 2H), 1.68 (s, 3H), 1.31 (s, 9H), 0.96 (s, 3H)

### Step 3. (R)-5-bromo-2,2-dimethyl-2,3-dihydro-1H-inden-1-amine

(S)—N—((R)-5-bromo-2,2-dimethyl-2,3-dihydro-1H-inden-1-yl)-2-methylpropane-2-sulfinamide (35 g, 0.10 mol) prepared in Step 2 was dissolved in ethyl acetate (350 mL) and conc. hydrochloric acid (35 mL) was added thereto at 0° C. The reaction mixture was stirred at room temperature for 1 hour. The reaction mixture was poured into water (300 mL) and then the aqueous layer was extracted. The aqueous phase was adjusted pH to 8-9 with Na₂CO₃ and extracted with ethyl acetate (3×300 mL). The resulting organic layer was dried over magnesium sulfate, filtered, and then concentrated under reduced pressure to give the titled compound as yellow oil. (20 g, Yield: 82%)

### Step 4. (S)-quinuclidin-3-yl((R)-5-bromo-2,2-dimethyl-2,3-dihydro-1H-inden-1-yl)carbamate

A solution of (S)-(+)-3-quinuclidinol (12.71 g, 99.94 mmol) and bis(4-nitrophenyl) carbonate (30.4 g, 99.94 mmol) in N,N-dimethylformamide (500 ml) was stirred at room temperature for 8 hours. (R)-5-bromo-2,2-dimethyl-2,3-dihydro-1H-inden-1-amine (20 g, 83.29 mmol) prepared in Step 3 and diisopropylethylamine (17.41 mL, 99.94 mmol) were added to the solution, which was then stirred at room temperature overnight. The reaction mixture was diluted with water (500 mL) and then acidified to pH 1-2 with a 1N hydrochloric acid solution. The organic layer was separated using diisopropyl ether (300 mL×2). The resulting aqueous layer was basified to about pH 12 with ammonia water (1.5 L) and extracted with ethyl acetate. The extract was washed with water, dried over magnesium sulfate, filtered, and concentrated under reduced pressure. The resulting residue was dissolved in isopropyl acetate and then n-heptane was added thereto. The mixture was stirred at room temperature overnight. The resulting solid was filtered, washed with n-heptane, and dried to give the titled compound as a white solid. (28 g, Yield: 85%)

¹H-NMR (400 MHZ, CDCl₃) δ 7.32 (m, 2H), 7.09 (d, 1H), 4.89 (m, 2H), 4.76 (m, 1H), 3.26 (m, 1H), 2.90˜2.66 (m, 7H), 2.08 (m, 1H), 1.83 (m, 1H), 1.69 (m, 1H), 1.58 (m, 1H), 1.42 (m, 1H), 1.28 (s, 3H), 0.93 (s, 3H)

### Preparation 2. (S)-quinuclidin-3-yl((R)-5-bromo-6-fluoro-2,2-dimethyl-2,3-dihydro-1H-inden-1-yl)carbamate

### Step 1.5-bromo-6-fluoro-2,2-dimethyl-2,3-dihydro-1H-inden-1-one

To a solution of 5-Bromo-6-fluoroindan-1-one (25 g, 109.15 mmol) in tetrahydrofuran (300 mL) was added sodium hydride (4.98 g, 207.4 mmol; 60% in mineral oil) at 0° C. The reaction mixture was stirred for 1 hour at same temperature. The solution of Iodomethane (27.18 mL, 436.59 mmol) in tetrahydrofuran (10 mL) was added dropwise to the reaction mixture at the same temperature. After the addition, the reaction mixture was warmed to room temperature and stirred overnight. The reaction mixture was poured into water (200 mL) and extracted with ethyl acetate. The resulting organic layer was dried over magnesium sulfate, filtered, and concentrated under reduced pressure. The resulting residue was purified by silica gel column chromatography (n-hexane/ethyl acetate=10/1, v/v) to give the titled compound as yellow oil. (27 g, Yield: 96%)

¹H-NMR (400 MHZ, CDCl₃) δ 7.68 (d, 1H), 7.44 (d, 1H), 2.96 (s, 2H), 1.24 (s, 6H)

### Step 2. (S)-quinuclidin-3-yl((R)-5-bromo-6-fluoro-2,2-dimethyl-2,3-dihydro-1H-inden-1-yl)carbamate

The titled compound was prepared in accordance with the same procedures as in Preparation 1, using 5-bromo-6-fluoro-2,2-dimethyl-2,3-dihydro-1H-inden-1-one prepared in Step 1 as a starting material.

¹H-NMR (400 MHZ, CDCl₃) δ 7.34 (m, 1H), 7.00 (d, 1H), 4.99 (m, 1H), 4.89˜4.77 (m, 2H), 3.29 (m, 1H), 2.96˜2.63 (m, 7H), 2.08 (m, 1H), 1.83 (m, 1H), 1.70 (m, 1H), 1.58 (m, 1H), 1.42 (m, 1H), 1.25 (s, 3H), 0.93 (s, 3H)

### Preparation 3. (S)-quinuclidin-3-yl((R)-5-bromo-2,2,6-trimethyl-2,3-dihydro-1H-inden-1-yl)carbamate

The titled compound was prepared in accordance with the same procedures as in Preparation 2, using 5-bromo-6-methyl-2,3-dihydro-1H-inden-1-one as a starting material.

¹H-NMR (400 MHz, CDCl₃) δ 7.35 (s, 1H), 7.09 (s, 1H), 4.88 (m, 1H), 4.78 (m, 2H), 3.29 (m, 1H), 2.90˜2.63 (m, 7H), 2.38 (s, 3H), 2.08 (m, 1H), 1.83 (m, 1H), 1.70 (m, 1H), 1.59 (m, 1H), 1.42 (m, 1H), 1.24 (s, 3H), 0.93 (s, 3H)

### Preparation 4. (S)-quinuclidin-3-yl((R)-5-bromo-6-ethoxy-2,2-dimethyl-2,3-dihydro-1H-inden-1-yl)carbamate

The titled compound was prepared in accordance with the same procedures as in Preparation 2, using 5-bromo-6-ethoxy-2,3-dihydro-1H-inden-1-one as a starting material.

¹H-NMR (400 MHZ, CDCl₃) δ 7.34 (s, 1H), 6.78 (s, 1H), 4.85˜4.64 (m, 3H), 4.10 (m, 2H), 3.28 (m, 1H), 2.89˜2.65 (m, 7H), 2.08 (m, 1H), 1.83 (m, 1H), 1.70 (m, 1H), 1.58 (m, 1H), 1.46 (m, 3H), 1.42 (m, 1H), 1.28 (s, 3H), 0.94 (s, 3H)

### Preparation 5. ((R)-6-bromo-2,2-dimethyl-1,2,3,4-(S)-quinuclidin-3-yl tetrahydronaphthalen-1-yl)carbamate

The titled compound was prepared in accordance with the same procedures as in Preparation 2, using 6-bromo-3,4-dihydronaphthalen-1(2H)-one as a starting material.

¹H-NMR (400 MHZ, CDCl₃) δ 7.31˜7.27 (m, 2H), 7.17 (d, 1H), 4.75 (m, 2H), 4.58 (m, 1H), 3.27 (m, 1H), 2.97˜2.71 (m, 7H), 2.07 (m, 1H), 1.82 (m, 1H), 1.66 (m, 3H), 1.59 (m, 1H), 1.41 (m, 1H), 1.03 (s, 3H), 0.91 (s, 3H)

### Preparation 6. (S)-quinuclidin-3-yl((R)-6-bromo-7-fluoro-2,2-dimethyl-1,2,3,4-tetrahydronaphthalen-1-yl)carbamate

The titled compound was prepared in accordance with the same procedures as in Preparation 2, using 6-bromo-7-fluoro-3,4-dihydronaphthalen-1(2H)-one as a starting material.

¹H-NMR (400 MHZ, CDCl₃) δ 7.28 (d, 1H), 7.06 (d, 1H), 4.77 (m, 2H), 4.56 (m, 1H), 3.25 (m, 1H), 2.90˜2.70 (m, 7H), 2.08 (m, 1H), 1.81 (m, 1H), 1.68 (m, 3H), 1.58 (m, 1H), 1.40 (m, 1H), 1.01 (s, 3H), 0.89 (s, 3H)

### Example 1. (S)-quinuclidin-3-yl((R)-5-(3-ethylphenyl)-2,2-dimethyl-2,3-dihydro-1H-inden-1-yl)carbamate

A mixture of (S)-quinuclidin-3-yl((R)-5-bromo-2,2-dimethyl-2,3-dihydro-1H-inden-1-yl)carbamate (20 mg, 0.051 mmol) prepared in Preparation 1,3-ethylphenylboronic acid (11.4 mg, 0.076 mmol), potassium 0.153 mmol), and phosphate (32.4 mg, tetrakis(triphenylphosphine)palladium(0) (5.9 mg, 10 mol %) in 1,4-dioxane (1 ml) and water (0.5 ml) was refluxed at 120° C. overnight. The reaction mixture was cooled to room temperature and added to water. The reaction mixture was extracted with ethyl acetate. The organic layer was dried over anhydrous magnesium sulfate, filtered, and concentrated under reduced pressure. The resulting residue was purified by silica gel column chromatography (ethyl acetate/(methanol/ammonia water=1/1)=9/1, v/v) to give the titled compound as a white solid. (18 mg, Yield: 85%)

¹H-NMR (400 MHZ, CDCl₃) δ 7.40 (m, 4H), 7.31 (m, 1H), 7.19 (m, 1H), 4.97 (m, 1H), 4.84 (m, 2H), 3.31 (m, 1H), 2.91˜2.74(m 7H), 2.72 (m, 2H), 2.11(m. 1H), 1.86 (m, 1H), 1.71 (m, 1H), 1.61 (m, 1H), 1.43 (m, 1H), 1.30 (s, 3H), 1.19 (s, 3H), 0.99 (s, 3H)

### Examples 2 to 55

The titled compounds of Examples 2 to 55 were prepared in accordance with the same procedures as in Example 1, using (S)-quinuclidin-3-yl((R)-5-bromo-2,2-dimethyl-2,3-dihydro-1H-inden-1-yl)carbamate prepared in Preparation 1; and the corresponding substituted-boronic acids, respectively.

### Example 2. (S)-quinuclidin-3-yl((R)-5-(3-isopropylphenyl)-2,2-dimethyl-2,3-dihydro-1H-inden-1-yl)carbamate

¹H-NMR (400 MHZ, CDCl₃) δ 7.45˜7.36 (m, 5H), 7.30 (m, 1H), 7.22 (m, 1H), 4.99 (m, 1H), 4.87˜4.81 (m, 2H), 3.29 (m, 1H), 2.98˜2.76 (m, 8H), 2.10 (m, 1H), 1.86 (m, 1H), 1.71 (m, 1H), 1.60 (m, 1H), 1.43 (m, 1H), 1.32 (m, 9H), 0.99 (s, 3H)

### Example 3. (S)-quinuclidin-3-yl((R)-5-(4-butylphenyl)-2,2-dimethyl-2,3-dihydro-1H-inden-1-yl)carbamate

¹H-NMR (400 MHZ, CDCl₃) δ 7.50 (d, 2H), 7.44 (d, 1H), 7.38 (s, 1H), 7.27 (m, 3H), 4.99 (d, 1H), 4.81 (m, 2H), 3.31 (m, 1H), 2.90˜2.68 (m, 7H), 2.64 (m, 2H), 2.09 (m, 1H), 1.95 (m, 1H), 1.72 (m, 1H), 1.68 (m, 2H), 1.58 (m, 3H), 1.42 (m, 1H), 1.30 (s, 3H), 1.00 (t, 3H), 0.98 (s, 3H)

### Example 4. (S)-quinuclidin-3-yl((R)-2,2-dimethyl-5-(4-propylphenyl)-2,3-dihydro-1H-inden-1-yl)carbamate

¹H-NMR (400 MHZ, CDCl₃) δ 7.50 (d, 2H), 7.43 (d, 1H), 7.39 (s, 1H), 7.27 (m, 3H), 4.98 (d, 1H), 4.87˜4.68 (m, 2H), 3.31 (m, 1H), 2.90˜2.74 (m, 7H), 2.63 (m, 2H), 2.10 (m, 1H), 1.81 (m, 1H), 1.61 (m, 3H), 1.59 (m, 1H), 1.41 (m, 1H), 1.30 (s, 3H), 0.98 (m, 6H)

### Example 5. (S)-quinuclidin-3-yl((R)-5-(4-isobutylphenyl)-2,2-dimethyl-2,3-dihydro-1H-inden-1-yl)carbamate

¹H-NMR (400 MHZ, CDCl₃) δ 7.50 (d, 2H), 7.43 (d, 1H), 7.40 (s, 1H), 7.28 (m, 1H), 7.21 (d, 2H), 4.98 (d, 1H), 4.88˜4.66 (m, 2H), 3.31 (m, 1H), 2.90˜2.74 (m, 7H), 2.53 (d, 2H), 2.09 (m, 1H), 1.91 (m, 1H), 1.86 (m, 1H), 1.71 (m, 1H), 1.60 (m, 1H), 1.41 (m, 1H), 1.30 (s, 3H), 0.98 (s, 3H), 0.95 (d, 6H)

### Example 6. (S)-quinuclidin-3-yl((R)-5-(4-(tert-butyl)phenyl)-2,2-dimethyl-2,3-dihydro-1H-inden-1-yl)carbamate

¹H-NMR (400 MHZ, CDCl₃) δ 7.53 (m, 2H), 7.46 (m, 2H), 7.39 (s, 1H), 7.27 (m, 2H), 4.98 (d, 1H), 4.87˜4.70 (m, 2H), 3.30 (m, 1H), 2.91˜2.74 (m, 7H), 2.11 (m, 1H), 1.86 (m, 1H), 1.72 (m, 1H), 1.60 (m, 1H), 1.41 (m, 1H), 1.37 (s, 9H), 1.30 (s, 3H), 0.99 (s, 3H)

### Example 7. (S)-quinuclidin-3-yl((R)-5-(4-ethoxyphenyl)-2,2-dimethyl-2,3-dihydro-1H-inden-1-yl)carbamate

¹H-NMR (400 MHZ, CDCl₃) δ 7.49 (d, 2H), 7.39 (d, 1H), 7.36 (s, 1H), 7.28 (m, 1H), 6.96 (d, 2H), 4.98 (d, 1H), 4.87˜4.68 (m, 2H), 4.08(q, 2H), 3.31 (m, 1H), 2.90˜2.73 (m, 7H), 2.09 (m, 1H), 1.85 (m, 1H), 1.71 (m, 1H), 1.59 (m, 1H), 1.45 (m, 1H), 1.30 (s, 3H), 0.98 (s, 3H)

### Example 8. (S)-quinuclidin-3-yl((R)-5-(4-isobutoxyphenyl)-2,2-dimethyl-2,3-dihydro-1H-inden-1-yl)carbamate

¹H-NMR (400 MHZ, CDCl₃) δ 7.49 (d, 2H), 7.39 (d, 1H), 7.36 (s, 1H), 7.28 (m, 1H), 6.96 (d, 2H), 4.97 (d, 1H), 4.83˜4.64 (m, 2H), 3.77 (d, 2H), 3.30(m 1H), 2.90˜2.74 (m, 7H), 2.11 (m, 1H), 2.10 (m, 1H), 1.85 (m, 1H), 1.71 (m, 1H), 1.59 (m, 1H), 1.45 (m, 1H), 1.30 (s, 3H), 1.05 (d, 6H), 0.98 (s, 3H)

### Example 9. (S)-quinuclidin-3-yl((R)-5-(3,5-dimethyl-4-propoxyphenyl)-2,2-dimethyl-2,3-dihydro-1H-inden-1-yl)carbamate

¹H-NMR (400 MHZ, CDCl₃) δ 7.40 (d, 1H), 7.35 (s, 1H), 7.26 (m, 1H), 7.22 (s, 2H), 4.99 (d, 1H), 4.86˜4.65 (m, 2H), 3.77 (t, 2H), 3.29 (m, 1H), 2.90˜2.73 (m, 7H), 2.34 (s, 6H), 2.05 (m, 1H), 1.87 (m, 3H), 1.71 (m, 1H), 1.59 (m, 1H), 1.43 (m, 1H), 1.29 (s, 3H), 1.10 (t, 3H), 0.98 (s, 3H)

### Example 10. (S)-quinuclidin-3-yl((R)-5-(4-isopropoxy-3,5-dimethylphenyl)-2,2-dimethyl-2,3-dihydro-1H-inden-1-yl)carbamate

¹H-NMR (400 MHZ, CDCl₃) δ 7.40 (d, 1H), 7.37 (s, 1H), 7.27 (m, 1H), 7.22 (s, 2H), 4.98 (d, 1H), 4.86˜4.64 (m, 2H), 4.22 (m, 1H), 3.30 (m, 1H), 2.91˜2.77 (m, 7H), 2.33 (s, 6H), 2.09 (m, 1H), 1.86 (m, 1H), 1.71 (m, 1H), 1.62 (m, 1H), 1.43 (m, 1H), 1.34 (d, 6H), 1.30 (s, 3H), 0.98 (s, 3H)

### Example 11. (S)-quinuclidin-3-yl((R)-5-(3-chloro-4-isopropoxyphenyl)-2,2-dimethyl-2,3-dihydro-1H-inden-1-yl)carbamate

¹H-NMR (400 MHZ, CDCl₃) δ 7.59 (s, 1H), 7.41 (m, 2H), 7.34 (s, 1H), 7.29 (m, 1H), 7.00 (d, 1H), 4.98 (d, 1H), 4.84˜4.67 (m, 2H), 4.58 (m, 1H), 3.30 (m, 1H), 2.90˜2.73 (m, 7H), 2.09 (m, 1H), 1.85 (m, 1H), 1.70 (m, 1H), 1.60 (m, 1H), 1.42 (m, 1H), 1.41 (d, 6H), 1.30 (s, 3H), 0.98 (s, 3H)

### Example 12. (S)-quinuclidin-3-yl((R)-5-(2-fluoro-4-methoxyphenyl)-2,2-dimethyl-2,3-dihydro-1H-inden-1-yl) carbamate

¹H-NMR (400 MHZ, CDCl₃) δ 7.34 (m, 4H), 6.78 (d, 1H), 6.70 (d, 1H), 4.99 (m, 1H), 4.87˜4.65 (m, 2H), 3.85 (s, 3H), 3.30 (m, 1H), 2.91˜2.74 (m, 7H), 2.10 (m, 1H), 1.86 (m, 1H), 1.71 (m, 1H), 1.60 (m, 1H), 1.43 (m, 1H), 1.30 (s, 3H), 0.99 (s, 3H)

### Example 13. (S)-quinuclidin-3-yl((R)-5-(6-(cyclopropylmethoxy)pyridin-3-yl)-2,2-dimethyl-2,3-dihydro-1H-inden-1-yl)carbamate

¹H-NMR (400 MHZ, CDCl₃) δ 8.33 (s, 1H), 7.78 (d, 1H), 7.34 (m, 1H), 7.30 (s, 1H), 7.29 (m, 1H), 6.84 (d, 1H), 4.98 (d, 1H), 4.85 (d, 1H), 4.80˜4.66 (m, 1H), 4.17 (d, 2H), 3.30 (m, 1H), 2.90˜2.75 (m, 7H), 2.09 (m, 1H), 1.85 (m, 1H), 1.71 (m, 1H), 1.59 (m, 1H), 1.42 (m, 1H), 1.30 (s, 3H), 0.98 (s, 3H), 0.66 (m, 2H), 0.36 (m, 2H)

### Example 14. (S)-quinuclidin-3-yl((R)-5-(3-chloro-4-methoxyphenyl)-2,2-dimethyl-2,3-dihydro-1H-inden-1-yl)carbamate

¹H-NMR (400 MHZ, CDCl₃) δ 7.59 (s, 1H), 7.44 (d, 1H), 7.41 (d, 1H), 7.34 (s, 1H), 7.29 (m, 1H), 6.98 (d, 1H), 4.97˜4.80 (m, 2H), 4.79˜4.73 (m, 1H), 3.95 (s, 3H), 3.30 (m, 1H), 2.90˜2.73 (m, 7H), 2.09 (m, 1H), 1.84 (m, 1H), 1.70 (m, 1H), 1.59 (m, 1H), 1.42 (m, 1H), 1.30 (s, 3H), 0.97 (s, 3H)

### Example 15. (S)-quinuclidin-3-yl((R)-5-(3-chloro-4-ethoxyphenyl)-2,2-dimethyl-2,3-dihydro-1H-inden-1-yl)carbamate

¹H-NMR (400 MHZ, CDCl₃) δ 7.59 (s, 1H), 7.42˜7.33 (m, 3H), 7.29 (m, 1H), 6.97 (d, 1H), 4.98˜4.80 (m, 2H), 4.78 (m, 1H), 4.16 (m, 2H), 3.27 (m, 1H), 2.88˜2.73 (m, 7H), 2.05 (m, 1H), 1.85 (m, 1H), 1.71 (m, 1H), 1.59 (m, 1H), 1.50 (t, 3H), 1.42 (m, 1H), 1.30 (s, 3H), 0.97 (s, 3H)

### Example 16. (S)-quinuclidin-3-yl((R)-5-(3-chloro-4-propoxyphenyl)-2,2-dimethyl-2,3-dihydro-1H-inden-1-yl)carbamate

¹H-NMR (400 MHz, CDCl₃) δ 7.59 (s, 1H), 7.41˜7.33 (m, 3H), 7.29 (m, 1H), 6.97 (d, 1H), 4.99˜4.80 (m, 2H), 4.78 (m, 1H), 4.02 (m, 2H), 3.29 (m, 1H), 2.89˜2.73 (m, 7H), 2.08 (m, 1H), 1.92 (m, 2H), 1.85 (m, 1H), 1.69 (m, 1H), 1.59 (m, 1H), 1.42 (m, 1H), 1.30 (s, 3H), 1.08 (t, 3H), 0.97 (s, 3H)

### Example 17. (S)-quinuclidin-3-yl((R)-5-(4-butoxy-3-chlorophenyl)-2,2-dimethyl-2,3-dihydro-1H-inden-1-yl)carbamate

¹H-NMR (400 MHZ, CDCl₃) δ 7.59 (s, 1H), 7.41˜7.33 (m, 3H), 7.29 (m, 1H), 6.97 (d, 1H), 4.99˜4.80 (m, 2H), 4.78 (m, 1H), 4.07 (m, 2H), 3.27 (m, 1H), 2.89˜2.73 (m, 7H), 2.05 (m, 1H), 1.86 (m, 3H), 1.69 (m, 1H), 1.59 (m, 3H), 1.41 (m, 1H), 1.29 (s, 3H), 1.03 (t, 3H), 0.97 (s, 3H)

### Example 18. (S)-quinuclidin-3-yl((R)-5-(3-fluoro-4-methoxyphenyl)-2,2-dimethyl-2,3-dihydro-1H-inden-1-yl)carbamate

¹H-NMR (400 MHZ, CDCl₃) δ 7.35˜7.28 (m, 5H), 7.02 (m, 1H), 4.99˜4.81 (m, 2H), 4.78˜4.70 (m, 1H), 3.93 (s, 3H), 3.29 (m, 1H), 2.89˜2.73 (m, 7H), 2.08 (m, 1H), 1.84 (m, 1H), 1.71 (m, 1H), 1.59 (m, 1H), 1.42 (m, 1H), 1.29 (s, 3H), 0.98 (s, 3H)

### Example 19. (S)-quinuclidin-3-yl((R)-5-(4-ethoxy-3-fluorophenyl)-2,2-dimethyl-2,3-dihydro-1H-inden-1-yl)carbamate

¹H-NMR (400 MHZ, CDCl₃) δ 7.35 (d, 1H), 7.30 (s, 1H), 7.27 (m, 3H), 6.99 (t, 1H), 4.99˜4.81 (m, 2H), 4.72 (m, 1H), 4.15 (m, 2H), 3.28 (m, 1H), 2.89˜2.73 (m, 7H), 2.08 (m, 1H), 1.84 (m, 1H), 1.69 (m, 1H), 1.58 (m, 1H), 1.49 (t, 3H), 1.42 (m, 1H), 1.29 (s, 3H), 0.97 (s, 3H)

### Example 20. (S)-quinuclidin-3-yl((R)-5-(3-fluoro-4-propoxyphenyl)-2,2-dimethyl-2,3-dihydro-1H-inden-1-yl)carbamate

¹H-NMR (400 MHZ, CDCl₃) δ 7.38˜7.25 (m, 5H), 7.00 (t, 1H), 4.99˜4.80 (m, 2H), 4.78 (m, 1H), 4.04 (m, 2H), 3.27 (m, 1H), 2.89˜2.73 (m, 7H), 2.02 (m, 1H), 1.88 (m, 3H), 1.71 (m, 1H), 1.59 (m, 1H), 1.41 (m, 1H), 1.29 (s, 3H), 1.07 (t, 3H), 0.97 (s, 3H)

### Example 21. (S)-quinuclidin-3-yl((R)-5-(3-fluoro-4-isopropoxyphenyl)-2,2-dimethyl-2,3-dihydro-1H-inden-1-yl)carbamate

¹H-NMR (400 MHZ, CDCl₃) δ 7.37 (m, 1H), 7.33 (m, 1H), 7.28 (m, 3H), 7.03 (t, 1H), 4.99˜4.80 (m, 2H), 4.78 (m, 1H), 4.59 (m, 1H), 3.27 (m, 1H), 2.89˜2.73 (m, 7H), 2.09 (m, 1H), 1.84 (m, 1H), 1.70 (m, 1H), 1.59 (m, 1H), 1.41 (m, 1H), 1.40 (d, 6H), 1.29 (s, 3H), 0.97 (s, 3H)

### Example 22. (S)-quinuclidin-3-yl((R)-5-(4-ethoxy-3,5-dimethylphenyl)-2,2-dimethyl-2,3-dihydro-1H-inden-1-yl)carbamate

¹H-NMR (400 MHZ, CDCl₃) δ 7.34 (m, 2H), 7.28 (m, 1H), 7.21 (s, 2H), 4.99˜4.80 (m, 2H), 4.78 (m, 1H), 3.87 (m, 2H), 3.27 (m, 1H), 2.89˜2.72 (m, 7H), 2.34 (s, 6H), 2.08 (m, 1H), 1.84 (m, 1H), 1.69 (m, 1H), 1.57 (m, 1H), 1.46 (m, 3H), 1.42 (m, 1H), 1.29 (s, 3H), 0.97 (s, 3H)

### Example 23. (S)-quinuclidin-3-yl((R)-5-(3-chloro-4-methoxyphenyl)-2,2-dimethyl-2,3-dihydro-1H-inden-1-yl)carbamate

¹H-NMR (400 MHZ, CDCl₃) δ 7.60 (s, 1H), 7.44 (d, 1H), 7.36 (d, 1H), 7.33 (s, 1H), 7.29 (s, 1H), 7.00 (d, 1H), 4.97 (m, 2H), 4.73 (m, 1H), 3.96 (s, 3H), 3.29 (m, 1H), 2.99˜2.73 (m, 7H), 2.10 (m, 1H), 1.84 (m, 1H), 1.71 (m, 1H), 1.58 (m, 1H), 1.42 (m, 1H), 1.29 (s, 3H), 0.94 (s, 3H)

### Example 24. (S)-quinuclidin-3-yl((R)-5-(3-chloro-4-ethoxyphenyl)-2,2-dimethyl-2,3-dihydro-1H-inden-1-yl)carbamate

¹H-NMR (400 MHZ, CDCl₃) δ 7.59 (s, 1H), 7.41˜7.33 (m, 3H), 7.27 (s, 1H), 6.97 (d, 1H), 4.99˜4.81 (m, 2H), 4.79 (m, 1H), 4.15(q, 2H), 3.29 (m, 1H), 2.89˜2.73 (m, 7H), 2.08 (m, 1H), 1.84 (m, 1H), 1.69 (m, 1H), 1.59 (m, 1H), 1.51 (t, 3H), 1.42 (m, 1H), 1.29 (s, 3H), 0.97 (s, 3H)

### Example 25. (S)-quinuclidin-3-yl((R)-5-(3-chloro-4-propoxyphenyl)-2,2-dimethyl-2,3-dihydro-1H-inden-1-yl)carbamate

¹H-NMR (400 MHZ, CDCl₃) δ 7.59 (s, 1H), 7.41˜7.33 (m, 3H), 7.27 (s, 1H), 6.97 (d, 1H), 4.99˜4.81 (m, 2H), 4.79 (m, 1H), 4.05 (t, 2H), 3.29 (m, 1H), 2.89˜2.73 (m, 7H), 2.08 (m, 1H), 1.90 (m, 2H), 1.85 (m, 1H), 1.69 (m, 1H), 1.59 (m, 1H), 1.42 (m, 1H), 1.29 (s, 3H), 1.09 (t, 3H), 0.97 (s, 3H)

### Example 26. (S)-quinuclidin-3-yl((R)-5-(4-butoxy-3-chlorophenyl)-2,2-dimethyl-2,3-dihydro-1H-inden-1-yl)carbamate

¹H-NMR (400 MHZ, CDCl₃) δ 7.59 (s, 1H), 7.41˜7.33 (m, 3H), 7.27 (s, 1H), 6.97 (d, 1H), 4.99˜4.80 (m, 2H), 4.78 (m, 1H), 4.08 (t, 2H), 3.27 (m, 1H), 2.89˜2.73 (m, 7H), 2.08 (m, 1H), 1.85 (m, 3H), 1.69 (m, 1H), 1.59 (m, 3H), 1.41 (m, 1H), 1.29 (s, 3H), 1.00 (t, 3H), 0.98 (s, 3H)

### Example 27. (S)-quinuclidin-3-yl((R)-5-(3-fluoro-4-methoxyphenyl)-2,2-dimethyl-2,3-dihydro-1H-inden-1-yl)carbamate

¹H-NMR (400 MHZ, CDCl₃) δ 7.38˜7.27 (m, 5H), 7.02 (m, 1H), 4.99˜4.79 (m, 2H), 4.73 (m, 1H), 3.93 (s, 3H), 3.27 (m, 1H), 2.89˜2.77 (m, 7H), 2.08 (m, 1H), 1.85 (m, 1H), 1.70 (m, 1H), 1.59 (m, 1H), 1.42 (m, 1H), 1.29 (s, 3H), 0.97 (s, 3H)

### Example 28. (S)-quinuclidin-3-yl((R)-5-(4-ethoxy-3-fluorophenyl)-2,2-dimethyl-2,3-dihydro-1H-inden-1-yl)carbamate

¹H-NMR (400 MHZ, CDCl₃) δ 7.36˜7.30 (m, 2H), 7.26 (m, 3H), 7.01 (m, 1H), 4.99˜4.78 (m, 2H), 4.72 (m, 1H), 4.15(q, 2H), 3.27 (m, 1H), 2.89˜2.73 (m, 7H), 2.09 (br, 1H), 1.84 (m, 1H), 1.70 (m, 1H), 1.60 (m, 1H), 1.49 (t, 3H), 1.42 (m, 1H), 1.29 (s, 3H), 0.97 (s, 3H)

### Example 29. (S)-quinuclidin-3-yl((R)-5-(3-fluoro-4-propoxyphenyl)-2,2-dimethyl-2,3-dihydro-1H-inden-1-yl)carbamate

¹H-NMR (400 MHZ, CDCl₃) δ 7.38 (m, 1H), 7.32 (s, 1H), 7.25 (m, 3H), 6.99 (m, 1H), 4.99˜4.90 (m, 2H), 4.79 (m, 1H), 4.04 (t, 2H), 3.27 (m, 1H), 2.89˜2.73 (m, 7H), 2.05 (br, 1H), 1.85 (m, 3H), 1.70 (m, 1H), 1.59 (m, 1H), 1.42 (m, 1H), 1.29 (s, 3H), 1.09 (t, 3H), 0.97 (s, 3H)

### Example 30. (S)-quinuclidin-3-yl((R)-5-(3-fluoro-4-isopropoxyphenyl)-2,2-dimethyl-2,3-dihydro-1H-inden-1-yl)carbamate

¹H-NMR (400 MHZ, CDCl₃) δ 7.38˜7.32 (m, 2H), 7.27 (m, 3H), 7.03 (t, 1H), 4.99˜4.80 (m, 2H), 4.76 (m, 1H), 4.59 (m, 1H), 3.27 (m, 1H), 2.89˜2.73 (m, 7H), 2.09 (br, 1H), 1.84 (m, 1H), 1.70 (m, 1H), 1.59 (m, 1H), 1.41 (m, 1H), 1.40 (d, 6H), 1.29 (s, 3H), 0.96 (s, 3H)

### Example 31. (S)-quinuclidin-3-yl((R)-5-(4-ethoxy-3,5-dimethylphenyl)-2,2-dimethyl-2,3-dihydro-1H-inden-1-yl)carbamate

¹H-NMR (400 MHZ, CDCl₃) δ 7.37 (m, 2H), 7.25 (m, 1H), 7.21 (s, 2H), 4.99˜4.80 (m, 2H), 4.79 (m, 1H), 3.88(q, 2H), 3.27 (m, 1H), 2.89˜2.72 (m, 7H), 2.34 (s, 6H), 2.09 (br, 1H), 1.84 (m, 1H), 1.70 (m, 1H), 1.59 (m, 1H), 1.41 (t, 3H), 1.40 (m, 1H), 1.29 (s, 3H), 0.97 (s, 3H)

### Example 32. (S)-quinuclidin-3-yl((R)-5-(2-chloro-4-isopropoxyphenyl)-2,2-dimethyl-2,3-dihydro-1H-inden-1-yl)carbamate

¹H-NMR (400 MHz, CDCl₃) δ 7.27 (s, 2H), 7.20 (d, 2H), 7.00 (s, 1H), 6.84 (d, 1H), 4.98 (m, 2H), 4.78 (m, 1H), 4.56 (m, 1H), 3.26 (m, 1H), 2.89˜2.73 (m, 7H), 2.08 (br, 1H), 1.83 (m, 1H), 1.70 (m, 1H), 1.59 (m, 1H), 1.40 (m, 1H), 1.38 (d, 6H), 1.29 (s, 3H), 0.96 (s, 3H)

### Example 33. (S)-quinuclidin-3-yl((R)-5-(4-ethyl-3-fluorophenyl)-2,2-dimethyl-2,3-dihydro-1H-inden-1-yl)carbamate

¹H-NMR (400 MHZ, CDCl₃) δ 7.37 (d, 1H), 7.30 (s, 1H), 7.27˜7.21 (m, 4H), 4.96 (m, 2H), 4.78 (m, 1H), 3.26 (m, 1H), 2.88˜2.72(m, H), 2.69(q, 2H), 2.09 (m, 2H), 1.86 (br, 1H), 1.71 (br, 1H), 1.59 (br, 1H), 1.41 (br, 1H), 1.26 (s, 3H), 1.21 (t, 3H), 0.98 (s, 3H)

### Example 34. (S)-quinuclidin-3-yl((R)-5-(4-isopropoxy-3-methylphenyl)-2,2-dimethyl-2,3-dihydro-1H-inden-1-yl)carbamate

¹H-NMR (400 MHZ, CDCl₃) δ 7.34 (m, 4H), 7.25 (m, 1H), 6.88 (d, 1H), 4.96 (m, 2H), 4.78 (m, 1H), 4.56 (m, 1H), 3.28 (m, 1H), 2.88˜2.73 (m, 7H), 2.27 (s, 3H), 2.07 (br, 1H), 1.84 (m, 1H), 1.68 (m, 1H), 1.59 (m, 1H), 1.41 (m, 1H), 1.39 (d, 6H), 1.29 (s, 3H), 0.98 (s, 3H)

### Example 35. (S)-quinuclidin-3-yl((R)-5-(4-isopropoxy-3-methoxyphenyl)-2,2-dimethyl-2,3-dihydro-1H-inden-1-yl)carbamate

¹H-NMR (400 MHZ, CDCl₃) δ 7.40 (m, 1H), 7.36 (s, 1H), 7.28 (m, 1H), 7.10 (m, 2H), 6.96 (m, 1H), 4.99˜4.81 (m, 2H), 4.79 (m, 1H), 4.57 (m, 1H), 3.93 (s, 3H), 3.28 (m, 1H), 2.89˜2.74 (m, 7H), 2.09 (br, 1H), 1.95 (m, 1H), 1.71 (m, 2H), 1.60 (m, 1H), 1.41 (m, 1H), 1.39 (d, 6H), 1.29 (s, 3H), 0.98 (s, 3H)

### Example 36. (S)-quinuclidin-3-yl((R)-5-(4-isopropoxy-2-methylphenyl)-2,2-dimethyl-2,3-dihydro-1H-inden-1-yl)carbamate

¹H-NMR (400 MHZ, CDCl₃) δ 7.26 (m, 2H), 7.12 (m, 2H), 6.77 (m, 2H), 4.99˜4.82 (m, 2H), 4.81 (m, 1H), 4.58 (m, 1H), 3.29 (m, 1H), 2.91˜2.76 (m, 7H), 2.24 (s, 3H), 2.05 (m, 1H), 1.86 (m, 1H), 1.71 (m, 1H), 1.61 (m, 1H), 1.41 (m, 1H), 1.37 (d, 6H), 1.29 (s, 3H), 0.99 (s, 3H)

### Example 37. (S)-quinuclidin-3-yl((R)-5-(3,5-difluoro-4-isopropoxyphenyl)-2,2-dimethyl-2,3-dihydro-1H-inden-1-yl)carbamate

¹H-NMR (400 MHZ, CDCl₃) δ 7.36˜7.27 (m, 3H), 7.11 (m, 2H), 5.00˜4.89 (m, 2H), 4.79 (m, 1H), 4.47 (m, 1H), 3.29 (m, 1H), 2.89˜2.73 (m, 7H), 2.09 (br, 1H), 1.88 (m, 1H), 1.71 (m, 1H), 1.59 (m, 1H), 1.41 (m, 1H), 1.38 (d, 6H), 1.29 (s, 3H), 0.97 (s, 3H)

### Example 38. (S)-quinuclidin-3-yl((R)-5-(2-chloro-4-(cyclopropylmethoxy)phenyl)-2,2-dimethyl-2,3-dihydro-1H-inden-1-yl)carbamate

¹H-NMR (400 MHZ, CDCl₃) δ 7.27˜7.21 (m, 4H), 7.01 (s, 1H), 6.86 (d, 1H), 5.00˜4.85 (m, 2H), 4.78 (m, 1H), 3.84 (d, 2H), 3.28 (m, 1H), 2.88˜2.73 (m, 7H), 2.09 (br, 1H), 1.89 (m, 1H), 1.69 (m, 1H), 1.59 (m, 1H), 1.42 (m, 1H), 1.29 (s, 3H), 0.99 (s, 3H), 0.68 (d, 2H), 0.38 (d, 2H)

### Example 39. (S)-quinuclidin-3-yl((R)-5-(3-chloro-4-(cyclopropylmethoxy)phenyl)-2,2-dimethyl-2,3-dihydro-1H-inden-1-yl)carbamate

¹H-NMR (400 MHZ, CDCl₃) δ 7.59 (s, 1H), 7.40 (m, 3H), 7.28 (m, 1H), 6.96 (d, 1H), 4.99˜4.81 (m, 2H), 4.79 (m, 1H), 3.93 (d, 2H), 3.27 (m, 1H), 2.89˜2.73 (m, 7H), 2.08 (br, 1H), 1.84 (m, 1H), 1.69 (m, 1H), 1.59 (m, 1H), 1.41 (m, 1H), 1.29 (s, 3H), 0.97 (s, 3H), 0.68 (d, 2H), 0.38 (d, 2H)

### Example 40. (S)-quinuclidin-3-yl((R)-5-(3,5-dichloro-4-isopropoxyphenyl)-2,2-dimethyl-2,3-dihydro-1H-inden-1-yl)carbamate

¹H-NMR (400 MHZ, CDCl₃) δ 7.49 (s, 2H), 7.28 (m, 3H), 5.00˜4.89 (m, 2H), 4.79 (m, 1H), 4.65 (m, 1H), 3.29 (m, 1H), 2.89˜2.73 (m, 7H), 2.09 (br, 1H), 1.88 (m, 1H), 1.70 (m, 1H), 1.59 (m, 1H), 1.41 (m, 1H), 1.39 (d, 6H), 1.29 (s, 3H), 0.96 (s, 3H)

### Example 41. (S)-quinuclidin-3-yl((R)-5-(4-(tert-butoxy)phenyl)-2,2-dimethyl-2,3-dihydro-1H-inden-1-yl)carbamate

¹H-NMR (400 MHZ, CDCl₃) δ 7.46 (d, 2H), 7.42 (d, 1H), 7.37 (s, 1H), 7.28 (m, 1H), 7.04 (d, 2H), 4.99˜4.90 (m, 2H), 4.79 (m, 1H), 3.27 (m, 1H), 2.90˜2.73 (m, 7H), 2.09 (br, 1H), 1.85 (m, 1H), 1.71 (m, 1H), 1.59 (m, 1H), 1.42 (m, 1H), 1.38 (s, 9H), 1.29 (s, 3H), 0.97 (s, 3H)

### Example 42. (S)-quinuclidin-3-yl((R)-5-(4-(tert-butoxy)-2-chlorophenyl)-2,2-dimethyl-2,3-dihydro-1H-inden-1-yl)carbamate

¹H-NMR (400 MHZ, CDCl₃) δ 7.28 (s, 2H), 7.22 (m, 1H), 7.20 (s, 1H), 7.11 (s, 1H), 6.93 (m, 1H), 4.98 (m, 2H), 4.79 (m, 1H), 3.26 (m, 1H), 2.89˜2.77 (m, 7H), 2.09 (m, 1H), 1.84 (m, 1H), 1.70 (m, 1H), 1.59 (m, 1H), 1.41 (m, 1H), 1.40 (s, 9H), 1.29 (s, 3H), 0.99 (s, 3H)

### Example 43. (S)-quinuclidin-3-yl((R)-5-(2-chloro-4-methoxyphenyl)-2,2-dimethyl-2,3-dihydro-1H-inden-1-yl)carbamate

¹H-NMR (400 MHZ, CDCl₃) δ 7.27˜7.21 (m, 4H), 7.02 (s, 1H), 6.86 (m, 1H), 4.96 (m, 2H), 4.79 (m, 1H), 3.84 (s, 3H), 3.26 (m, 1H), 2.89˜2.73 (m, 7H), 2.09 (br, 1H), 1.84 (m, 1H), 1.70 (m, 1H), 1.60 (m, 1H), 1.41 (m, 1H), 1.29 (s, 3H), 0.96 (s, 3H)

### Example 44. (S)-quinuclidin-3-yl((R)-5-(2-chloro-4-ethoxyphenyl)-2,2-dimethyl-2,3-dihydro-1H-inden-1-yl)carbamate

¹H-NMR (400 MHZ, CDCl₃) δ 7.27˜7.21 (m, 4H), 7.00 (s, 1H), 6.84 (m, 1H), 5.00˜4.93 (m, 2H), 4.80 (m, 1H), 4.05(q, 2H), 3.27 (m, 1H), 2.89˜2.73 (m, 7H), 2.09 (br, 1H), 1.89 (m, 1H), 1.70 (m, 1H), 1.59 (m, 1H), 1.44 (t, 3H), 1.42 (m, 1H), 1.30 (s, 3H), 0.99 (s, 3H)

### Example 45. (S)-quinuclidin-3-yl((R)-5-(3-chloro-4-methylphenyl)-2,2-dimethyl-2,3-dihydro-1H-inden-1-yl)carbamate

¹H-NMR (400 MHZ, CDCl₃) δ 7.56 (s, 1H), 7.35 (m, 3H), 7.27 (m, 2H), 5.00˜4.91 (m, 2H), 4.80 (m, 1H), 3.28 (m, 1H), 2.89˜2.73 (m, 7H), 2.41 (s, 3H), 2.09 (br, 1H), 1.86 (m, 1H), 1.71 (m, 1H), 1.59 (m, 1H), 1.41 (m, 1H), 1.29 (s, 3H), 0.96 (s, 3H)

### Example 46. (S)-quinuclidin-3-yl((R)-5-(4-ethoxy-3-methylphenyl)-2,2-dimethyl-2,3-dihydro-1H-inden-1-yl)carbamate

¹H-NMR (400 MHZ, CDCl₃) δ 7.37 (m, 4H), 7.25 (m, 1H), 6.86 (d, 1H), 4.98˜4.90 (m, 2H), 4.79 (m, 1H), 4.07(q, 2H), 3.27 (m, 1H), 2.88˜2.73 (m, 7H), 2.29 (s, 3H), 2.09 (br, 1H), 1.84 (m, 1H), 1.70 (m, 1H), 1.59 (m, 1H), 1.44 (t, 3H), 1.41 (m, 1H), 1.29 (s, 3H), 0.98 (s, 3H)

### Example 47. (S)-quinuclidin-3-yl((R)-2,2-dimethyl-5-(3-methyl-4-propoxyphenyl)-2,3-dihydro-1H-inden-1-yl) carbamate

¹H-NMR (400 MHZ, CDCl₃) δ 7.37 (m, 4H), 7.25 (m, 1H), 6.86 (d, 1H), 4.98˜4.91 (m, 2H), 4.79 (m, 1H), 3.97 (t, 2H), 3.27 (m, 1H), 2.88˜2.73 (m, 7H), 2.29 (s, 3H), 2.09 (br, 1H), 1.86 (m, 3H), 1.69 (m, 1H), 1.59 (m, 1H), 1.41 (m, 1H), 1.29 (s, 3H), 1.08 (t, 3H), 0.98 (s, 3H)

### Example 48. (S)-quinuclidin-3-yl((R)-5-(4-(difluoromethoxy)-3-methylphenyl)-2,2-dimethyl-2,3-dihydro-1H-inden-1-yl)carbamate

¹H-NMR (400 MHZ, CDCl₃) δ 7.42 (s, 1H), 7.36 (m, 3H), 7.29 (m, 1H), 7.12 (d, 1H), 6.72˜6.35 (t, 1H), 4.99˜4.90 (m, 1H), 4.80 (m, 1H), 3.27 (m, 1H), 2.90˜2.74 (m, 7H), 2.35 (s, 3H), 2.09 (br, 1H), 1.84 (m, 1H), 1.70 (m, 1H), 1.59 (m, 1H), 1.42 (m, 1H), 1.29 (s, 3H), 0.97 (s, 3H)

### Example 49. (S)-quinuclidin-3-yl((R)-5-(3-fluoro-5-isopropoxyphenyl)-2,2-dimethyl-2,3-dihydro-1H-inden-1-yl)carbamate

¹H-NMR (400 MHZ, CDCl₃) δ 7.38 (d, 1H), 7.35 (s, 1H), 7.29 (m, 1H), 6.88 (s, 1H), 6.83 (d, 1H), 6.57 (d, 1H), 5.00˜4.89 (m, 2H), 4.80 (m, 1H), 3.27 (m, 1H), 2.89˜2.73 (m, 7H), 2.09 (br, 1H), 1.84 (m, 1H), 1.71 (m, 1H), 1.59 (m, 1H), 1.42 (m, 1H), 1.37 (d, 6H), 1.29 (s, 3H), 0.96 (s, 3H)

### Example 50. (S)-quinuclidin-3-yl((R)-5-(3-fluoro-5-isobutoxyphenyl)-2,2-dimethyl-2,3-dihydro-1H-inden-1-yl)carbamate

¹H-NMR (400 MHZ, CDCl₃) δ 7.40 (d, 1H), 7.35 (s, 1H), 7.27 (m, 1H), 6.89 (s, 1H), 6.86 (d, 1H), 6.58 (m, 1H), 5.00˜4.91 (m, 2H), 4.79 (m, 1H), 3.76 (d, 2H), 3.27 (m, 1H), 2.90˜2.73 (m, 7H), 2.10 (m, 1H), 2.09 (m, 1H), 1.84 (m, 1H), 1.69 (m, 1H), 1.59 (m, 1H), 1.42 (m, 1H), 1.29 (s, 3H), 1.05 (d, 6H), 0.96 (s, 3H)

### Example 51. (S)-quinuclidin-3-yl((R)-5-(3-fluoro-5-(2,2,2-trifluoroethoxy)phenyl)-2,2-dimethyl-2,3-dihydro-1H-inden-1-yl) carbamate

¹H-NMR (400 MHZ, CDCl₃) δ 7.39 (d, 1H), 7.32 (s, 1H), 7.29 (m, 1H), 6.96 (m, 1H), 6.94 (s, 1H), 6.65 (m, 1H), 5.00˜4.95 (m, 2H), 4.80 (m, 1H), 4.41(q, 2H), 3.28 (m, 1H), 2.88˜2.74 (m, 7H), 2.09 (br, 1H), 1.85 (m, 1H), 1.70 (m, 1H), 1.59 (m, 1H), 1.42 (m, 1H), 1.29 (s, 3H), 0.97 (s, 3H)

### Example 52. (S)-quinuclidin-3-yl((R)-5-(4-chloro-3-isopropoxyphenyl)-2,2-dimethyl-2,3-dihydro-1H-inden-1-yl)carbamate

¹H-NMR (400 MHZ, CDCl₃) δ 7.40 (m, 2H), 7.33 (s, 1H), 7.28 (m, 1H), 7.12 (s, 1H), 7.06 (m, 1H), 4.99˜4.81 (m, 2H), 4.80 (m, 1H), 4.65 (m, 1H), 3.28 (m, 1H), 2.88˜2.73 (m, 7H), 2.08 (br, 1H), 1.84 (m, 1H), 1.70 (m, 1H), 1.59 (m, 1H), 1.42 (m, 1H), 1.42 (d, 6H), 1.29 (s, 3H), 0.98 (s, 3H)

### Example 53. (S)-quinuclidin-3-yl((R)-5-(3-chloro-5-isobutoxyphenyl)-2,2-dimethyl-2,3-dihydro-1H-inden-1-yl)carbamate

¹H-NMR (400 MHZ, CDCl₃) δ 7.40 (m, 1H), 7.35 (s, 1H), 7.29 (m, 1H), 7.10 (s, 1H), 6.98 (s, 1H), 6.86 (s, 1H), 5.00˜4.94 (m, 2H), 4.80 (m, 1H), 3.75 (d, 2H), 3.27 (m, 1H), 2.88˜2.73 (m, 7H), 2.10 (m, 2H), 1.84 (m, 1H), 1.68 (m, 1H), 1.59 (m, 1H), 1.42 (m, 1H), 1.29 (s, 3H), 1.05 (d, 6H), 0.97 (s, 3H)

### Example 54. (S)-quinuclidin-3-yl((R)-5-(3-chloro-5-isopropoxyphenyl)-2,2-dimethyl-2,3-dihydro-1H-inden-1-yl)carbamate

¹H-NMR (400 MHZ, CDCl₃) δ 7.37 (d, 1H), 7.35 (s, 1H), 7.29 (m, 1H), 7.12 (s, 1H), 6.96 (s, 1H), 6.84 (s, 1H), 5.00˜4.90 (m, 2H), 4.80 (m, 1H), 4.59 (m, 1H), 3.27 (m, 1H), 2.90˜2.73 (m, 7H), 2.09 (br, 1H) 1.85 (m, 1H), 1.69 (m, 1H), 1.59 (m, 1H), 1.42 (m, 1H), 1.36 (d, 6H), 1.29 (s, 3H), 0.96 (s, 3H)

### Example 55. (S)-quinuclidin-3-yl((R)-5-(2-chloro-4-(2,2,2-trifluoroethoxy)phenyl)-2,2-dimethyl-2,3-dihydro-1H-inden-1-yl)carbamate

¹H-NMR (400 MHZ, CDCl₃) δ 7.32 (m, 2H), 7.29˜7.24 (m, 2H), 7.19˜7.17 (m, 2H), 5.02˜4.95 (m, 2H), 4.80 (m, 1H), 3.28 (m, 1H), 2.89˜2.74 (m, 7H), 2.09 (br, 1H), 1.86 (m, 1H), 1.69 (m, 1H), 1.59 (m, 1H), 1.42 (m, 1H), 1.31 (s, 3H), 0.99 (s, 3H)

### Examples 56 to 114

The titled compounds of Examples 56 to 114 were prepared in accordance with the same procedures as in Example 1, using (S)-quinuclidin-3-yl((R)-5-bromo-6-fluoro-2,2-dimethyl-2,3-dihydro-1H-inden-1-yl)carbamate prepared in Preparation 2; and the corresponding substituted-phenylboronic acids, respectively.

### Example 56. (S)-quinuclidin-3-yl((R)-5-(4-ethoxyphenyl)-6-fluoro-2,2-dimethyl-2,3-dihydro-1H-inden-1-yl)carbamate

¹H-NMR (400 MHZ, CDCl₃) δ 7.45 (d, 2H), 7.18 (d, 1H), 7.00 (d, 1H), 6.97 (d, 2H), 4.98˜4.92 (m, 2H), 4.80 (m, 1H), 4.09 (m, 2H), 3.29 (m, 1H), 2.90 (m, 2H), 2.81˜2.68 (m, 5H), 2.09 (m, 1H), 1.85 (m, 1H), 1.70 (m, 1H), 1.59 (m, 1H), 1.45 (t, 3H), 1.42 (m, 1H), 1.29 (s, 3H), 0.97 (s, 3H)

### Example 57. (S)-quinuclidin-3-yl((R)-6-fluoro-5-(4-isobutoxyphenyl)-2,2-dimethyl-2,3-dihydro-1H-inden-1-yl)carbamate

¹H-NMR (400 MHZ, CDCl₃) δ 7.44 (d, 2H), 7.18 (d, 1H), 7.00 (d, 1H), 6.97 (d, 2H), 4.96˜4.81 (m, 2H), 4.78 (m, 1H), 3.77 (d, 2H), 3.28 (m, 1H), 2.89˜2.82 (m, 2H), 2.79˜2.68 (m, 5H), 2.11 (m, 2H), 1.85 (m, 1H), 1.71 (m, 1H), 1.59 (m, 1H), 1.42 (m, 1H), 1.29 (s, 3H), 1.05 (d, 6H), 0.97 (s, 3H)

### Example 58. (S)-quinuclidin-3-yl((R)-5-(4-ethylphenyl)-6-fluoro-2,2-dimethyl-2,3-dihydro-1H-inden-1-yl)carbamate

¹H-NMR (400 MHZ, CDCl₃) δ 7.44 (m, 2H), 7.28 (m, 2H), 7.21 (d, 1H), 7.02 (d, 1H), 4.97˜4.81 (m, 2H), 4.78 (m, 1H), 3.28 (m, 1H), 2.90˜2.71 (m, 7H), 2.68 (m, 2H), 2.09 (m, 1H), 1.84 (m, 1H), 1.69 (m, 1H), 1.58 (m, 1H), 1.41 (t, 3H), 1.30 (s, 3H), 0.98 (s, 3H)

### Example 59. (S)-quinuclidin-3-yl((R)-6-fluoro-5-(4-isopropylphenyl)-2,2-dimethyl-2,3-dihydro-1H-inden-1-yl)carbamate

¹H-NMR (400 MHZ, CDCl₃) δ 7.45 (m, 2H), 7.30 (m, 2H), 7.20 (d, 1H), 7.02 (d, 1H), 4.99˜4.81 (m, 2H), 4.78 (m, 1H), 3.29 (m, 1H), 2.97 (m, 1H), 2.92˜2.68 (m, 7H), 2.09 (m, 1H), 1.85 (m, 1H), 1.70 (m, 1H), 1.58 (m, 1H), 1.44 (m, 1H), 1.25 (m, 9H), 0.97 (s, 3H)

### Example 60. (S)-quinuclidin-3-yl((R)-5-(4-butylphenyl)-6-fluoro-2,2-dimethyl-2,3-dihydro-1H-inden-1-yl)carbamate

¹H-NMR (400 MHZ, CDCl₃) δ 7.43 (m, 2H), 7.27 (m, 2H), 7.21 (d, 1H), 7.02 (d, 1H), 4.99˜4.81 (m, 2H), 4.79 (m, 1H), 3.29 (m, 1H), 2.88 (m, 2H), 2.78 (m, 5H), 2.66 (m, 2H), 2.09 (m, 1H), 1.85 (m, 1H), 1.70˜1.60 (m, 4H), 1.42 (m, 3H), 1.30 (s, 3H), 0.97 (m, 6H)

### Example 61. (S)-quinuclidin-3-yl((R)-6-fluoro-2,2-dimethyl-5-(4-propylphenyl)-2,3-dihydro-1H-inden-1-yl)carbamate

¹H-NMR (400 MHZ, CDCl₃) δ 7.44 (m, 2H), 7.27 (m, 2H), 7.22 (m, 1H), 7.02 (d, 1H), 4.97 (m, 2H), 4.79 (m, 1H), 3.29 (m, 1H), 2.88 (m, 2H), 2.76 (m, 5H), 2.63 (m, 2H), 2.08 (m, 1H), 1.85 (m, 1H), 1.71 (m, 3H), 1.58 (m, 1H), 1.42 (m, 1H), 1.30 (s, 3H), 0.97 (m, 6H)

### Example 62. (S)-quinuclidin-3-yl((R)-6-fluoro-5-(4-isobutylphenyl)-2,2-dimethyl-2,3-dihydro-1H-inden-1-yl)carbamate

¹H-NMR (400 MHZ, CDCl₃) δ 7.43 (d, 2H), 7.21 (d, 3H), 7.02 (d, 1H), 4.99˜4.92 (m, 2H), 4.79 (m, 1H), 3.28 (m, 1H), 2.90˜2.72 (m, 2H), 2.68 (m, 5H), 2.53 (d, 2H), 2.09 (m, 1H), 1.93 (m, 1H), 1.87 (m, 1H), 1.70 (m, 1H), 1.59 (m, 1H), 1.42 (m, 1H), 1.29 (s, 3H), 0.97 (s, 3H), 0.94 (d, 6H)

### Example 63. (S)-quinuclidin-3-yl((R)-6-fluoro-5-(4-methoxy-3,5-dimethylphenyl)-2,2-dimethyl-2,3-dihydro-1H-inden-1-yl)carbamate

¹H-NMR (400 MHZ, CDCl₃) δ 7.17 (m, 3H), 7.00 (d, 1H), 4.99˜4.90 (m, 2H), 4.80 (m, 1H), 3.76 (s, 3H), 3.26 (m, 1H), 2.93 (m, 2H), 2.78 (m, 5H), 2.34 (s, 6H), 2.09 (m, 1H), 1.84 (m, 1H), 1.70 (m, 1H), 1.60 (m, 1H), 1.43 (m, 1H), 1.29 (s, 3H), 0.97 (s, 3H)

### Example 64. (S)-quinuclidin-3-yl((R)-5-(3,5-dimethyl-4-propoxyphenyl)-6-fluoro-2,2-dimethyl-2,3-dihydro-1H-inden-1-yl)carbamate

¹H-NMR (400 MHZ, CDCl₃) δ 7.17 (m, 3H), 6.99 (d, 1H), 4.96˜4.81 (m, 2H), 4.79 (m, 1H), 3.76 (m, 2H), 3.26 (m, 1H), 2.91 (m, 2H), 2.81˜2.67 (m, 5H), 2.33 (s, 6H), 2.09 (m, 1H), 1.85 (m, 3H), 1.71 (m, 1H), 1.60 (m, 1H), 1.42 (m, 1H), 1.30 (s, 3H), 1.10 (t, 3H), 0.97 (s, 3H)

### Example 65. (S)-quinuclidin-3-yl((R)-6-fluoro-5-(4-isopropoxy-3,5-dimethylphenyl)-2,2-dimethyl-2,3-dihydro-1H-inden-1-yl)carbamate

¹H-NMR (400 MHZ, CDCl₃) δ 7.20˜7.17 (m, 3H), 6.99 (d, 1H), 4.95 (m, 2H), 4.79 (m, 1H), 4.21 (m, 1H), 3.29 (m, 1H), 2.92 (m, 2H), 2.81˜2.67 (m, 5H), 2.32 (s, 6H), 2.09 (m, 1H), 1.85 (m, 1H), 1.70 (m, 1H), 1.58 (m, 1H), 1.42 (m, 1H), 1.29 (d, 6H), 1.25 (s, 3H), 0.97 (s, 3H)

### Example 66. (S)-quinuclidin-3-yl((R)-5-(3-chloro-4-isopropoxyphenyl)-6-fluoro-2,2-dimethyl-2,3-dihydro-1H-inden-1-yl)carbamate

¹H-NMR (400 MHZ, CDCl₃) δ 7.54 (s, 1H), 7.36 (d, 1H), 7.17 (d, 1H), 7.01 (m, 2H), 4.96 (m, 2H), 4.78 (m, 1H), 4.60 (m, 1H), 3.29 (m, 1H), 2.89 (m, 2H), 2.79 (m, 5H), 2.09 (m, 1H), 1.86 (m, 1H), 1.71 (m, 1H), 1.60 (m, 1H), 1.42 (m, 1H), 1.40 (d, 6H), 1.28 (s, 3H), 0.97 (s, 3H)

### Example 67. (S)-quinuclidin-3-yl((R)-6-fluoro-5-(2-fluoro-4-methoxyphenyl)-2,2-dimethyl-2,3-dihydro-1H-inden-1-yl)carbamate

¹H-NMR (400 MHZ, CDCl₃) δ 7.27 (m, 1H), 7.13 (d, 1H), 7.02 (d, 1H), 6.76 (m, 2H), 4.97 (m, 2H), 4.79 (m, 1H), 3.84 (s, 3H), 3.29 (m, 1H), 2.80 (m, 2H), 2.71 (m, 5H), 2.08 (m, 1H), 1.84 (m, 1H), 1.69 (m, 1H), 1.59 (m, 1H), 1.42 (m, 1H), 1.27 (s, 3H), 0.97 (s, 3H)

### Example 68. (S)-quinuclidin-3-yl((R)-6-fluoro-5-(4-isopropoxyphenyl)-2,2-dimethyl-2,3-dihydro-1H-inden-1-yl)carbamate

¹H-NMR (400 MHZ, CDCl₃) δ 7.44 (d, 2H), 7.19 (d, 1H), 7.00 (d, 1H), 6.96 (d, 2H), 4.99˜4.81 (m, 2H), 4.78 (m, 1H), 4.59 (m, 1H), 3.30 (m, 1H), 2.89 (m, 2H), 2.72 (m, 5H), 2.08 (m, 1H), 1.84 (m, 1H), 1.71 (m, 1H), 1.60 (m, 1H), 1.42 (m, 1H), 1.32 (d, 6H), 1.27 (s, 3H), 0.97 (s, 3H)

### Example 69. (S)-quinuclidin-3-yl((R)-6-fluoro-2,2-dimethyl-5-(4-propoxyphenyl)-2,3-dihydro-1H-inden-1-yl)carbamate

¹H-NMR (400 MHZ, CDCl₃) δ 7.45 (d, 2H), 7.19 (d, 1H), 7.00 (d, 1H), 6.98 (d, 2H), 4.98˜4.79 (m, 2H), 4.67 (m, 1H), 3.98 (m, 2H), 3.28 (m, 1H), 2.89 (m, 2H), 2.72 (m, 5H), 2.09 (m, 1H), 1.83 (m, 3H), 1.73 (m, 1H), 1.59 (m, 1H), 1.42 (m, 1H), 1.27 (s, 3H), 1.10 (t, 3H), 0.97 (s, 3H)

### Example 70. (S)-quinuclidin-3-yl((R)-5-(4-butoxyphenyl)-6-fluoro-2,2-dimethyl-2,3-dihydro-1H-inden-1-yl)carbamate

¹H-NMR (400 MHZ, CDCl₃) δ 7.45 (d, 2H), 7.19 (d, 1H), 7.00 (d, 1H), 6.97 (d, 2H), 4.98˜4.92 (m, 2H), 4.79 (m, 1H), 4.01 (m, 2H), 3.27 (m, 1H), 2.89 (m, 2H), 2.78 (m, 5H), 2.08 (m, 1H), 1.85 (m, 3H), 1.71 (m, 1H), 1.59 (m, 1H), 1.56 (m, 2H), 1.42 (m, 1H), 1.29 (s, 3H), 0.99 (m, 6H)

### Example 71. (S)-quinuclidin-3-yl((R)-5-(3-ethylphenyl)-6-fluoro-2,2-dimethyl-2,3-dihydro-1H-inden-1-yl)carbamate

¹H-NMR (400 MHZ, CDCl₃) δ 7.35 (m, 3H), 7.22 (m, 2H), 7.03 (d, 1H), 5.00˜4.79 (m, 2H), 4.69 (m, 1H), 3.30 (m, 1H), 2.80 (m, 2H), 2.78 (m, 5H), 2.71 (m, 2H), 2.09 (m, 1H), 1.84 (m, 1H), 1.70 (m, 1H), 1.59 (m, 1H), 1.43 (m, 1H), 1.30 (t, 3H), 1.29 (s, 3H), 0.98 (s, 3H)

### Example 72. (S)-quinuclidin-3-yl((R)-6-fluoro-2,2-dimethyl-5-(3-propylphenyl)-2,3-dihydro-1H-inden-1-yl)carbamate

¹H-NMR (400 MHZ, CDCl₃) δ 7.35 (m, 3H), 7.21 (m, 2H), 7.02 (d, 1H), 5.00˜4.79 (m, 2H), 4.78 (m, 1H), 3.30 (m, 1H), 2.81 (m, 2H), 2.73 (m, 5H), 2.63 (m, 2H), 2.09 (m, 1H), 1.87 (m, 1H), 1.62 (m, 3H), 1.58 (m, 1H), 1.44 (m, 1H), 1.30 (s, 3H), 0.97 (m, 6H)

### Example 73. (S)-quinuclidin-3-yl((R)-6-fluoro-5-(3-isopropylphenyl)-2,2-dimethyl-2,3-dihydro-1H-inden-1-yl)carbamate

¹H-NMR (400 MHZ, CDCl₃) δ 7.36 (m, 3H), 7.23 (m, 2H), 7.03 (d, 1H), 5.00˜4.80 (m, 2H), 4.74 (m, 1H), 3.30 (m, 1H), 2.97 (m, 1H), 2.81 (m, 2H), 2.77 (m, 5H), 2.09 (m, 1H), 1.86 (m, 1H), 1.71 (m, 1H), 1.60 (m, 1H), 1.40 (m, 1H), 1.30 (m, 9H), 0.98 (s, 3H)

### Example 74. (S)-quinuclidin-3-yl((R)-6-fluoro-5-(3-isobutylphenyl)-2,2-dimethyl-2,3-dihydro-1H-inden-1-yl)carbamate

¹H-NMR (400 MHZ, CDCl₃) δ 7.34 (d, 2H), 7.30 (s, 1H), 7.21 (d, 1H), 7.15 (m, 1H), 7.02 (d, 1H), 5.00˜4.72 (m, 2H), 4.70 (m, 1H), 3.31 (m, 1H), 2.90 (m, 2H), 2.78 (m, 5H), 2.54 (d, 2H), 2.09 (m, 1H), 1.91 (m, 1H), 1.87 (m, 1H), 1.71 (m, 1H), 1.61 (m, 1H), 1.42 (m, 1H), 1.29 (s, 3H), 0.99 (s, 3H), 0.91 (d, 6H)

### Example 75. (S)-quinuclidin-3-yl((R)-6-fluoro-5-(3-methoxyphenyl)-2,2-dimethyl-2,3-dihydro-1H-inden-1-yl)carbamate

¹H-NMR (400 MHZ, CDCl₃) δ 7.36 (m, 1H), 7.22 (d, 1H), 7.11 (d, 1H), 7.09 (m, 2H), 6.92 (m, 1H), 5.00˜4.86 (m, 2H), 4.80˜4.74 (m, 1H), 3.86 (s, 3H), 3.30 (m, 1H), 2.85 (m, 2H), 2.76 (m, 5H), 2.09 (m, 1H), 1.86 (m, 1H), 1.71 (m, 1H), 1.59 (m, 1H), 1.42 (m, 1H), 1.29 (s, 3H), 0.98 (s, 3H)

### Example 76. (S)-quinuclidin-3-yl((R)-5-(3-ethoxyphenyl)-6-fluoro-2,2-dimethyl-2,3-dihydro-1H-inden-1-yl)carbamate

¹H-NMR (400 MHZ, CDCl₃) δ 7.34 (t, 1H), 7.21 (d, 1H), 7.10 (m, 1H), 7.06 (m, 2H), 6.91 (m, 1H), 5.00˜4.80 (m, 2H), 4.79˜4.70 (m, 1H), 4.10 (m, 2H), 3.30 (m, 1H), 2.89 (m, 2H), 2.76 (m, 5H), 2.09 (m, 1H), 1.85 (m, 1H), 1.71 (m, 1H), 1.59 (m, 1H), 1.45 (m, 1H). 1.40 (t, 3H), 1.29 (s, 3H), 0.98 (s, 3H)

### Example 77. (S)-quinuclidin-3-yl((R)-6-fluoro-2,2-dimethyl-5-(3-propoxyphenyl)-2,3-dihydro-1H-inden-1-yl)carbamate

¹H-NMR (400 MHZ, CDCl₃) δ 7.34 (t, 1H), 7.22 (d, 1H), 7.09˜7.01 (m, 3H), 6.89 (m, 1H), 5.00˜4.81 (m, 2H), 4.78 (m, 1H), 3.98 (m, 2H), 3.29 (m, 1H), 2.89 (m, 2H), 2.78 (m, 5H), 2.09 (m, 1H), 1.84 (m, 3H), 1.70 (m, 1H), 1.59 (m, 1H), 1.41 (m, 1H), 1.29 (s, 3H), 1.05 (t, 3H), 0.97(s, 3H Example 78. (S)-quinuclidin-3-yl((R)-6-fluoro-5-(3-isobutoxyphenyl)-2,2-dimethyl-2,3-dihydro-1H-inden-1-yl)carbamate

¹H-NMR (400 MHZ, CDCl₃) δ 7.34 (t, 1H), 7.22 (d, 1H), 7.10˜7.01 (m, 3H), 6.90 (m, 1H), 5.00˜4.90 (m, 2H), 4.79 (m, 1H), 3.78 (d, 2H), 3.30 (m, 1H), 2.89 (m, 2H), 2.76 (m, 5H), 2.08 (m, 2H), 1.84 (m, 1H), 1.71 (m, 1H), 1.60 (m, 1H), 1.42 (m, 1H), 1.30 (s, 3H), 1.05 (d, 6H), 0.98 (s, 3H)

### Example 79. (S)-quinuclidin-3-yl((R)-5-(3-butoxyphenyl)-6-fluoro-2,2-dimethyl-2,3-dihydro-1H-inden-1-yl)carbamate

¹H-NMR (400 MHZ, CDCl₃) δ 7.34 (t, 1H), 7.21 (d, 1H), 7.10˜7.01 (m, 3H), 6.89 (m, 1H), 5.00˜4.90 (m, 2H), 4.81˜4.79 (m, 1H), 4.03 (m, 2H), 3.28 (m, 1H), 2.89 (m, 2H), 2.76 (m, 5H), 2.09 (m, 1H), 1.83 (m, 1H), 1.78 (m, 2H), 1.70 (m, 1H), 1.59 (m, 1H), 1.56 (m, 2H), 1.48 (m, 1H), 1.29 (s, 3H), 0.98 (m, 6H)

### Example 80. (S)-quinuclidin-3-yl((R)-5-(4-ethoxy-3,5-dimethylphenyl)-6-fluoro-2,2-dimethyl-2,3-dihydro-1H-inden-1-yl)carbamate

¹H-NMR (400 MHZ, CDCl₃) δ 7.17 (s, 3H), 7.00 (d, 1H), 4.99˜4.80 (m, 2H), 4.78 (m, 1H), 3.89(q, 2H), 3.29 (m, 1H), 2.89 (m, 2H), 2.75 (m, 5H), 2.33 (s, 6H), 2.09 (m, 1H), 1.85 (m, 1H), 1.70 (m, 1H), 1.60 (m, 1H), 1.45 (m, 4H), 1.29 (s, 3H), 0.98 (s, 3H)

### Example 81. (S)-quinuclidin-3-yl((R)-6-fluoro-5-(3-isopropoxyphenyl)-2,2-dimethyl-2,3-dihydro-1H-inden-1-yl)carbamate

¹H-NMR (400 MHZ, CDCl₃) δ 7.33 (t, 1H), 7.21 (d, 1H), 7.09˜7.01 (m, 3H), 6.88 (m, 1H), 5.00˜4.81 (m, 2H), 4.79 (m, 1H), 4.60 (m, 1H), 3.29 (m, 1H), 2.81 (m, 2H), 2.76 (m, 5H), 2.08 (m, 1H), 1.88 (m, 1H), 1.70 (m, 1H), 1.59 (m, 1H), 1.42 (m, 1H), 1.37 (d, 6H), 1.29 (s, 3H), 0.98 (s, 3H)

### Example 82. (S)-quinuclidin-3-yl((R)-6-fluoro-5-(4-methoxyphenyl)-2,2-dimethyl-2,3-dihydro-1H-inden-1-yl)carbamate

¹H-NMR (400 MHZ, CDCl₃) δ 7.46 (d, 2H), 7.18 (d, 1H), 7.00 (m, 1H), 6.96 (d, 2H), 4.99˜4.88 (m, 2H), 4.79 (m, 1H), 3.86 (s, 3H), 3.29 (m, 1H), 2.85 (m, 2H), 2.76 (m, 5H), 2.09 (m, 1H), 1.84 (m, 1H), 1.70 (m, 1H), 1.59 (m, 1H), 1.42 (m, 1H), 1.30 (s, 3H), 0.97 (s, 3H)

### Example 83. (S)-quinuclidin-3-yl((R)-6-fluoro-5-(3-fluoro-4-isopropoxyphenyl)-2,2-dimethyl-2,3-dihydro-1H-inden-1-yl)carbamate

¹H-NMR (400 MHZ, CDCl₃) δ 7.28 (m, 1H), 7.19 (m, 2H). 7.05 (m, 1H), 7.01 (d, 1H), 4.99˜4.89 (m, 2H), 4.79 (m, 1H), 3.29 (m, 1H), 2.89 (m, 2H), 2.76 (m, 5H), 2.08 (m, 1H), 1.84 (m, 1H), 1.70 (m, 1H), 1.59 (m, 1H), 1.41 (m, 1H), 1.40 (d, 6H), 1.29 (s, 3H), 0.97 (s, 3H)

### Example 84. (S)-quinuclidin-3-yl((R)-5-(3-chloro-4-methylphenyl)-6-fluoro-2,2-dimethyl-2,3-dihydro-1H-inden-1-yl)carbamate

¹H-NMR (400 MHZ, CDCl₃) δ 7.51 (s, 1H), 7.31˜7.27 (m, 2H), 7.18 (d, 1H), 7.02 (d, 1H), 4.99˜4.93 (m, 2H), 4.80 (m, 1H), 3.27 (m, 1H), 2.80 (m, 2H), 2.75 (m, 5H), 2.41 (s, 3H), 2.09 (br, 1H), 1.84 (m, 1H), 1.70 (m, 1H), 1.59 (m, 1H), 1.40 (m, 1H), 1.29 (s, 3H), 0.96 (s, 3H)

### Example 85. (S)-quinuclidin-3-yl((R)-5-(3-chloro-4-methoxyphenyl)-6-fluoro-2,2-dimethyl-2,3-dihydro-1H-inden-1-yl)carbamate

¹H-NMR (400 MHZ, CDCl₃) δ 7.54 (s, 1H), 7.40 (d, 1H), 7.17 (d, 1H), 7.00 (t, 2H), 4.97 (s, 2H), 4.79 (m, 1H), 3.94 (s, 3H), 3.28 (m, 1H), 2.89 (m, 2H), 2.75 (m, 5H), 2.09 (br, 1H), 1.84 (m, 1H), 1.69 (m, 1H), 1.59 (m, 1H), 1.42 (m, 1H), 1.28 (m, 3H), 0.95 (s, 3H)

### Example 86. (S)-quinuclidin-3-yl((R)-5-(3-chloro-4-ethoxyphenyl)-6-fluoro-2,2-dimethyl-2,3-dihydro-1H-inden-1-yl)carbamate

¹H-NMR (400 MHZ, CDCl₃) δ 7.54 (s, 1H), 7.36 (d, 1H), 7.17 (d, 1H), 6.99 (m, 2H), 4.99 (m, 2H), 4.80 (m, 1H), 4.13(q, 2H), 3.27 (m, 1H), 2.89 (m, 2H), 2.77 (m, 5H), 2.08 (m, 1H), 1.85 (m, 1H), 1.70 (m, 1H), 1.59 (m, 1H), 1.50 (t, 3H), 1.42 (m, 1H), 1.29 (s, 3H), 0.96 (s, 3H)

### Example 87. (S)-quinuclidin-3-yl((R)-5-(3-chloro-4-propoxyphenyl)-6-fluoro-2,2-dimethyl-2,3-dihydro-1H-inden-1-yl)carbamate

¹H-NMR (400 MHZ, CDCl₃) δ 7.54 (s, 1H), 7.36 (d, 1H), 7.17 (d, 1H), 6.99 (m, 2H), 4.99 (m, 2H), 4.80 (m, 1H), 4.05 (t, 2H), 3.27 (m, 1H), 2.89 (m, 2H), 2.77 (m, 5H), 2.08 (m, 1H), 1.88 (m, 3H), 1.70 (m, 1H), 1.59 (m, 1H), 1.50 (t, 3H), 1.42 (m, 1H), 1.29 (s, 3H), 1.26 (t, 3H), 0.96 (s, 3H)

### Example 88. (S)-quinuclidin-3-yl((R)-5-(4-butoxy-3-chlorophenyl)-6-fluoro-2,2-dimethyl-2,3-dihydro-1H-inden-1-yl)carbamate

¹H-NMR (400 MHZ, CDCl₃) δ 7.53 (s, 1H), 7.36 (d, 1H), 7.16 (d, 1H), 6.96 (m, 2H), 4.97 (s, 2H), 4.80 (m, 1H), 4.05 (t, 2H), 3.27 (m, 1H), 2.89 (m, 2H), 2.77 (m, 5H), 2.08 (m, 1H), 1.87 (m, 3H), 1.70 (m, 1H), 1.56 (m, 3H), 1.42 (m, 1H), 1.28 (s, 3H), 1.02 (t, 3H), 0.96 (s, 3H)

### Example 89. (S)-quinuclidin-3-yl((R)-6-fluoro-5-(3-fluoro-4-methoxyphenyl)-2,2-dimethyl-2,3-dihydro-1H-inden-1-yl)carbamate

¹H-NMR (400 MHZ, CDCl₃) δ 7.30 (m, 1H), 7.24 (m, 1H), 7.17 (d, 1H), 7.02 (m, 2H), 4.99˜4.89 (m, 2H), 4.80 (m, 1H), 3.94 (s, 3H), 3.29 (m, 1H), 2.89˜2.68 (m, 7H), 2.09 (br, 1H), 1.83 (m, 1H), 1.69 (m, 1H), 1.59 (m, 1H), 1.42 (m, 1H), 1.29 (s, 3H), 0.97 (s, 3H)

### Example 90. (S)-quinuclidin-3-yl((R)-5-(4-ethoxy-3-fluorophenyl)-6-fluoro-2,2-dimethyl-2,3-dihydro-1H-inden-1-yl)carbamate

¹H-NMR (400 MHZ, CDCl₃) δ 7.30 (m, 1H), 7.24 (m, 1H), 7.17 (d, 1H), 7.02 (m, 2H), 4.99˜4.89 (m, 2H), 4.80 (m, 1H), 4.14(q, 2H), 3.29 (m, 1H), 2.89˜2.68 (m, 7H), 2.09 (br, 1H), 1.83 (m, 1H), 1.69 (m, 1H), 1.59 (m, 1H), 1.49 (t, 3H), 1.42 (m, 1H), 1.29 (s, 3H), 0.96 (s, 3H)

### Example 91. (S)-quinuclidin-3-yl((R)-6-fluoro-5-(3-fluoro-4-propoxyphenyl)-2,2-dimethyl-2,3-dihydro-1H-inden-1-yl)carbamate

¹H-NMR (400 MHZ, CDCl₃) δ 7.29 (m, 1H), 7.23 (m, 1H), 7.18 (m, 1H), 7.01 (m, 2H), 4.98˜4.95 (m, 2H), 4.80 (m, 1H), 4.05 (t, 2H), 3.27 (m, 1H), 2.89˜2.67 (m, 7H), 2.09 (br, 1H), 1.84 (m, 3H), 1.69 (m, 1H), 1.59 (m, 1H), 1.42 (m, 1H), 1.29 (s, 3H), 1.09 (t, 3H), 0.96 (s, 3H)

### Example 92. (S)-quinuclidin-3-yl((R)-5-(2-chloro-4-isopropoxyphenyl)-6-fluoro-2,2-dimethyl-2,3-dihydro-1H-inden-1-yl)carbamate

¹H-NMR (400 MHZ, CDCl₃) δ 7.18 (d, 1H), 7.06 (d, 1H), 7.00 (m, 2H), 6.83 (m, 1H), 4.99 (s, 2H), 4.80 (m, 1H), 4.56 (m, 1H), 3.26 (m, 1H), 2.90˜2.67 (m, 7H), 2.09 (m, 1H), 1.83 (m, 1H), 1.68 (m, 1H), 1.58 (m, 1H), 1.42 (m, 1H), 1.37 (d, 6H), 1.29 (s, 3H), 0.98 (s, 3H)

### Example 93. (S)-quinuclidin-3-yl((R)-5-(4-ethyl-3-fluorophenyl)-6-fluoro-2,2-dimethyl-2,3-dihydro-1H-inden-1-yl)carbamate

¹H-NMR (400 MHZ, CDCl₃) δ 7.27 (m, 1H), 7.20 (m, 3H), 7.04 (d, 1H), 4.93 (m, 2H), 4.81 (m, 1H), 3.26 (m, 1H), 2.90˜2.74 (m, 7H), 2.70(q, 2H), 2.09 (m, 1H), 1.84 (m, 1H), 1.69 (m, 1H), 1.59 (m, 1H), 1.42 (m, 1H), 1.29 (s, 3H), 1.27 (t, 3H), 0.97 (s, 3H)

### Example 94. (S)-quinuclidin-3-yl((R)-6-fluoro-5-(4-isopropoxy-3-methylphenyl)-2,2-dimethyl-2,3-dihydro-1H-inden-1-yl)carbamate

¹H-NMR (400 MHZ, CDCl₃) o 7.30 (m, 2H), 7.19 (m, 1H), 7.00 (m, 1H), 6.88 (m, 1H), 4.98˜4.90 (m, 2H), 4.80 (m, 1H), 4.57 (m, 1H), 3.27 (m, 1H), 2.89˜2.67 (m, 7H), 2.26 (s, 3H), 2.09 (m, 1H), 1.85 (m, 1H), 1.69 (m, 1H), 1.59 (m, 1H), 1.42 (m, 1H), 1.38 (d, 6H), 1.29 (s, 3H), 0.97 (s, 3H)

### Example 95. (S)-quinuclidin-3-yl((R)-6-fluoro-5-(4-isopropoxy-3-methoxyphenyl)-2,2-dimethyl-2,3-dihydro-1H-inden-1-yl)carbamate

¹H-NMR (400 MHZ, CDCl₃) δ 7.21 (d, 1H), 7.06 (s, 1H), 7.00 (m, 2H), 6.95 (m, 1H), 4.98˜4.89 (m, 2H), 4.80 (m, 1H), 4.58 (m, 1H), 3.90 (s, 3H), 2.90˜2.69 (m, 7H), 2.09 (br, 1H), 1.85 (m, 1H), 1.69 (m, 1H), 1.59 (m, 1H), 1.42 (m, 1H), 1.40 (d, 6H), 1.29 (s, 3H), 0.98 (s, 3H)

### Example 96. (S)-quinuclidin-3-yl((R)-6-fluoro-5-(4-isopropoxy-2-methylphenyl)-2,2-dimethyl-2,3-dihydro-1H-inden-1-yl)carbamate

¹H-NMR (400 MHZ, CDCl₃) δ 7.10 (d, 1H), 7.00 (m, 2H), 6.80 (m, 1H), 6.75 (m, 1H), 4.96 (m, 2H), 4.81 (m, 1H), 4.57 (m, 1H), 3.28 (m, 1H), 2.89˜2.67 (m, 7H), 2.16 (s, 3H), 2.09 (br, 1H), 1.84 (m, 1H), 1.69 (m, 1H), 1.59 (m, 1H), 1.42 (m, 1H), 1.37 (d, 6H), 1.29 (s, 3H), 0.98(s, 3H Example 97. (S)-quinuclidin-3-yl((R)-5-(3,5-difluoro-4-isopropoxyphenyl)-6-fluoro-2,2-dimethyl-2,3-dihydro-1H-inden-1-yl)carbamate

¹H-NMR (400 MHZ, CDCl₃) δ 7.18 (d, 1H), 7.09 (m, 2H), 7.03 (d, 1H), 5.00˜4.90 (m, 2H), 4.80 (m, 1H), 4.50 (m, 1H), 3.29 (m, 1H), 2.89˜2.68 (m, 7H), 2.09 (br, 1H), 1.84 (m, 1H), 1.69 (m, 1H), 1.59 (m, 1H), 1.42 (m, 1H), 1.37 (d, 6H), 1.29 (s, 3H), 0.96 (s, 3H)

### Example 98. (S)-quinuclidin-3-yl((R)-5-(2-chloro-4-(cyclopropylmethoxy)phenyl)-6-fluoro-2,2-dimethyl-2,3-dihydro-1H-inden-1-yl)carbamate

¹H-NMR (400 MHZ, CDCl₃) δ 7.19 (d, 1H), 7.06 (d, 1H), 7.03 (m, 2H), 6.87 (m, 1H), 4.98 (m, 2H), 4.80 (m, 1H), 3.83 (d, 2H), 3.27 (m, 1H), 2.89˜2.68 (m, 7H), 2.09 (m, 1H), 1.83 (m, 1H), 1.70 (m, 1H), 1.59 (m, 1H), 1.42 (m, 1H), 1.29 (s, 3H), 0.98 (s, 3H), 0.68 (d, 2H), 0.34 (d, 2H)

### Example 99. (S)-quinuclidin-3-yl((R)-5-(3-chloro-4-(cyclopropylmethoxy)phenyl)-6-fluoro-2,2-dimethyl-2,3-dihydro-1H-inden-1-yl)carbamate

¹H-NMR (400 MHZ, CDCl₃) δ 7.54 (s, 1H), 7.36 (d, 1H), 7.17 (d, 1H), 7.20˜6.96 (m, 2H), 4.98˜4.90 (m, 2H), 4.80 (m, 1H), 3.93 (d, 2H), 3.28 (m, 1H), 2.91˜2.68 (m, 7H), 2.09 (br, 1H), 1.84 (m, 1H), 1.69 (m, 1H), 1.59 (m, 1H), 1.42 (m, 1H), 1.29 (s, 3H), 0.97 (s, 3H), 0.68 (d, 2H), 0.37 (d, 2H)

### Example 100. (S)-quinuclidin-3-yl((R)-5-(3,5-dichloro-4-isopropoxyphenyl)-6-fluoro-2,2-dimethyl-2,3-dihydro-1H-inden-1-yl)carbamate

¹H-NMR (400 MHZ, CDCl₃) δ 7.46 (s, 2H), 7.18 (d, 1H), 7.03 (m, 1H), 5.00˜4.91 (m, 2H), 4.80 (m, 1H), 4.66 (m, 1H), 3.28 (m, 1H), 2.89˜2.68 (m, 7H), 2.09 (br, 1H), 1.85 (m, 1H), 1.69 (m, 1H), 1.59 (m, 1H), 1.42 (m, 1H), 1.40 (d, 6H), 1.29 (s, 3H), 0.96 (s, 3H)

### Example 101. (S)-quinuclidin-3-yl((R)-5-(4-(tert-butoxy)phenyl)-6-fluoro-2,2-dimethyl-2,3-dihydro-1H-inden-1-yl)carbamate

¹H-NMR (400 MHz, CDCl₃) δ 7.43 (d, 2H), 7.20 (d, 1H), 7.03 (m, 3H), 4.96 (m, 2H), 4.79 (m, 1H), 3.28 (m, 1H), 2.89˜2.67 (m, 7H), 2.09 (m, 1H), 1.85 (m, 1H), 1.69 (m, 1H), 1.59 (m, 1H), 1.42 (m, 1H), 1.39 (s, 9H), 1.29 (s, 3H), 0.95 (s, 3H)

### Example 102. (S)-quinuclidin-3-yl((R)-5-(4-(tert-butoxy)-2-chlorophenyl)-6-fluoro-2,2-dimethyl-2,3-dihydro-1H-inden-1-yl)carbamate

¹H-NMR (400 MHz, CDCl₃) δ 7.18 (d, 1H), 7.12 (m, 1H), 7.07 (m, 1H), 6.95 (m, 1H), 6.93 (m, 1H), 4.98 (m, 2H), 4.80 (m, 1H), 3.28 (m, 1H), 2.93˜2.68 (m, 7H), 2.09 (m, 1H), 1.85 (m, 1H), 1.69 (m, 1H), 1.59 (m, 1H), 1.42 (m, 1H), 1.40 (s, 9H), 1.29 (s, 3H), 0.98 (s, 3H)

### Example 103. (S)-quinuclidin-3-yl((R)-5-(2-chloro-4-methoxyphenyl)-6-fluoro-2,2-dimethyl-2,3-dihydro-1H-inden-1-yl)carbamate

¹H-NMR (400 MHZ, CDCl₃) δ 7.21 (d, 1H), 7.07 (d, 1H), 7.03 (m, 2H), 6.87 (m, 1H), 4.98 (m, 2H), 4.80 (m, 1H), 3.84 (s, 3H), 3.28 (m, 1H), 2.93˜2.68 (m, 7H), 2.09 (m, 1H), 1.85 (m, 1H), 1.69 (m, 1H), 1.59 (m, 1H), 1.42 (m, 1H), 1.29 (s, 3H), 0.95 (s, 3H)

### Example 104. (S)-quinuclidin-3-yl((R)-5-(2-chloro-4-ethoxyphenyl)-6-fluoro-2,2-dimethyl-2,3-dihydro-1H-inden-1-yl)carbamate

¹H-NMR (400 MHZ, CDCl₃) δ 7.19 (d, 1H), 7.07 (d, 1H), 7.01 (m, 2H), 6.85 (m, 1H), 4.98 (m, 2H), 4.80 (m, 1H), 4.05(q, 2H), 3.28 (m, 1H), 2.89˜2.68 (m, 7H), 2.09 (m, 1H), 1.86 (m, 1H), 1.69 (m, 1H), 1.59 (m, 1H), 1.44 (t, 3H), 1.42 (m, 1H), 1.29 (s, 3H), 0.98 (s, 3H)

### Example 105. (S)-quinuclidin-3-yl((R)-5-(4-ethoxy-3-methylphenyl)-6-fluoro-2,2-dimethyl-2,3-dihydro-1H-inden-1-yl)carbamate

¹H-NMR (400 MHZ, CDCl₃) δ 7.30 (m, 2H), 7.19 (d, 1H), 7.01 (d, 1H), 6.88 (d, 1H), 4.95 (m, 2H), 4.80 (m, 1H), 4.08(q, 2H), 3.27 (m, 1H), 2.93˜2.67 (m, 7H), 2.27 (s, 3H), 2.09 (m, 1H), 1.84 (m, 1H), 1.69 (m, 1H), 1.59 (m, 1H), 1.45 (t, 3H), 1.42 (m, 1H), 1.29 (s, 3H), 0.97 (s, 3H)

### Example 106. (S)-quinuclidin-3-yl((R)-6-fluoro-2,2-dimethyl-5-(3-methyl-4-propoxyphenyl)-2,3-dihydro-1H-inden-1-yl)carbamate

¹H-NMR (400 MHZ, CDCl₃) δ 7.30 (m, 2H), 7.18 (d, 1H), 7.00 (d, 1H), 6.86 (d, 1H), 4.98˜4.88 (m, 2H), 4.80 (m, 1H), 3.98 (t, 2H), 3.27 (m, 1H), 2.89˜2.68 (m, 7H), 2.28 (s, 3H), 2.09 (m, 1H), 1.86 (m, 3H), 1.69 (m, 1H), 1.59 (m, 1H), 1.42 (m, 1H), 1.29 (s, 3H), 1.05 (t, 3H), 0.96 (s, 3H)

### Example 107. (S)-quinuclidin-3-yl((R)-5-(4-(difluoromethoxy)-3-methylphenyl)-6-fluoro-2,2-dimethyl-2,3-dihydro-1H-inden-1-yl)carbamate

¹H-NMR (400 MHZ, CDCl₃) δ 7.38 (s, 1H), 7.32 (d, 1H), 7.17 (d, 1H), 7.13 (d, 1H), 7.02 (d, 1H), 6.73˜6.36 (t, 1H), 4.88˜4.90 (m, 2H), 4.80 (m, 1H), 3.28 (m, 1H), 2.89˜2.68 (m, 7H), 2.34 (s, 3H), 2.09 (br, 1H), 1.84 (m, 1H), 1.69 (m, 1H), 1.59 (m, 1H), 1.42 (m, 1H), 1.29 (s, 3H), 0.95 (s, 3H)

### Example 108. (S)-quinuclidin-3-yl((R)-6-fluoro-5-(3-fluoro-5-isopropoxyphenyl)-2,2-dimethyl-2,3-dihydro-1H-inden-1-yl)carbamate

¹H-NMR (400 MHZ, CDCl₃) δ 7.19 (d, 1H), 7.02 (d, 1H), 6.83 (s, 1H), 6.80 (d, 1H), 6.61 (d, 1H), 4.99˜4.90 (m, 2H), 4.80 (m, 1H), 4.56 (m, 1H), 3.26 (m, 1H), 2.89˜2.68 (m, 7H), 2.09 (br, 1H), 1.85 (m, 1H), 1.69 (m, 1H), 1.59 (m, 1H), 1.42 (m, 1H), 1.37 (d, 6H), 1.29 (s, 3H), 0.97 (s, 3H)

### Example 109. (S)-quinuclidin-3-yl((R)-6-fluoro-5-(3-fluoro-5-isobutoxyphenyl)-2,2-dimethyl-2,3-dihydro-1H-inden-1-yl)carbamate

¹H-NMR (400 MHZ, CDCl₃) δ 7.20 (d, 1H), 7.02 (d, 1H), 6.84 (s, 1H), 6.83 (d, 1H), 6.61 (d, 1H), 4.99˜4.90 (m, 1H), 4.80 (m, 1H), 3.73 (d, 2H), 3.28 (m, 1H), 2.89˜2.68 (m, 7H), 2.09 (br, 2H), 1.85 (m, 1H), 1.69 (m, 1H), 1.59 (m, 1H), 1.42 (m, 1H), 1.29 (s, 3H), 1.03 (d, 6H), 0.97 (s, 3H)

### Example 110. (S)-quinuclidin-3-yl((R)-6-fluoro-5-(3-fluoro-5-(2,2,2-trifluoroethoxy)phenyl)-2,2-dimethyl-2,3-dihydro-1H-inden-1-yl)carbamate

¹H-NMR (400 MHZ, CDCl₃) δ 7.19 (d, 1H), 7.04 (d, 1H), 6.92 (d, 1H), 6.90 (s, 1H), 6.69 (m, 1H), 5.00˜4.90 (m, 2H), 4.80 (m, 1H), 4.30(q, 2H), 3.28 (m, 1H), 2.89˜2.68 (m, 7H), 2.09 (br, 1H), 1.85 (m, 1H), 1.69 (m, 1H), 1.59 (m, 1H), 1.42 (m, 1H), 1.29 (s, 3H), 0.97 (s, 3H)

### Example 111. (S)-quinuclidin-3-yl((R)-5-(4-chloro-3-isopropoxyphenyl)-6-fluoro-2,2-dimethyl-2,3-dihydro-1H-inden-1-yl)carbamate

¹H-NMR (400 MHZ, CDCl₃) δ 7.40 (d, 1H), 7.19 (d, 1H), 7.10 (s, 1H), 7.02 (d, 2H), 5.00˜4.90 (m, 2H), 4.80 (m, 1H), 4.59 (m, 1H), 3.28 (m, 1H), 2.89˜2.68 (m, 7H), 2.09 (br, 1H), 1.85 (m, 1H), 1.69 (m, 1H), 1.59 (m, 1H), 1.42 (m, 1H), 1.40 (d, 6H), 1.29 (s, 3H), 1.02 (d, 6H), 0.97 (s, 3H)

### Example 112. (S)-quinuclidin-3-yl((R)-5-(3-chloro-5-isobutoxyphenyl)-6-fluoro-2,2-dimethyl-2,3-dihydro-1H-inden-1-yl)carbamate

¹H-NMR (400 MHZ, CDCl₃) o 7.19 (d, 1H), 7.07 (s, 1H), 7.01 (d, 1H), 6.94 (s, 1H), 6.88 (s, 1H), 5.00˜4.90 (m, 2H), 4.80 (m, 1H), 3.75 (d, 2H), 3.28 (m, 1H), 2.89˜2.68 (m, 7H), 2.09 (m, 2H), 1.85 (m, 1H), 1.69 (m, 1H), 1.59 (m, 1H), 1.42 (m, 1H), 1.40 (d, 6H), 1.29 (s, 3H), 0.97 (s, 3H)

### Example 113. (S)-quinuclidin-3-yl((R)-5-(3-chloro-5-isopropoxyphenyl)-6-fluoro-2,2-dimethyl-2,3-dihydro-1H-inden-1-yl)carbamate

¹H-NMR (400 MHZ, CDCl₃) δ 7.18 (d, 1H), 7.06 (s, 1H), 7.02 (d, 1H), 6.93 (s, 1H), 6.88 (s, 1H), 4.99˜4.92 (m, 2H), 4.80 (m, 1H), 4.56 (m, 1H), 3.27 (m, 1H), 2.89˜2.67 (m, 7H), 2.09 (br, 1H), 1.84 (m, 1H), 1.69 (m, 1H), 1.59 (m, 1H), 1.42 (m, 1H), 1.38 (d, 6H), 1.29 (s, 3H), 0.97 (s, 3H)

### Example 114. (S)-quinuclidin-3-yl((R)-5-(2-chloro-4-(2,2,2-trifluoroethoxy)phenyl)-6-fluoro-2,2-dimethyl-2,3-dihydro-1H-inden-1-yl)carbamate

¹H-NMR (400 MHZ, CDCl₃) δ 7.38 (s, 1H), 7.34 (d, 1H), 7.20 (m, 1H), 7.07˜7.03 (m, 2H), 5.00 (m, 2H), 4.80 (m, 1H), 3.29 (m, 1H), 2.89˜2.69 (m, 7H), 2.09 (m, 1H), 1.83 (m, 1H), 1.69 (m, 1H), 1.59 (m, 1H), 1.43 (m, 1H), 1.29 (s, 3H), 0.99 (s, 3H)

### Examples 115 to 126

The titled compounds of Examples 115 to 126 were prepared in accordance with the same procedures as in Example 1, using (S)-quinuclidin-3-yl((R)-5-bromo-2,2,6-trimethyl-2,3-dihydro-1H-inden-1-yl)carbamate prepared in Preparation 3; and the corresponding substituted-phenylboronic acids, respectively.

### Example 115. (S)-quinuclidin-3-yl((R)-5-(4-ethylphenyl)-2,2,6-trimethyl-2,3-dihydro-1H-inden-1-yl)carbamate

¹H-NMR (400 MHZ, CDCl₃) δ 7.23 (m, 4H), 7.13 (s, 1H), 7.04 (s, 1H), 4.97˜4.80 (m, 2H), 4.72 (m, 1H), 3.29 (m, 1H), 2.81 (m, 2H), 2.73 (m, 5H), 2.70 (m, 2H), 2.26 (s, 3H), 2.09 (m, 1H), 1.85 (m, 1H), 1.71 (m, 1H), 1.59 (m, 1H), 1.43 (m, 1H), 1.30 (m, 6H), 0.98 (s, 3H)

### Example 116. (S)-quinuclidin-3-yl((R)-2,2,6-trimethyl-5-(4-propylphenyl)-2,3-dihydro-1H-inden-1-yl)carbamate

¹H-NMR (400 MHZ, CDCl₃) δ 7.21 (s, 4H), 7.13 (s, 1H), 7.04 (s, 1H), 4.97˜4.81 (m, 2H), 4.79 (m, 1H), 3.28 (m, 1H), 2.85 (m, 2H), 2.71 (m, 5H), 2.62 (m, 2H), 2.26 (s, 3H), 2.09 (m, 1H), 1.85 (m, 1H), 1.69 (m, 3H), 1.59 (m, 1H), 1.42 (m, 1H), 1.30 (s, 3H), 0.98 (m, 6H)

### Example 117. (S)-quinuclidin-3-yl((R)-5-(4-isopropylphenyl)-2,2,6-trimethyl-2,3-dihydro-1H-inden-1-yl)carbamate

¹H-NMR (400 MHz, CDCl₃) δ 7.25 (m, 4H), 7.13 (s, 1H), 7.04 (s, 1H), 4.94˜4.79 (m, 2H), 4.71 (m, 1H), 3.30 (m, 1H), 2.97 (m, 1H), 2.81 (m, 2H), 2.77 (m, 5H), 2.27 (s, 3H), 2.10 (m, 1H), 1.85 (m, 1H), 1.71 (m, 1H), 1.59 (m, 1H), 1.42 (m, 1H), 1.30 (m, 9H), 0.98 (s, 3H)

### Example 118. (S)-quinuclidin-3-yl((R)-5-(4-butylphenyl)-2,2,6-trimethyl-2,3-dihydro-1H-inden-1-yl)carbamate

¹H-NMR (400 MHZ, CDCl₃) δ 7.21 (s, 4H), 7.13 (s, 1H), 7.04 (s, 1H), 4.96˜4.80 (m, 2H), 4.70 (m, 1H), 3.29 (m, 1H), 2.87 (m, 2H), 2.75 (m, 5H), 2.66 (m, 2H), 2.26 (s, 3H), 2.10 (m, 1H), 1.88˜1.65 (m, 4H), 1.59 (m, 1H), 1.40 (m, 3H), 1.28 (s, 3H), 0.99 (m, 6H)

### Example 119. (S)-quinuclidin-3-yl((R)-5-(4-isobutylphenyl)-2,2,6-trimethyl-2,3-dihydro-1H-inden-1-yl)carbamate

¹H-NMR (400 MHZ, CDCl₃) δ 7.19 (m, 4H), 7.16 (s, 1H), 7.05 (s, 1H), 4.97˜4.79 (m, 2H), 4.68 (m, 1H), 3.29 (m, 1H), 2.89 (m, 2H), 2.77 (m, 5H), 2.52 (d, 2H), 2.26 (s, 3H), 2.10 (m, 1H), 1.91 (m, 1H), 1.87 (m, 1H). 1.71 (m, 1H), 1.59 (m, 1H), 1.42 (m, 1H), 1.30 (s, 3H), 0.98 (s, 3H), 0.94 (d, 6H)

### Example 120. (S)-quinuclidin-3-yl((R)-5-(4-ethoxyphenyl)-2,2,6-trimethyl-2,3-dihydro-1H-inden-1-yl)carbamate

¹H-NMR (400 MHZ, CDCl₃) δ 7.22 (d, 2H), 7.12 (s, 1H), 7.02 (s, 1H), 6.93 (d, 2H), 4.96˜4.79 (m, 2H), 4.70 (m, 1H), 4.07 (m, 2H), 3.28 (m, 1H), 2.81 (m, 2H), 2.72 (m, 5H), 2.25 (s, 3H), 2.09 (m, 1H), 1.86 (m, 1H), 1.70 (m, 1H), 1.59 (m, 1H), 1.46 (t, 3H), 1.43 (m, 1H), 1.28 (s, 3H), 0.98 (s, 3H)

### Example 121. (S)-quinuclidin-3-yl((R)-2,2,6-trimethyl-5-(4-propoxyphenyl)-2,3-dihydro-1H-inden-1-yl)carbamate

¹H-NMR (400 MHZ, CDCl₃) δ 7.21 (d, 2H), 7.12 (s, 1H), 7.02 (s, 1H), 6.93 (d, 2H), 4.96˜4.80 (m, 2H), 4.68 (m, 1H), 3.97 (m, 2H), 3.28 (m, 1H), 2.88 (m, 2H), 2.77 (m, 5H), 2.26 (s, 3H), 2.09 (m, 1H), 1.84 (m, 3H), 1.70 (m, 1H), 1.59 (m, 1H), 1.42 (m, 1H), 1.28 (s, 3H), 1.07 (t, 3H), 0.97 (s, 3H)

### Example 122. (S)-quinuclidin-3-yl((R)-5-(4-isopropoxyphenyl)-2,2,6-trimethyl-2,3-dihydro-1H-inden-1-yl)carbamate

¹H-NMR (400 MHZ, CDCl₃) δ 7.20 (d, 2H), 7.12 (s, 1H), 7.03 (s, 1H), 6.92 (d, 2H), 4.96˜4.81 (m, 2H), 4.79 (m, 1H), 4.60 (m, 1H), 3.29 (m, 1H), 2.88 (m, 2H), 2.74 (m, 5H), 2.26 (s, 3H), 2.09 (m, 1H), 1.85 (m, 1H), 1.69 (m, 1H), 1.59 (m, 1H), 1.38 (m, 1H), 1.27 (d, 6H), 1.29 (s, 3H), 0.97 (s, 3H)

### Example 123. (S)-quinuclidin-3-yl((R)-5-(4-butoxyphenyl)-2,2,6-trimethyl-2,3-dihydro-1H-inden-1-yl)carbamate

¹H-NMR (400 MHZ, CDCl₃) δ 7.21 (d, 2H), 7.12 (s, 1H), 7.02 (s, 1H), 6.93 (d, 2H), 4.96˜4.79 (m, 2H), 4.70 (m, 1H), 3.29 (m, 1H), 2.78˜2.72 (m, 7H), 2.26 (s, 3H), 2.09 (m, 1H), 1.86 (m, 1H), 1.82 (m, 2H), 1.68 (m, 1H), 1.59 (m, 1H), 1.51 (m, 2H), 1.42 (m, 1H), 1.28 (s, 3H), 1.01 (m, 6H)

### Example 124. (S)-quinuclidin-3-yl((R)-5-(4-isobutoxyphenyl)-2,2,6-trimethyl-2,3-dihydro-1H-inden-1-yl)carbamate

¹H-NMR (400 MHZ, CDCl₃) δ 7.21 (d, 2H), 7.12 (s, 1H), 7.02 (s, 1H), 6.93 (d, 2H), 4.96˜4.80 (m, 2H), 4.71 (m, 1H), 3.76 (d, 2H), 3.30 (m, 1H), 2.85 (m, 2H), 2.75 (m, 5H), 2.26 (s, 3H), 2.10 (m, 2H), 1.85 (m, 1H), 1.69 (m, 1H), 1.60 (m, 1H), 1.42 (m, 1H), 1.30 (s, 3H), 1.05 (d, 6H), 0.98 (s, 3H)

### Example 125. (S)-quinuclidin-3-yl((R)-5-(3-chloro-4-isopropoxyphenyl)-2,2,6-trimethyl-2,3-dihydro-1H-inden-1-yl)carbamate

¹H-NMR (400 MHZ, CDCl₃) δ 7.31 (s, 1H), 7.11 (m, 2H), 7.00˜6.96 (m, 2H), 4.96˜4.80 (m, 2H), 4.71 (m, 1H), 4.60 (m, 1H), 3.30 (m, 1H), 2.85 (m, 2H), 2.77 (m, 5H), 2.25 (s, 3H), 2.09 (m, 1H), 1.85 (m, 1H), 1.70 (m, 1H), 1.60 (m, 1H), 1.42 (m, 1H), 1.41 (d, 6H), 1.29 (s, 3H), 0.98 (s, 3H)

### Example 126. (S)-quinuclidin-3-yl((R)-5-(2-fluoro-4-methoxyphenyl)-2,2,6-trimethyl-2,3-dihydro-1H-inden-1-yl)carbamate

¹H-NMR (400 MHZ, CDCl₃) δ 7.13 (m, 2H), 7.00 (s, 1H), 6.76˜6.69 (m, 2H), 4.97˜4.79 (m, 2H), 4.69 (m, 1H), 3.85 (s, 3H), 3.28 (m, 1H), 2.88 (m, 2H), 2.77 (m, 5H), 2.18 (s, 3H), 2.09 (m, 1H), 1.85 (m, 1H), 1.71 (m, 1H), 1.59 (m, 1H), 1.42 (m, 1H), 1.28 (s, 3H), 0.97 (s, 3H)

### EXAMPLES 127 TO 138

The titled compounds of Examples 127 to 138 were prepared in accordance with the same procedures as in Example 1, using (S)-quinuclidin-3-yl((R)-5-bromo-6-ethoxy-2,2-dimethyl-2,3-dihydro-1H-inden-1-yl)carbamate prepared in Preparation 4; and the corresponding substituted-phenylboronic acids, respectively.

### Example 127. (S)-quinuclidin-3-yl((R)-6-ethoxy-5-(4-ethylphenyl)-2,2-dimethyl-2,3-dihydro-1H-inden-1-yl)carbamate

¹H-NMR (400 MHZ, CDCl₃) δ 7.46 (d, 2H), 7.22 (d, 2H), 7.12 (s, 1H), 6.85 (m, 1H), 4.96˜4.80 (m, 2H), 4.73 (m, 1H), 4.00 (m, 2H), 3.28 (m, 1H), 2.91˜2.78 (m, 7H), 2.69 (m, 2H), 2.10 (m, 1H), 1.87 (m, 1H), 1.72 (m, 1H), 1.60 (m, 1H), 1.41 (m, 1H), 1.35 (t, 3H), 1.31 (m, 6H), 0.98 (s, 3H)

### Example 128. (S)-quinuclidin-3-yl((R)-6-ethoxy-2,2-dimethyl-5-(4-propylphenyl)-2,3-dihydro-1H-inden-1-yl)carbamate

¹H-NMR (400 MHZ, CDCl₃) δ 7.45 (d, 2H), 7.21 (d, 2H), 7.12 (s, 1H), 6.87 (m, 1H), 4.96˜4.80 (m, 2H), 4.73 (m, 1H), 4.00 (m, 2H), 3.28 (m, 1H), 2.91˜2.69 (m, 7H), 2.60 (m, 2H), 2.10 (m, 1H), 1.87 (m, 1H), 1.69 (m, 3H), 1.59 (m, 1H), 1.41 (m, 1H), 1.33 (t, 3H), 1.30 (s, 3H), 0.99 (m, 6H)

### Example 129. (S)-quinuclidin-3-yl((R)-6-ethoxy-5-(4-isopropylphenyl)-2,2-dimethyl-2,3-dihydro-1H-inden-1-yl)carbamate

¹H-NMR (400 MHZ, CDCl₃) δ 7.47 (d, 2H), 7.26 (m, 2H), 7.13 (s, 1H), 6.85 (m, 1H), 4.96˜4.80 (m, 2H), 4.73 (m, 1H), 4.01 (m, 2H), 3.25 (m, 1H), 2.91 (m, 1H), 2.90 (m, 2H), 2.77 (m, 5H), 2.10 (m, 1H), 1.86 (m, 1H), 1.70 (m, 1H), 1.60 (m, 1H), 1.41 (m, 1H), 1.37 (t, 3H), 1.30 (s, 3H), 1.28 (d, 6H), 1.00 (s, 3H)

### Example 130. (S)-quinuclidin-3-yl((R)-5-(4-butylphenyl)-6-ethoxy-2,2-dimethyl-2,3-dihydro-1H-inden-1-yl)carbamate

¹H-NMR (400 MHZ, CDCl₃) δ 7.45 (d, 2H), 7.20 (d, 2H), 7.12 (s, 1H), 6.85 (m, 1H), 4.96˜4.81 (m, 2H), 4.72˜4.70 (m, 1H), 4.00 (m, 2H), 3.30 (m, 1H), 2.90˜2.70 (m, 7H), 2.65 (m, 2H), 2.10 (m, 1H), 1.86 (m, 1H), 1.69 (m, 1H), 1.66 (m, 3H), 1.39 (m, 3H), 1.35 (t, 3H), 1.30 (s, 3H), 0.98 (s, 3H), 0.92 (t, 3H)

### Example 131. (S)-quinuclidin-3-yl((R)-6-ethoxy-5-(4-isobutylphenyl)-2,2-dimethyl-2,3-dihydro-1H-inden-1-yl)carbamate

¹H-NMR (400 MHZ, CDCl₃) δ 7.45 (d, 2H), 7.16 (d, 2H), 7.13 (s, 1H), 6.85 (m, 1H), 4.96˜4.91 (m, 2H), 4.80 (m, 1H), 4.00 (m, 2H), 3.29 (m, 1H), 2.81˜2.70 (m, 7H), 2.50 (d, 2H), 2.10 (m, 1H), 1.91 (m, 1H), 1.85 (m, 1H), 1.71 (m, 1H), 1.59 (m, 1H), 1.41 (m, 1H), 1.34 (t, 3H), 1.29 (s, 3H), 0.97(s 3H), 0.91 (d, 6H)

### Example 132. (S)-quinuclidin-3-yl((R)-6-ethoxy-5-(4-ethoxyphenyl)-2,2-dimethyl-2,3-dihydro-1H-inden-1-yl)carbamate

¹H-NMR (400 MHZ, CDCl₃) δ 7.46 (d, 2H), 7.10 (s, 1H), 6.92 (d, 2H), 6.83 (m, 1H), 4.95˜4.80 (m, 2H), 4.72 (m, 1H), 4.09 (m, 2H), 4.00 (m, 2H), 3.29 (m, 1H), 2.90˜2.70 (m, 7H), 2.10 (m, 1H), 1.86 (m, 1H), 1.70 (m, 1H), 1.59 (m, 1H), 1.46 (t, 3H), 1.44 (m, 1H), 1.34 (t, 3H), 1.30 (s, 3H), 0.98 (s, 3H)

### Example 133. (S)-quinuclidin-3-yl((R)-6-ethoxy-2,2-dimethyl-5-(4-propoxyphenyl)-2,3-dihydro-1H-inden-1-yl) carbamate

¹H-NMR (400 MHZ, CDCl₃) δ 7.46 (d, 2H), 7.10 (s, 1H), 6.92 (d, 2H), 6.83 (m, 1H), 4.95˜4.80 (m, 2H), 4.71 (m, 1H), 3.98 (m, 2H), 3.95 (m, 2H), 3.30 (m, 1H), 2.90˜2.72 (m, 7H), 2.10 (m, 1H), 1.88 (m, 1H), 1.85 (m, 2H), 1.70 (m, 1H), 1.59 (m, 1H), 1.43 (m, 1H), 1.34 (t, 3H), 1.30 (s, 3H), 1.05 (t, 3H), 0.98 (s, 3H)

### Example 134. (S)-quinuclidin-3-yl((R)-6-ethoxy-5-(4-isopropoxyphenyl)-2,2-dimethyl-2,3-dihydro-1H-inden-1-yl)carbamate

¹H-NMR (400 MHZ, CDCl₃) δ 7.44 (d, 2H), 7.09 (s, 1H), 6.89 (d, 2H), 6.81 (m, 1H), 4.92 (s, 2H), 4.77 (m, 1H), 4.56 (m, 1H), 4.00 (m, 2H), 3.29 (m, 1H), 2.89˜2.68 (m, 7H), 2.10 (m, 1H), 1.85 (m, 1H), 1.70 (m, 1H), 1.59 (m, 1H), 1.42 (m, 1H), 1.32 (d, 6H), 1.29 (t, 3H), 1.25 (s, 3H), 0.98 (s, 3H)

### Example 135. (S)-quinuclidin-3-yl((R)-5-(4-butoxyphenyl)-6-ethoxy-2,2-dimethyl-2,3-dihydro-1H-inden-1-yl)carbamate

¹H-NMR (400 MHZ, CDCl₃) δ 7.45 (d, 2H), 7.09 (s, 1H), 6.90 (d, 2H), 6.83 (m, 1H), 4.90 (m, 2H), 4.78 (m, 1H), 4.00 (m, 4H), 3.27 (m, 1H), 2.89˜2.69 (m, 7H), 2.10 (m, 1H), 1.85 (m, 1H), 1.79 (m, 2H), 1.70 (m, 1H), 1.59 (m, 1H), 1.51 (m, 2H), 1.42 (m, 1H), 1.33 (t, 3H), 1.27 (s, 3H), 0.99 (m, 6H)

### Example 136. (S)-quinuclidin-3-yl((R)-6-ethoxy-5-(4-isobutoxyphenyl)-2,2-dimethyl-2,3-dihydro-1H-inden-1-yl)carbamate

¹H-NMR (400 MHZ, CDCl₃) δ 7.44 (d, 2H), 7.09 (s, 1H), 6.91 (d, 2H), 6.84 (m, 1H), 4.90 (m, 2H), 4.79 (m, 1H), 4.00 (m, 2H), 3.75 (d, 2H), 3.27 (m, 1H), 2.88˜2.69 (m, 7H), 2.08 (m, 2H), 1.85 (m, 1H), 1.71 (m, 1H), 1.58 (m, 1H), 1.42 (m, 1H), 1.33 (t, 3H), 1.27 (s, 3H), 1.04 (d, 6H), 0.99 (s, 3H)

### Example 137. (S)-quinuclidin-3-yl((R)-5-(3-chloro-4-isopropoxyphenyl)-6-ethoxy-2,2-dimethyl-2,3-dihydro-1H-inden-1-yl)carbamate

¹H-NMR (400 MHz, CDCl₃) δ 7.57 (s, 1H), 7.38 (d, 1H), 7.09 (s, 1H), 6.96 (d, 1H), 6.82 (m, 1H), 4.95˜4.85 (m, 2H), 4.75 (m, 1H), 4.60 (m, 1H), 4.00 (m, 2H), 3.28 (m, 1H), 2.90˜2.68 (m, 7H), 2.09 (m, 1H), 1.84 (m, 1H), 1.70 (m, 1H), 1.59 (m, 1H), 1.42 (m, 1H), 1.41 (d, 6H), 1.35 (t, 3H), 1.27 (s, 3H), 0.99 (s, 3H)

### Example 138. (S)-quinuclidin-3-yl((R)-6-ethoxy-5-(2-fluoro-4-methoxyphenyl)-2,2-dimethyl-2,3-dihydro-1H-inden-1-yl)carbamate

¹H-NMR (400 MHZ, CDCl₃) δ 7.22 (m, 1H), 7.04 (s, 1H), 6.85 (m, 1H), 6.74˜6.67 (m, 2H), 4.96˜4.87 (m, 2H), 4.80 (m, 1H), 4.00 (m, 2H), 3.84 (s, 3H), 3.29 (m, 1H), 2.90˜2.70 (m, 7H), 2.10 (m, 1H), 1.83 (m, 1H), 1.70 (m, 1H), 1.60 (m, 1H), 1.43 (m, 1H), 1.31˜1.27 (m, 6H), 0.99 (s, 3H)

### EXAMPLES 139 TO 169

The titled compounds of Examples 139 to 169 were prepared in accordance with the same procedures as in Example 1, using (S)-quinuclidin-3-yl((R)-6-bromo-2,2-dimethyl-1,2,3,4-tetrahydronaphthalen-1-yl)carbamate prepared in Preparation 5; and the corresponding substituted-phenylboronic acids, respectively.

### Example 139. (S)-quinuclidin-3-yl((R)-6-(4-ethylphenyl)-2,2-dimethyl-1,2,3,4-tetrahydronaphthalen-1-yl)carbamate

¹H-NMR (400 MHZ, CDCl₃) δ 7.50 (d, 2H), 7.40 (m, 1H), 7.34 (m, 2H), 7.27 (m, 2H), 4.82 (m, 2H), 4.64 (d, 1H), 3.29 (m, 1H), 2.90˜2.71 (m, 7H), 2.68 (m, 2H), 2.10 (m, 1H), 1.84 (m, 1H), 1.74 (m, 3H), 1.59 (m, 1H), 1.43 (m, 1H), 1.29 (t, 3H), 1.06 (s, 3H), 0.95 (s, 3H)

### Example 140. (S)-quinuclidin-3-yl((R)-2,2-dimethyl-6-(4-propylphenyl)-1,2,3,4-tetrahydronaphthalen-1-yl)carbamate

¹H-NMR (400 MHZ, CDCl₃) δ 7.50 (d, 2H), 7.41 (m, 1H), 7.34 (m, 2H), 7.25 (m, 2H), 4.81 (m, 2H), 4.64 (d, 1H), 3.30 (m, 1H), 2.90˜2.74 (m, 7H), 2.63 (m, 2H), 2.10 (m, 1H), 1.85 (m, 1H), 1.70 (m, 3H), 1.58 (m, 1H), 1.42 (m, 1H), 1.06 (s, 3H), 0.95 (m, 6H)

### Example 141. (S)-quinuclidin-3-yl((R)-6-(4-isopropylphenyl)-2,2-dimethyl-1,2,3,4-tetrahydronaphthalen-1-yl)carbamate

¹H-NMR (400 MHZ, CDCl₃) δ 7.50 (d, 2H), 7.40 (m, 1H), 7.36˜7.27 (m, 4H), 4.82 (m, 2H), 4.67 (m, 1H), 3.30 (m, 1H), 2.97 (m, 1H), 2.89˜2.78 (m, 7H), 2.10 (m, 1H), 1.85 (m, 1H), 1.71 (m, 3H), 1.59 (m, 1H), 1.42 (m, 1H), 1.29 (d, 6H), 1.06 (s, 3H), 0.95 (s, 3H)

### Example 142. (S)-quinuclidin-3-yl((R)-6-(4-butylphenyl)-2,2-dimethyl-1,2,3,4-tetrahydronaphthalen-1-yl) carbamate

¹H-NMR (400 MHZ, CDCl₃) o 7.49 (d, 2H), 7.40 (m, 1H), 7.34 (m, 2H), 7.26 (m, 2H), 4.81 (m, 2H), 4.65 (m, 1H), 3.28 (m, 1H), 2.90˜2.78 (m, 7H), 2.65 (m, 2H), 2.10 (m, 1H), 1.85 (m, 1H), 1.71 (m, 3H), 1.68 (m, 2H), 1.62 (m, 1H), 1.38 (m, 3H), 1.06 (s, 3H), 0.95 (m, 6H)

### Example 143. (S)-quinuclidin-3-yl((R)-6-(4-ethoxyphenyl)-2,2-dimethyl-1,2,3,4-tetrahydronaphthalen-1-yl) carbamate

¹H-NMR (400 MHZ, CDCl₃) δ 7.50 (d, 2H), 7.35 (m, 2H), 7.27 (m, 1H), 6.96 (d, 2H), 4.83 (m, 2H), 4.66 (d, 1H), 4.14 (m, 2H), 3.30 (m, 1H), 2.90˜2.74 (m, 7H), 2.10 (m, 1H), 1.85 (m, 1H), 1.71 (m, 3H), 1.59 (m, 1H), 1.46 (t, 3H), 1.42 (m, 1H), 1.06 (s, 3H), 0.95 (s, 3H)

### Example 144. (S)-quinuclidin-3-yl((R)-2,2-dimethyl-6-(4-propoxyphenyl)-1,2,3,4-tetrahydronaphthalen-1-yl) carbamate

¹H-NMR (400 MHZ, CDCl₃) δ 7.50 (d, 2H), 7.36 (m, 1H), 7.32 (m, 1H), 7.27 (m, 1H), 6.96 (d, 2H), 4.80 (m, 2H), 4.66 (d, 1H), 3.96 (m, 2H), 3.30 (m, 1H), 2.89˜2.78 (m, 7H), 2.10 (m, 1H), 1.84 (m, 3H), 1.70 (m, 3H), 1.59 (m, 1H), 1.40 (m, 1H), 1.06 (m, 6H), 0.94 (s, 3H)

### Example 145. (S)-quinuclidin-3-yl((R)-6-(4-butoxyphenyl)-2,2-dimethyl-1,2,3,4-tetrahydronaphthalen-1-yl)carbamate

¹H-NMR (400 MHZ, CDCl₃) δ 7.50 (d, 1H), 7.37 (m, 1H), 7.32 (m, 1H), 7.28 (m, 1H), 6.96 (d, 2H), 4.80 (m, 2H), 4.63 (d, 1H), 4.00 (m, 2H), 3.30 (m, 1H), 2.90˜2.73 (m, 7H), 2.10 (m, 1H), 1.83 (m, 1H), 1.81 (m, 2H), 1.71 (m, 3H), 1.59 (m, 1H), 1.51 (m, 2H), 1.42 (m, 1H), 1.06 (s, 3H), 0.95 (m, 6H)

### Example 146. (S)-quinuclidin-3-yl((R)-6-(4-isobutoxyphenyl)-2,2-dimethyl-1,2,3,4-tetrahydronaphthalen-1-yl)carbamate

¹H-NMR (400 MHz, CDCl₃) δ 7.50 (d, 2H), 7.37 (m, 1H), 7.32 (m, 1H), 7.29 (m, 1H), 6.96 (d, 2H), 4.81 (m, 2H), 4.66 (m, 1H), 3.76 (d, 2H), 3.30 (m, 1H), 2.89˜2.77 (m, 7H), 2.09 (m, 2H), 1.83 (m, 1H), 1.70 (m, 3H), 1.59 (m, 1H), 1.42 (m, 1H), 1.06 (d, 9H), 0.95 (s, 3H)

### Example 147. (S)-quinuclidin-3-yl((R)-6-(4-methoxy-3,5-dimethylphenyl)-2,2-dimethyl-1,2,3,4-tetrahydronaphthalen-1-yl) carbamate

¹H-NMR (400 MHZ, CDCl₃) δ 7.34 (m, 2H), 7.27 (m, 1H), 7.22 (s, 2H), 4.81 (m, 2H), 4.66 (m, 1H), 3.77 (s, 3H), 3.30 (m, 1H), 2.88˜2.74 (m, 7H), 2.35 (s, 6H), 2.10 (m, 1H), 1.84 (m, 1H), 1.70 (m, 3H), 1.59 (m, 1H), 1.41 (m, 1H), 1.05 (s, 3H), 0.97 (s, 3H)

### Example 148. (S)-quinuclidin-3-yl((R)-6-(3,5-dimethyl-4-propoxyphenyl)-2,2-dimethyl-1,2,3,4-tetrahydronaphthalen-1-yl)carbamate

¹H-NMR (400 MHZ, CDCl₃) δ 7.36 (m, 2H), 7.27 (m, 1H), 7.21 (s, 2H), 4.83 (m, 2H), 4.66 (d, 1H), 3.78 (m, 2H), 3.30 (m, 1H). 2.90˜2.74 (m, 7H), 2.33 (s, 6H), 2.10 (m, 1H), 1.87 (m, 3H), 1.71 (m, 3H), 1.59 (m, 1H), 1.41 (m, 1H), 1.10 (t, 3H), 1.06 (s, 3H), 0.97 (s, 3H)

### Example 149. (S)-quinuclidin-3-yl((R)-6-(4-isopropoxy-3,5-dimethylphenyl)-2,2-dimethyl-1,2,3,4-tetrahydronaphthalen-1-yl) carbamate

¹H-NMR (400 MHZ, CDCl₃) δ 7.37 (m, 1H), 7.30 (m, 2H), 7.21 (s, 2H), 4.81 (m, 2H), 4.66 (d, 1H), 4.21 (m, 1H), 3.30 (m, 1H), 2.89˜2.74 (m, 7H), 2.33 (s, 6H), 2.10 (m, 1H), 1.84 (m, 1H), 1.70 (m, 3H), 1.59 (m, 1H), 1.42 (m, 1H), 1.31 (d, 6H), 1.06 (s, 3H), 0.94(s, 3H)

### Example 150. (S)-quinuclidin-3-yl((R)-6-(3-chloro-4-isopropoxyphenyl)-2,2-dimethyl-1,2,3,4-tetrahydronaphthalen-1-yl) carbamate

¹H-NMR (400 MHZ, CDCl₃) δ 7.59 (s, 1H), 7.40 (d, 1H), 7.35 (s, 2H), 7.29 (m, 1H), 7.00 (d, 1H), 4.80 (m, 2H), 4.64 (m, 1H), 4.59 (m, 1H), 3.29 (m, 1H), 2.89˜2.74 (m, 7H), 2.09 (m, 1H), 1.84 (m, 1H), 1.70 (m, 3H), 1.59 (m, 2H), 1.41 (d, 6H), 1.05 (s, 3H), 0.95 (s, 3H)

### Example 151. (S)-quinuclidin-3-yl((R)-6-(2-fluoro-4-methoxyphenyl)-2,2-dimethyl-1,2,3,4-tetrahydronaphthalen-1-yl) carbamate

¹H-NMR (400 MHZ, CDCl₃) δ 7.34 (m, 3H), 7.26 (m, 1H), 6.78˜6.69 (m, 2H), 4.83 (m, 2H), 4.66 (m, 1H), 3.84 (s, 3H), 3.30 (m, 1H), 2.89˜2.73 (m, 7H), 2.09 (m, 1H), 1.84 (m, 1H), 1.71 (m, 3H), 1.60 (m, 1H), 1.42 (m, 1H), 1.06 (s, 3H), 0.95 (s, 3H)

### Example 152. (S)-quinuclidin-3-yl((R)-6-(4-butoxy-3,5-dimethylphenyl)-2,2-dimethyl-1,2,3,4-tetrahydronaphthalen-1-yl)carbamate

¹H-NMR (400 MHZ, CDCl₃) δ 7.36 (m, 1H), 7.31 (m, 2H), 7.21 (s, 2H), 4.81 (m, 2H), 4.63 (m, 1H), 3.80 (m, 2H), 3.28 (m, 1H), 2.87˜2.78 (m, 7H), 2.34 (s, 6H), 2.08 (m, 1H), 1.82 (m, 3H), 1.71 (m, 3H), 1.58 (m, 3H), 1.42 (m, 1H), 1.05 (s, 3H), 1.01 (t, 3H), 0.97 (s, 3H)

### Example 153. (S)-quinuclidin-3-yl((R)-6-(3-methoxyphenyl)-2,2-dimethyl-1,2,3,4-tetrahydronaphthalen-1-yl)carbamate

¹H-NMR (400 MHZ, CDCl₃) δ 7.42 (m, 1H), 7.35 (m, 3H), 7.17 (d, 1H), 7.11 (s, 1H), 6.90 (m, 1H), 4.85 (m, 2H), 4.67 (m, 1H), 3.87 (s, 3H), 3.30 (m, 1H), 2.90˜2.75 (m, 7H), 2.09 (m, 1H), 1.83 (m, 1H), 1.72 (m, 3H), 1.59 (m, 1H), 1.42 (m, 1H), 1.06 (s, 3H), 0.95 (s, 3H)

### Example 154. (S)-quinuclidin-3-yl((R)-6-(3-ethoxyphenyl)-2,2-dimethyl-1,2,3,4-tetrahydronaphthalen-1-yl)carbamate

¹H-NMR (400 MHZ, CDCl₃) δ 7.40 (m, 1H), 7.34 (m, 3H), 7.15 (d, 1H), 7.11 (s, 1H), 6.87 (m, 1H), 4.83 (m, 2H), 4.65 (m, 1H), 4.10 (m, 2H), 3.30 (m, 1H), 2.89˜2.76 (m, 7H), 2.10 (m, 1H), 1.84 (m, 1H), 1.70 (m, 3H), 1.45 (t, 3H), 1.42 (m, 1H), 1.06 (s, 3H), 0.97 (s, 3H)

### Example 155. (S)-quinuclidin-3-yl((R)-6-(3-butoxyphenyl)-2,2-dimethyl-1,2,3,4-tetrahydronaphthalen-1-yl)carbamate

¹H-NMR (400 MHZ, CDCl₃) δ 7.43 (m, 1H), 7.35 (m, 3H), 7.14 (d, 1H), 7.11 (s, 1H), 6.88 (m, 1H), 3.81 (m, 2H), 4.68 (m, 1H), 4.03 (m, 2H), 3.29 (m, 1H), 2.89˜2.74 (m, 7H), 2.10 (m, 1H), 1.82 (m, 3H), 1.71 (m, 3H), 1.60 (m, 1H), 1.51 (m, 2H), 1.42 (m, 1H), 1.06 (s, 3H), 0.99 (m, 6H)

### Example 156. (S)-quinuclidin-3-yl((R)-2,2-dimethyl-6-(3-propylphenyl)-1,2,3,4-tetrahydronaphthalen-1-yl) carbamate

¹H-NMR (400 MHZ, CDCl₃) δ 7.41˜7.32 (m, 6H), 7.16 (m, 1H), 4.81 (m, 2H), 4.65 (m, 1H), 3.30 (m, 1H), 2.89˜2.68 (m, 7H), 2.64 (m, 2H), 2.10 (m, 1H), 1.84 (m, 1H), 1.71 (m, 3H), 1.60 (m, 1H), 1.42 (m, 1H), 1.06 (s, 3H), 0.96 (m, 6H)

### Example 157. (S)-quinuclidin-3-yl((R)-6-(3-isobutylphenyl)-2,2-dimethyl-1,2,3,4-tetrahydronaphthalen-1-yl)carbamate

¹H-NMR (400 MHZ, CDCl₃) δ 7.42˜7.34 (m, 6H), 7.13 (d, 1H), 4.81 (m, 2H), 4.62 (d, 1H), 3.30 (m, 1H), 2.91˜2.74 (m, 7H), 2.54 (d, 2H), 2.10 (m, 1H), 1.92 (m, 1H), 1.84 (m, 1H), 1.71 (m, 3H), 1.59 (m, 1H), 1.41 (m, 1H), 1.06 (s, 3H), 0.95 (s, 3H), 0.92 (d, 6H)

### Example 158. (S)-quinuclidin-3-yl((R)-6-(3-ethylphenyl)-2,2-dimethyl-1,2,3,4-tetrahydronaphthalen-1-yl)carbamate

¹H-NMR (400 MHZ, CDCl₃) δ 7.42˜7.33 (m, 6H), 7.19 (d, 1H), 4.81 (m, 2H), 4.66 (d, 1H), 3.30 (m, 1H), 2.89˜2.74 (m, 7H), 2.70 (m, 2H), 2.10 (m, 1H), 1.85 (m, 1H), 1.71 (m, 3H), 1.59 (m, 1H), 1.42 (m, 1H), 1.27 (t, 3H), 1.06 (s, 3H), 0.95 (s, 3H)

### Example 159. (S)-quinuclidin-3-yl((R)-2,2-dimethyl-6-(3-propoxyphenyl)-1,2,3,4-tetrahydronaphthalen-1-yl)carbamate

¹H-NMR (400 MHZ, CDCl₃) δ 7.41 (m, 1H), 7.36˜7.32 (m, 3H), 7.14 (d, 1H), 7.10 (s, 1H), 6.87 (m, 1H), 4.82 (m, 2H), 4.65 (m, 1H), 3.98 (m, 2H), 3.30 (m, 1H), 2.90˜2.74 (m, 7H), 2.10 (m, 1H), 1.86(, 3H), 1.71 (m, 3H), 1.59 (m, 1H), 1.42 (m, 1H), 1.06 (s, 3H), 1.04 (t, 3H), 0.98 (s, 3H)

### Example 160. (S)-quinuclidin-3-yl((R)-6-(4-isobutylphenyl)-2,2-dimethyl-1,2,3,4-tetrahydronaphthalen-1-yl)carbamate

¹H-NMR (400 MHZ, CDCl₃) δ 7.48 (d, 2H), 7.41 (m, 1H), 7.34 (m, 2H), 7.21 (d, 2H), 4.81 (m, 2H), 4.64 (m, 1H), 3.30 (m, 1H), 2.90˜2.74 (m, 7H), 2.51 (d, 2H), 2.10 (m, 1H), 1.94 (m, 1H), 1.88 (m, 1H), 1.71 (m, 3H), 1.59 (m, 1H), 1.42 (m, 1H), 1.06 (s, 3H), 0.98 (s, 3H), 0.91 (d, 6H)

### Example 161. (S)-quinuclidin-3-yl((R)-6-(3-fluoro-4-isopropoxyphenyl)-2,2-dimethyl-1,2,3,4-tetrahydronaphthalen-1-yl)carbamate

¹H-NMR (400 MHZ, CDCl₃) δ 7.35 (s, 2H), 7.32 (m, 1H), 7.27 (m, 2H), 7.03 (t, 1H), 4.81 (m, 2H), 4.64 (m, 1H), 4.56 (m, 1H), 3.30 (m, 1H), 2.89˜2.74 (m, 7H), 2.10 (m, 1H), 1.85 (m, 1H), 1.71 (m, 3H), 1.60 (m, 1H), 1.41 (m, 1H), 1.40 (d, 6H), 1.06 (s, 3H), 0.95 (s, 3H)

### Example 162. (S)-quinuclidin-3-yl((R)-6-(3-isopropylphenyl)-2,2-dimethyl-1,2,3,4-tetrahydronaphthalen-1-yl)carbamate

¹H-NMR (400 MHZ, CDCl₃) δ 7.42˜7.33 (m, 6H), 7.22 (d, 1H), 4.81 (m, 2H), 4.66 (m, 1H), 3.30 (m, 1H), 2.98 (m, 1H), 2.96˜2.74 (m, 7H), 2.10 (m, 1H), 1.85 (m, 1H), 1.71 (m, 3H), 1.59 (m, 1H), 1.42 (m, 1H), 1.29 (d, 6H), 1.06 (s, 3H), 0.95 (s, 3H)

### Example 163. (S)-quinuclidin-3-yl((R)-6-(3-isobutoxyphenyl)-2,2-dimethyl-1,2,3,4-tetrahydronaphthalen-1-yl)carbamate

¹H-NMR (400 MHZ, CDCl₃) δ 7.41 (m, 1H), 7.35 (m, 3H), 7.14 (d, 1H), 7.11 (s, 1H), 6.88 (m, 1H), 4.82 (m, 2H), 4.67 (m, 1H), 3.79 (d, 2H), 3.30 (m, 1H), 2.90˜2.74 (m, 7H), 2.10 (m, 2H), 1.84 (m, 1H), 1.71 (m, 3H), 1.59 (m, 1H), 1.42 (m, 1H), 1.05 (d, 9H), 0.95 (s, 3H)

### Example 164. (S)-quinuclidin-3-yl((R)-6-(4-ethoxy-3,5-dimethylphenyl)-2,2-dimethyl-1,2,3,4-tetrahydronaphthalen-1-yl)carbamate

¹H-NMR (400 MHZ, CDCl₃) δ 7.36˜7.31 (m, 2H), 7.27 (m, 1H), 7.21 (s, 2H), 4.81 (m, 2H), 4.66 (m, 1H), 3.89 (m, 2H), 3.28 (m, 1H), 2.89˜2.74 (m, 7H), 2.34 (s, 6H), 2.09 (m, 1H), 1.84 (m, 1H), 1.70 (m, 3H), 1.59 (m, 1H), 1.45 (t, 3H), 1.41 (m, 1H), 1.05 (s, 3H), 0.95 (s, 3H)

### Example 165. (S)-quinuclidin-3-yl((R)-6-(3-isopropoxyphenyl)-2,2-dimethyl-1,2,3,4-tetrahydronaphthalen-1-yl)carbamate

¹H-NMR (400 MHZ, CDCl₃) δ 7.40 (m, 1H), 7.34 (s, 1H), 7.31 (m, 2H), 7.14 (d, 1H), 7.10 (s, 1H), 6.87 (d, 1H), 4.80 (m, 2H), 4.67 (m, 1H), 4.61 (m, 1H), 3.29 (m, 1H), 2.89˜2.74 (m, 7H), 2.08 (m, 1H), 1.84 (m, 1H), 1.72 (m, 3H), 1.59 (m, 1H), 1.41 (m, 1H), 1.37 (d, 6H), 1.06 (s, 3H), 0.95 (s, 3H)

### Example 166. (S)-quinuclidin-3-yl((R)-6-(4-methoxyphenyl)-2,2-dimethyl-1,2,3,4-tetrahydronaphthalen-1-yl)carbamate

¹H-NMR (400 MHZ, CDCl₃) δ 7.51 (d, 2H), 7.33 (m, 2H), 7.28 (m, 1H), 6.98 (d, 2H), 4.83 (m, 2H), 4.65 (m, 1H), 3.86 (s, 3H), 3.28 (m, 1H), 2.89˜2.77 (m, 7H), 2.08 (m, 1H), 1.80 (m, 1H), 1.70(m 3H), 1.59 (m, 1H), 1.41 (m, 1H), 1.06 (s, 3H), 0.95 (s, 3H)

### Example 167. (S)-quinuclidin-3-yl((R)-6-(4-isopropoxyphenyl)-2,2-dimethyl-1,2,3,4-tetrahydronaphthalen-1-yl)carbamate

¹H-NMR (400 MHZ, CDCl₃) δ 7.50 (d, 2H), 7.37˜7.32 (m, 2H), 7.29 (m, 1H), 6.95 (d, 2H), 4.81 (m, 2H), 4.62 (m, 1H), 4.59 (m, 1H), 3.29 (m, 1H), 2.89˜2.74 (m, 7H), 2.09 (m, 1H), 1.83 (m, 1H), 1.73 (m, 3H), 1.58 (m, 1H), 1.41 (m, 1H), 1.38 (d, 6H), 1.06 (s, 3H), 0.95 (s, 3H)

### Example 168. (S)-quinuclidin-3-yl((R)-6-(2-chloro-4-isopropoxyphenyl)-2,2-dimethyl-1,2,3,4-tetrahydronaphthalen-1-yl)carbamate

¹H-NMR (400 MHZ, CDCl₃) δ 7.32 (m, 1H), 7.25 (m, 2H), 7.15 (s, 1H), 7.00 (s, 1H), 6.82 (d, 1H), 4.90 (m, 1H), 4.80 (m, 1H), 4.64 (m, 1H), 4.55 (m, 1H), 3.25 (m, 1H), 2.93˜2.73 (m, 7H), 2.08 (m, 1H), 1.83 (m, 2H), 1.70 (m, 3H), 1.59 (m, 1H), 1.40 (m, 1H), 1.38 (d, 6H), 1.06 (s, 3H), 0.97 (s, 3H)

### Example 169. (S)-quinuclidin-3-yl((R)-6-(4-ethyl-3-fluorophenyl)-2,2-dimethyl-1,2,3,4-tetrahydronaphthalen-1-yl)carbamate

¹H-NMR (400 MHZ, CDCl₃) δ 7.36 (m, 2H), 7.27˜7.21 (m, 4H), 4.86 (d, 1H), 4.79 (m, 1H), 4.64 (m, 1H), 3.26 (m, 1H), 2.93˜2.71 (m, 7H), 2.69(q, 2H), 2.09 (m, 1H), 1.83 (m, 2H), 1.81 (m, 1H), 1.70 (m, 3H), 1.59 (m, 1H), 1.40 (m, 1H), 1.27 (t, 3H), 1.06 (s, 3H), 0.97 (s, 3H)

### Examples 170 to 199

The titled compounds of Examples 170 to 199 were prepared in accordance with the same procedures as in Example 1, using (S)-quinuclidin-3-yl((R)-6-bromo-7-fluoro-2,2-dimethyl-1,2,3,4-tetrahydronaphthalen-1-yl)carbamate prepared in Preparation 6; and the corresponding substituted-phenylboronic acids, respectively.

### Example 170. (S)-quinuclidin-3-yl((R)-6-(4-ethylphenyl)-7-fluoro-2,2-dimethyl-1,2,3,4-tetrahydronaphthalen-1-yl)carbamate

¹H-NMR (400 MHZ, CDCl₃) δ 7.46 (d, 2H), 7.28 (d, 2H), 7.15 (d, 1H), 7.08 (d, 1H), 4.84 (m, 2H), 4.65 (d, 1H), 3.28 (m, 1H), 2.81˜2.71 (m, 7H), 2.69 (m, 2H), 2.10 (m, 1H), 1.83 (m, 1H), 1.70 (m, 3H), 1.59 (m, 1H), 1.42 (m, 1H), 1.30 (t, 3H), 1.06 (s, 3H), 0.95 (s, 3H)

### Example 171. (S)-quinuclidin-3-yl((R)-7-fluoro-2,2-dimethyl-6-(4-propylphenyl)-1,2,3,4-tetrahydronaphthalen-1-yl)carbamate

¹H-NMR (400 MHZ, CDCl₃) δ 7.45 (d, 2H), 7.24 (m, 2H), 7.15 (d, 1H), 7.07 (d, 1H), 4.81 (m, 2H), 4.64 (m, 1H), 3.28 (m, 1H), 2.89˜2.76 (m, 7H), 2.63 (m, 2H), 2.09 (m, 1H), 1.84 (m, 1H), 1.68 (m, 3H), 1.59 (m, 1H), 1.42 (m, 1H), 1.05 (s, 3H), 1.00 (t, 3H), 0.95 (s, 3H)

### Example 172. (S)-quinuclidin-3-yl((R)-7-fluoro-6-(4-isopropylphenyl)-2,2-dimethyl-1,2,3,4-tetrahydronaphthalen-1-yl) carbamate

¹H-NMR (400 MHZ, CDCl₃) δ 7.46 (d, 2H), 7.30 (d, 2H), 7.15 (d, 1H), 7.08 (d, 1H), 4.81 (m, 2H), 4.63 (m, 1H), 3.29 (m, 1H), 2.98 (m, 1H), 2.90˜2.77 (m, 7H), 2.10 (m, 1H), 1.85 (m, 1H), 1.73 (m, 3H), 1.63 (m, 1H), 1.41 (m, 1H), 1.39 (d, 6H), 1.05 (s, 3H), 0.95 (s, 3H)

### Example 173. (S)-quinuclidin-3-yl((R)-6-(4-butylphenyl)-7-fluoro-2,2-dimethyl-1,2,3,4-tetrahydronaphthalen-1-yl)carbamate

¹H-NMR (400 MHZ, CDCl₃) δ 7.45 (d, 2H), 7.26 (d, 2H), 7.15 (d, 1H), 7.07 (d, 1H), 4.82 (m, 2H), 4.63 (m, 1H), 3.29 (m, 1H), 2.89˜2.76 (m, 7H), 2.66 (m, 2H), 2.10 (m, 1H), 1.83 (m, 1H), 1.71 (m, 3H), 1.66 (m, 2H), 1.59 (m, 1H), 1.38 (m, 3H), 1.06 (s, 3H), 0.95 (s, 3H)

### Example 174. (S)-quinuclidin-3-yl((R)-7-fluoro-6-(4-isobutylphenyl)-2,2-dimethyl-1,2,3,4-tetrahydronaphthalen-1-yl)carbamate

¹H-NMR (400 MHZ, CDCl₃) δ 7.44 (d, 2H), 7.22 (d, 2H), 7.17 (d, 1H), 7.07 (d, 1H), 4.82 (m, 2H), 4.61 (m, 1H), 3.29 (m, 1H), 2.89˜2.76 (m, 7H), 2.52 (d, 2H), 2.10 (m, 1H), 1.93 (m, 1H), 1.89 (m, 1H), 1.70 (m, 3H), 1.60 (m, 1H), 1.42 (m, 1H), 1.05 (s, 3H), 0.95 (m, 9H)

### Example 175. (S)-quinuclidin-3-yl((R)-7-fluoro-6-(4-methoxyphenyl)-2,2-dimethyl-1,2,3,4-tetrahydronaphthalen-1-yl) carbamate

¹H-NMR (400 MHZ, CDCl₃) δ 7.47 (d, 2H), 7.13 (d, 1H), 7.08 (d, 1H), 6.98 (d, 2H), 4.87 (m, 1H), 4.79 (m, 1H), 4.62 (m, 1H), 3.86 (s, 3H), 3.30 (m, 1H), 2.89˜2.76 (m, 7H), 2.10 (m, 1H), 1.83 (m, 1H), 1.71 (m, 3H), 1.60 (m, 1H), 1.42 (m, 1H), 1.06 (s, 3H), 0.95 (s, 3H)

### Example 176. (S)-quinuclidin-3-yl((R)-6-(4-ethoxyphenyl)-7-fluoro-2,2-dimethyl-1,2,3,4-tetrahydronaphthalen-1-yl)carbamate

¹H-NMR (400 MHZ, CDCl₃) δ 7.46 (d, 2H), 7.13 (d, 1H), 7.07 (d, 1H), 6.96 (d, 2H), 4.85˜4.79 (m, 2H), 4.63 (m, 1H), 4.08 (m, 2H), 3.28 (m, 1H), 2.89˜2.76 (m, 7H), 2.09 (m, 1H), 1.85 (m, 1H), 1.71 (m, 3H), 1.59 (m, 1H), 1.46 (t, 3H), 1.41 (m, 1H), 1.06 (s, 3H), 0.95 (s, 3H)

### Example 177. (S)-quinuclidin-3-yl((R)-7-fluoro-2,2-dimethyl-6-(4-propoxyphenyl)-1,2,3,4-tetrahydronaphthalen-1-yl) carbamate

¹H-NMR (400 MHZ, CDCl₃) δ 7.46 (d, 2H), 7.12 (d, 1H), 7.07 (d, 1H), 6.96 (d, 2H), 4.81 (m, 2H), 4.60 (m, 1H), 3.97 (m, 2H), 3.30 (m, 1H), 2.89˜2.76 (m, 7H), 2.10 (m, 1H), 1.85 (m, 3H), 1.71 (m, 3H), 1.59 (m, 1H), 1.42 (m, 1H), 1.06 (s, 3H), 1.04 (t, 3H), 0.95 (s, 3H)

### Example 178. (S)-quinuclidin-3-yl((R)-6-(4-butoxyphenyl)-7-fluoro-2,2-dimethyl-1,2,3,4-tetrahydronaphthalen-1-yl)carbamate

¹H-NMR (400 MHZ, CDCl₃) δ 7.45 (d, 2H), 7.12 (d, 1H), 7.05 (d, 1H), 6.96 (d, 2H), 4.87 (m, 2H), 4.67 (m, 1H), 4.00 (m, 2H), 3.28 (m, 1H), 2.89˜2.79 (m, 7H), 2.09 (m, 1H), 1.81 (m, 3H), 1.70 (m, 3H), 1.59 (m, 1H), 1.50 (m, 2H), 1.41 (m, 1H), 1.06 (s, 3H), 0.99 (t, 3H), 0.94 (s, 3H)

### Example 179. (S)-quinuclidin-3-yl((R)-7-fluoro-6-(4-isobutoxyphenyl)-2,2-dimethyl-1,2,3,4-tetrahydronaphthalen-1-yl)carbamate

¹H-NMR (400 MHZ, CDCl₃) δ 7.45 (d, 2H), 7.12 (d, 1H), 7.07 (d, 1H), 6.96 (d, 2H), 4.82 (m, 2H), 4.64 (m, 1H), 3.76 (d, 2H), 3.28 (m, 1H), 2.89˜2.76 (m, 7H), 2.11 (m, 1H), 2.10 (m, 1H), 1.83 (m, 1H), 1.71 (m, 3H), 1.60 (m, 1H), 1.42 (m, 1H), 1.06 (d, 9H), 0.95 (s, 3H)

### Example 180. (S)-quinuclidin-3-yl((R)-7-fluoro-6-(4-methoxy-3,5-dimethylphenyl)-2,2-dimethyl-1,2,3,4-tetrahydronaphthalen-1-yl)carbamate

¹H-NMR (400 MHZ, CDCl₃) δ 7.18 (s, 2H), 7.11 (s, 1H), 7.06 (d, 1H), 4.83 (m, 2H), 4.64 (m, 1H), 3.77 (s, 3H), 3.29 (m, 1H), 2.89˜2.79 (m, 7H), 2.34 (s, 6H), 2.10 (m, 1H), 1.84 (m, 1H), 1.71 (m, 3H), 1.60 (m, 1H), 1.42 (m, 1H), 1.06 (s, 3H), 0.95 (s, 3H)

### Example 181. (S)-quinuclidin-3-yl((R)-6-(3,5-dimethyl-4-propoxyphenyl)-7-fluoro-2,2-dimethyl-1,2,3,4-tetrahydronaphthalen-1-yl)carbamate

¹H-NMR (400 MHZ, CDCl₃) δ 7.17 (s, 2H), 7.12 (d, 1H), 7.07 (d, 1H), 4.83 (m, 2H), 4.62 (m, 1H), 3.78 (m, 2H), 3.30 (m, 1H), 2.93˜2.79 (m, 7H), 2.33 (s, 6H), 2.10 (m, 1H), 1.84 (m, 3H), 1.71 (m, 3H), 1.60 (m, 1H), 1.42 (m, 1H), 1.10 (t, 3H), 1.06 (s, 3H), 0.95 (s, 3H)

### Example 182. (S)-quinuclidin-3-yl((R)-7-fluoro-6-(4-isopropoxy-3,5-dimethylphenyl)-2,2-dimethyl-1,2,3,4-tetrahydronaphthalen-1-yl)carbamate

¹H-NMR (400 MHZ, CDCl₃) δ 7.17 (s, 2H), 7.13 (d, 1H), 7.06 (d, 1H), 4.84 (m, 2H), 4.62 (m, 1H), 4.22 (m, 1H), 3.28 (m, 1H), 2.89˜2.75 (m, 7H), 2.32 (s, 6H), 2.09 (m, 1H), 1.84 (m, 1H), 1.71 (m, 3H), 1.60 (m, 1H), 1.42 (m, 1H), 1.34 (d, 6H), 1.06 (s, 3H), 0.95 (s, 3H)

### Example 183. (S)-quinuclidin-3-yl((R)-6-(4-butoxy-3,5-dimethylphenyl)-7-fluoro-2,2-dimethyl-1,2,3,4-tetrahydronaphthalen-1-yl)carbamate

¹H-NMR (400 MHz, CDCl₃) δ 7.17 (s, 2H), 7.11 (d, 1H), 7.06 (d, 1H), 4.83 (m, 2H), 4.62 (m, 1H), 3.82 (m, 2H), 3.28 (m, 1H), 2.89˜2.75 (m, 7H), 2.33 (s, 6H), 2.10 (m, 1H), 1.83 (m, 3H), 1.71 (m, 3H), 1.60 (m, 3H), 1.42 (m, 1H), 1.06 (s, 3H), 1.01 (t, 3H), 0.95 (s, 3H)

### Example 184. (S)-quinuclidin-3-yl((R)-7-fluoro-6-(3-methoxyphenyl)-2,2-dimethyl-1,2,3,4-tetrahydronaphthalen-1-yl)carbamate

¹H-NMR (400 MHZ, CDCl₃) δ 7.37 (m, 1H), 7.16 (d, 1H), 7.12 (d, 1H), 7.10 (s, 2H), 6.91 (d, 1H), 4.84 (m, 2H), 4.65 (d, 1H), 3.86 (s, 3H), 3.28 (m, 1H), 2.89˜2.76 (m, 7H), 2.10 (m, 1H), 1.85 (m, 1H), 1.72 (m, 3H), 1.60 (m, 1H), 1.42 (m, 1H), 1.06 (s, 3H), 0.95 (s, 3H)

### Example 185. (S)-quinuclidin-3-yl((R)-6-(3-ethoxyphenyl)-7-fluoro-2,2-dimethyl-1,2,3,4-tetrahydronaphthalen-1-yl)carbamate

¹H-NMR (400 MHZ, CDCl₃) δ 7.34 (m, 1H), 7.16 (d, 1H), 7.11˜7.06 (m, 3H), 6.91 (d, 1H), 4.85 (m, 2H), 4.64 (d, 1H), 4.07 (m, 2H), 3.29 (m, 1H), 2.89˜2.75 (m, 7H), 2.10 (m, 1H), 1.85 (m, 1H), 1.71 (m, 3H), 1.59 (m, 1H), 1.45 (m, 1H), 1.41 (t, 3H), 1.06 (s, 3H), 0.95 (s, 3H)

### Example 186. (S)-quinuclidin-3-yl((R)-6-(3-butoxyphenyl)-7-fluoro-2,2-dimethyl-1,2,3,4-tetrahydronaphthalen-1-yl)carbamate

¹H-NMR (400 MHZ, CDCl₃) δ 7.34 (t, 1H), 7.17 (d, 1H), 7.11˜7.07 (m, 3H), 6.90 (m, 1H), 4.83 (m, 2H), 4.66 (m, 1H), 4.00 (m, 2H), 3.29 (m, 1H), 2.89˜2.76 (m, 7H), 2.10 (m, 1H), 1.82 (m, 1H), 1.79 (m, 2H), 1.71 (m, 3H), 1.60 (m, 1H), 1.52 (m, 2H), 1.42 (m, 1H), 1.06 (s, 3H), 1.00 (t, 3H), 0.95 (s, 3H)

### Example 187. (S)-quinuclidin-3-yl((R)-6-(3-ethylphenyl)-7-fluoro-2,2-dimethyl-1,2,3,4-tetrahydronaphthalen-1-yl)carbamate

¹H-NMR (400 MHZ, CDCl₃) δ 7.35 (m, 3H), 7.21 (m, 1H), 7.16 (d, 1H), 7.09 (d, 1H), 4.84 (m, 2H), 4.66 (m, 1H), 3.29 (m, 1H), 2.89˜2.76 (m, 7H), 2.72 (m, 2H), 2.10 (m, 1H), 1.84 (m, 1H), 1.71 (m, 3H), 1.59(m, 1h), 1.42 (m, 1H), 1.29 (t, 3H), 1.06 (s, 3H), 0.95 (s, 3H)

### Example 188. (S)-quinuclidin-3-yl((R)-7-fluoro-2,2-dimethyl-6-(3-propylphenyl)-1,2,3,4-tetrahydronaphthalen-1-yl)carbamate

¹H-NMR (400 MHZ, CDCl₃) δ 7.35 (m, 3H), 7.15 (m, 2H), 7.07 (d, 1H), 4.85 (m, 1H), 4.79 (m, 1H), 4.63 (d, 1H), 3.29 (m, 1H), 2.89˜2.79 (m, 7H), 2.65 (m, 2H), 2.10 (m, 1H), 1.84 (m, 1H), 1.72 (m, 3H), 1.59 (m, 1H), 1.42 (m, 1H), 1.06 (s, 3H), 0.95 (m, 6H)

### Example 189. (S)-quinuclidin-3-yl((R)-7-fluoro-6-(3-isobutylphenyl)-2,2-dimethyl-1,2,3,4-tetrahydronaphthalen-1-yl)carbamate

¹H-NMR (400 MHZ, CDCl₃) δ 7.34 (m, 2H), 7.31 (s, 1H), 7.17 (m, 2H), 7.07 (d, 1H), 4.87 (d, 1H), 4.80 (m, 1H), 4.66 (d, 1H), 3.30 (m, 1H), 2.89˜2.76 (m, 7H), 2.53 (d, 2H), 2.10 (m, 1H), 1.90 (m, 1H), 1.84 (m, 1H), 1.71 (m, 3H), 1.59 (m, 1H), 1.42 (m, 1H), 1.06 (s, 3H), 0.95 (m, 9H)

### Example 190. (S)-quinuclidin-3-yl((R)-7-fluoro-6-(3-isopropylphenyl)-2,2-dimethyl-1,2,3,4-tetrahydronaphthalen-1-yl)carbamate

¹H-NMR (400 MHZ, CDCl₃) δ 7.38 (m, 3H), 7.25 (m, 1H), 7.16 (d, 1H), 7.09 (d, 1H), 4.83 (m, 2H), 4.63 (d, 1H), 3.30 (m, 1H), 2.98 (m, 1H), 2.89˜2.76 (m, 7H), 2.10 (m, 1H), 1.85 (m, 1H), 1.72 (m, 3H), 1.60 (m, 1H), 1.41 (m, 1H), 1.28 (d, 6H), 1.06 (s, 3H), 0.95 (s, 3H)

### Example 191. (S)-quinuclidin-3-yl((R)-7-fluoro-6-(3-isobutoxyphenyl)-2,2-dimethyl-1,2,3,4-tetrahydronaphthalen-1-yl) carbamate

¹H-NMR (400 MHZ, CDCl₃) δ 7.34 (t, 1H), 7.16 (d, 1H), 7.10 (m, 3H), 6.90 (m, 1H), 4.83 (m, 2H), 4.65 (m, 1H), 3.77 (d, 2H), 3.29 (m, 1H), 2.89˜2.80 (m, 7H), 2.11 (m, 2H), 1.83 (m, 1H), 1.71(m 3H), 1.59 (m, 1H), 1.41 (m, 1H), 1.05 (m, 9H), 0.95 (s, 3H)

### Example 192. (S)-quinuclidin-3-yl((R)-7-fluoro-2,2-dimethyl-6-(3-propoxyphenyl)-1,2,3,4-tetrahydronaphthalen-1-yl) carbamate

¹H-NMR (400 MHZ, CDCl₃) δ 7.36 (t, 1H), 7.18 (d, 1H), 7.10 (m, 3H), 6.90 (m, 1H), 4.83 (m, 2H), 4.65 (d, 1H), 3.97 (m, 2H), 3.30 (m, 1H), 2.89˜2.71 (m, 7H), 2.10 (m, 1H), 1.84 (m, 3H), 1.71 (m, 3H), 1.59 (m, 1H), 1.41 (m, 1H), 1.06 (m, 6H), 0.95 (s, 3H)

### Example 193. (S)-quinuclidin-3-yl((R)-6-(3-chloro-4-isopropoxyphenyl)-7-fluoro-2,2-dimethyl-1,2,3,4-tetrahydronaphthalen-1-yl)carbamate

¹H-NMR (400 MHZ, CDCl₃) δ 7.55 (s, 1H), 7.37 (m, 1H), 7.11 (m, 2H), 6.99 (d, 1H), 4.82 (m, 2H), 4.65 (m, 1H), 4.60 (m, 1H), 3.30 (m, 1H), 2.89˜2.76 (m, 7H), 2.09 (m, 1H), 1.83 (m, 1H), 1.71 (m, 3H), 1.60 (m, 1H), 1.42 (m, 1H), 1.41 (d, 6H), 1.06 (s, 3H), 0.94 (s, 3H)

### Example 194. (S)-quinuclidin-3-yl((R)-7-fluoro-6-(2-fluoro-4-methoxyphenyl)-2,2-dimethyl-1,2,3,4-tetrahydronaphthalen-1-yl)carbamate

¹H-NMR (400 MHZ, CDCl₃) δ 7.29 (m, 1H), 7.08 (d, 2H), 6.75 (m, 2H), 4.84 (m, 2H), 4.66 (m, 1H), 3.85 (s, 3H), 3.30 (m, 1H), 2.86˜2.75 (m, 7H), 2.09 (m, 1H), 1.87 (m, 1H), 1.71 (m, 3H), 1.59 (m, 1H), 1.42 (m, 1H), 1.06 (s, 3H), 0.94 (s, 3H)

### Example 195. (S)-quinuclidin-3-yl((R)-6-(4-ethoxy-3,5-dimethylphenyl)-7-fluoro-2,2-dimethyl-1,2,3,4-tetrahydronaphthalen-1-yl)carbamate

¹H-NMR (400 MHZ, CDCl₃) δ 7.17 (s, 2H), 7.12 (d, 1H), 7.05 (m, 1H), 4.81 (m, 2H), 4.62 (m, 1H), 3.89 (m, 2H), 3.30 (m, 1H), 2.89˜2.79 (m, 7H), 2.33 (s, 6H), 2.09 (m, 1H), 1.82 (m, 1H), 1.73 (m, 3H), 1.60 (m, 1H), 1.46 (t, 3H), 1.41 (m, 1H), 1.06 (s, 3H), 0.94 (s, 3H)

### Example 196. (S)-quinuclidin-3-yl((R)-7-fluoro-6-(3-isopropoxyphenyl)-2,2-dimethyl-1,2,3,4-tetrahydronaphthalen-1-yl)carbamate

¹H-NMR (400 MHZ, CDCl₃) δ 7.33 (t, 1H), 7.10 (d, 1H), 7.10 (s, 1H), 7.07 (m, 2H), 6.91 (d, 1H), 4.83 (m, 2H), 4.66 (m, 1H), 4.60 (m, 1H), 3.29 (m, 1H), 2.89˜2.76 (m, 7H), 2.09 (m, 1H), 1.77 (m, 1H), 1.70 (m, 3H), 1.59 (m, 1H), 1.41 (m, 1H), 1.38 (d, 6H), 1.06 (s, 3H), 0.95 (s, 3H)

### Example 197. (S)-quinuclidin-3-yl((R)-7-fluoro-6-(4-isopropoxyphenyl)-2,2-dimethyl-1,2,3,4-tetrahydronaphthalen-1-yl)carbamate

¹H-NMR (400 MHZ, CDCl₃) δ 7.45 (d, 2H), 7.14 (d, 1H), 7.06 (d, 1H), 6.94 (d, 2H), 4.84 (m, 2H), 4.64 (m, 1H), 4.59 (m, 1H), 3.29 (m, 1H), 2.89˜2.75 (m, 7H), 2.09 (m, 1H), 1.84 (m, 1H), 1.71 (m, 3H), 1.60 (m, 1H), 1.41 (m, 1H), 1.38 (d, 6H), 1.06 (s, 3H), 0.95 (s, 3H)

### Example 198. (S)-quinuclidin-3-yl((R)-7-fluoro-6-(3-fluoro-4-isopropoxyphenyl)-2,2-dimethyl-1,2,3,4-tetrahydronaphthalen-1-yl)carbamate

¹H-NMR (400 MHZ, CDCl₃) δ 7.30˜7.22 (m, 2H), 7.13 (d, 1H), 7.09˜7.00 (m, 2H), 4.84 (m, 2H), 4.65 (m, 1H), 4.60 (m, 1H), 3.30 (m, 1H), 2.90˜2.78 (m, 7H), 2.11 (m, 1H), 1.85 (m, 1H), 1.71 (m, 3H), 1.61 (m, 1H), 1.42 (m, 1H), 1.39 (d, 6H), 1.06 (s, 3H), 0.94 (s, 3H)

### Example 199. (S)-quinuclidin-3-yl((R)-6-(2-chloro-4-isopropoxyphenyl)-7-fluoro-2,2-dimethyl-1,2,3,4-tetrahydronaphthalen-1-yl) carbamate

¹H-NMR (400 MHZ, CDCl₃) δ 7.20 (m, 1H), 7.08 (m, 1H), 7.00 (m, 2H), 6.82 (d, 1H), 4.90 (m, 1H), 4.80 (m, 1H), 4.64 (m, 1H), 4.55 (m, 1H), 3.25 (m, 1H), 2.93˜2.73 (m, 7H), 2.08 (m, 1H), 1.83 (m, 2H), 1.70 (m, 3H), 1.59 (m, 1H), 1.40 (m, 1H), 1.36 (d, 6H), 1.06 (s, 3H), 0.97 (s, 3H)

Experimental Example 1: Evaluation of inhibitory activities against GCS

The inhibitory activities of the compounds of the present invention against GCS were evaluated, as follows, according to the method described in the known literature (Hayashi Y et al., A sensitive and reproducible assay to measure the activity of glucosylceramide synthase and lactosylceramide synthase using HPLC and fluorescent substrates, Analytical Biochemistry 345 (2005) 181-186). Ibiglustat, known as a GCS inhibitor, was used as a control.

### (1) Materials

A549 cells (ATCC, CCL-185)
NBD C₆-ceramide (Thermo Fisher, N1154)
UDP-glucose (Sigma, U4625)
Potassium chloride (Sigma, P9333)
UltraPure™ 0.5 M EDTA (Invitrogen, 15575-038)
BCA protein assay kit (Thermo Fisher, 23227)
Ibiglustat (Shanghai Systeam Biochem Co., ltd, Genz-682452)
HEPES (sigma, H3375)
Protease/phosphatase inhibitor cocktail (CST, 5872s)
DMEM (GIBCO, 11995-065)
FBS (GIBCO, 16000-044)
Antibiotic-Antimycotic (100×) (GIBCO, 15240-112)
200 mM L-glutamine (GIBCO, 25030081)
PBS (GIBCO, 10010-023)
0.25% Trypsin-EDTA (GIBCO, 25200-056)
Dimethyl sulfoxide (Sigma, 34869)
2-Propanol, HPLC grade (Burdick & Jackson, AH323-4)
Hexane, HPLC grade (Burdick & Jackson, AH216-4)
Chloroform (Sigma, C2432)
Methanol (Merck, 1.06009.1011)
(2) Protocol
   <1> Preparation of cell lysates

A549 cells (ATCC, CCL-185) were cultured in a DMEM medium supplemented with 10% fetal bovine serum (FBS), 1× antibiotic-antimycotic, and 1× L-glutamine, in an incubator at 37° C. and 5% CO₂. After the cells attached to the culture dish were washed with phosphate buffered saline (PBS), the cells were scraped off with a cell scraper and then centrifuged (4000 rpm, 3 min, 4ºC) to collect the cells in a 50 ml tube. The cell pellets were suspended in a lysis buffer (50 mM HEPES, pH 7.3, containing 1× the protease/phosphatase inhibitor cocktail), lysed by sonication, and then the lysate was centrifuged (13000 rpm, 10 min, 4° C.). The obtained supernatant was used for the quantitative analysis of proteins. The amount of proteins was measured using the BCA protein assay kit, using bovine serum albumin as a standard.

### <2>GCS Enzyme Reaction

Enzymatic reactions were initiated by sequentially adding the following reaction materials to a 96 deep-well plate. Thereafter, the enzymatic reactions were performed at 37° C. for 90 minutes.

Enzyme reaction mix (Total 50 ul)

| | | | | Volume |
|---|---|---|---|---|
| | Stock Con. | Final Con. | | (ul) |
| 1 | | | | |
| Cpd. | Cpd. 500~0.003 uM (DMSO) | 10000~ | | 1 |
| | | 0.056 nM | | |
| Mix | Total | | | 1 |
| 2 | | | | |
| Sub | 20 mM UDP-Glc | 500 | uM | 1.25 |
| Mix | 25 mM EDTA | 1 | mM | 2 |
| | 500 uM NBD-Cer/BSA | 5 | uM | 0.5 |
| | D.W | | | 35.25 |
| | Total | | | 39 |
| 3 | | | | |

| | | | |
|---|---|---|---|
| Enzyme | 3.135 ug/ul A549 cell lysate | 0.627 ug/ | 10 |
| | | ul(31.35 ug) | |
| Mix | Total | | 10 |

### <3>Lipid Extraction

The enzymatic reactions were stopped by adding 100 μl of chloroform/methanol (2:1, v/v). After vortexing for 15 seconds, each mixture was centrifuged (4000 rpm, 10 min, 18° C.). The lower layer (50 μl) was transferred to a new 96 deep-well plate and dried with a reduced concentrator.

### <4>HPLC Analysis

Lipids were dissolved in 100 μl of isopropyl alcohol/n-hexane/H₂O (55:44:1, v/v/v) and then transferred to a glass vial for HPLC (Agilent, 8004-HP-H/i3u). The sample (100 μl) was automatically loaded onto a normal-phase column (Intersil SIL 150A-5, 4.6×250 mm, GL Sciences, Japan) and then eluted at a flow rate of 2.0 ml/min with isopropyl alcohol/n-hexane/H₂O (55:44:1, v/v/v). The fluorescence thereof was measured with a fluorescence detector (Agilent, 1260 FLD Spectra), using 470 nm and 530 nm as excitation and emission wavelengths, respectively.

### <5>Data Analysis

Data analysis was performed by the following equations.

% Area (Sample)=Area (GlcCer)/[Area (Cer)+Area (GlcCer)]×100%

GCS activity=[Area (Sample)−Area (ibiglustat)]/[Area (DMSO)−Area (ibiglustat)]×100

The obtained % GCS activity data was analyzed with the software GraphPad Prism (Ver 5.01) to calculate the IC₅₀ values. The results are shown in Tables 1 to 3 below.

TABLE 1

| Example | IC₅₀ (nM) |
|---|---|
| 1 | 0.32 |
| 2 | 0.96 |
| 3 | 0.29 |
| 4 | 0.51 |
| 5 | 0.67 |
| 6 | 0.75 |
| 7 | 0.52 |
| 8 | 0.2 |
| 9 | 3.5 |
| 10 | 4.88 |
| 11 | 3.31 |
| 12 | 0.69 |
| 13 | 0.06 |
| 14 | 3.96 |
| 15 | 3.13 |
| 16 | 2.83 |
| 17 | 2.63 |
| 18 | 3.19 |
| 19 | 2.1 |
| 20 | 0.36 |
| 21 | 1.34 |
| 22 | 4.1 |
| 23 | 3.96 |
| 24 | 3.13 |
| 25 | 2.83 |
| 26 | 2.63 |
| 27 | 3.19 |
| 28 | 2.1 |
| 29 | 0.36 |
| 30 | 1.34 |
| 31 | 4.1 |
| 32 | 0.192 |
| 33 | 0.97 |
| 34 | 1.31 |
| 35 | 2.22 |
| 36 | 0.13 |
| 37 | 1.94 |
| 38 | 0.05 |
| 39 | 1.03 |
| 40 | 3.21 |
| 41 | 0.9 |
| 42 | 0.3 |
| 43 | 0.42 |
| 44 | 0.17 |
| 45 | 0.61 |
| 46 | 2.19 |
| 47 | 1.89 |
| 48 | 1.3 |
| 49 | 0.58 |
| 50 | 0.44 |
| 51 | 0.65 |
| 52 | 8.27 |
| 53 | 0.67 |
| 54 | 0.59 |
| 55 | 0.63 |
| 56 | 0.27 |
| 57 | 0.08 |
| 58 | 0.25 |
| 59 | 0.28 |
| 60 | 0.13 |
| 61 | 0.44 |
| 62 | 0.3 |
| 63 | 2.49 |
| 64 | 3.92 |
| 65 | 5.65 |
| 66 | 1.31 |
| 67 | 0.52 |
| 68 | 0.14 |
| 69 | 0.15 |
| 70 | 0.12 |
| 71 | 0.47 |
| 72 | 1.19 |
| 73 | 1.07 |
| 74 | 1.71 |
| 75 | 0.31 |
| 76 | 0.64 |
| 77 | 0.31 |
| 78 | 0.7 |
| 79 | 0.34 |
| 80 | 3.51 |
| 81 | 1.1 |
| 82 | 0.54 |
| 83 | 0.58 |
| 84 | 0.29 |
| 85 | 0.818 |
| 86 | 0.756 |
| 87 | 0.77 |
| 88 | 0.779 |
| 89 | 0.621 |
| 90 | 0.525 |

TABLE 2

| Example | IC₅₀ (nM) |
|---|---|
| 91 | 0.329 |
| 92 | 0.07 |
| 93 | 0.37 |
| 94 | 0.52 |
| 95 | 1.14 |
| 96 | 0.08 |
| 97 | 1.06 |
| 98 | 0.06 |
| 99 | 0.67 |
| 100 | 4.58 |
| 101 | 0.38 |
| 102 | 0.19 |
| 103 | 0.24 |
| 104 | 0.1 |
| 105 | 0.72 |
| 106 | 0.61 |
| 107 | 0.93 |
| 108 | 1.06 |
| 109 | 0.69 |
| 110 | 1.05 |
| 111 | 7.13 |
| 112 | 1.03 |
| 113 | 0.62 |
| 114 | 0.2 |
| 115 | 0.43 |
| 116 | 0.31 |
| 117 | 0.47 |
| 118 | 0.2 |
| 119 | 0.23 |
| 120 | 0.31 |
| 121 | 0.28 |
| 122 | 0.34 |
| 123 | 0.24 |
| 124 | 0.2 |
| 125 | 2.17 |
| 126 | 1.48 |
| 127 | 1.82 |
| 128 | 1.8 |
| 129 | 2.31 |
| 130 | 0.75 |
| 131 | 2.01 |
| 132 | 1.6 |
| 133 | 0.5 |
| 134 | 0.71 |
| 135 | 0.51 |
| 136 | 0.41 |
| 137 | 3.04 |
| 138 | 1.87 |
| 139 | 0.26 |
| 140 | 0.29 |
| 141 | 0.43 |
| 142 | 0.23 |
| 143 | 0.33 |
| 144 | 0.42 |
| 145 | 0.29 |
| 146 | 0.17 |
| 147 | 1.18 |
| 148 | 1.6 |
| 149 | 1.77 |
| 150 | 1.26 |
| 151 | 0.49 |
| 152 | 1.65 |
| 153 | 0.32 |
| 154 | 0.38 |
| 155 | 0.39 |
| 156 | 0.62 |
| 157 | 0.81 |
| 158 | 0.45 |
| 159 | 4.28 |
| 160 | 5.05 |
| 161 | 7.41 |
| 162 | 7.55 |
| 163 | 8.05 |
| 164 | 1.39 |
| 165 | 0.55 |
| 166 | 0.69 |
| 167 | 0.37 |
| 168 | 0.188 |
| 169 | 0.14 |
| 170 | 0.31 |
| 171 | 0.19 |
| 172 | 0.33 |
| 173 | 0.24 |
| 174 | 0.35 |
| 175 | 0.28 |
| 176 | 0.24 |
| 177 | 0.25 |
| 178 | 0.22 |
| 179 | 0.19 |
| 180 | 1.32 |

TABLE 3

| Example | IC₅₀ (nM) |
|---|---|
| 181 | 1.85 |
| 182 | 3.1 |
| 183 | 1.6 |
| 184 | 0.3 |
| 185 | 0.3 |
| 186 | 0.46 |
| 187 | 0.51 |
| 188 | 0.84 |
| 189 | 1.04 |
| 190 | 0.72 |
| 191 | 0.62 |
| 192 | 0.53 |
| 193 | 0.78 |
| 194 | 0.41 |
| 195 | 1.29 |
| 196 | 0.43 |
| 197 | 0.2 |
| 198 | 0.34 |
| 199 | 0.192 |

From the results of Tables 1 to 3, it can be seen that the compounds of the present invention exhibit excellent inhibitory activity against GCS.

Experimental Example 2: Evaluation of inhibitory activities against GM1 production GM1, which is the final product of sphingolipid metabolism, is expressed on the cell membrane and thus the detection thereof is easy. In addition, the amount of GM1 represents the conversion of ceramide to glucosylceramide. Therefore, the inhibitory activities of the compounds of the present invention against GM1 production were evaluated, as follows, according to the method described in the known literature (Dijkhuis et al., Gangliosides do not affect ABC transporter function in human neuroblastoma cells. The Journal of Lipid Research 47 (2006). 1187-1195). Ibiglustat, known as a GCS inhibitor, was used as a control.

### (1) Materials

Jurkat cells, Clone E6-1 (ATCC, TIB-152)
Cholera toxin subunit B (CTB), FITC (Sigma, C1655)
Ibiglustat (Shanghai Systeam Biochem Co., ltd, Genz-682452)
DMSO (Sigma, D2650)
Fixation buffer (BD, 554655)
RPMI 1640 (Gibco, A1049101)
FBS (Gibco, 16000-044)
Antibiotic-Antimycotic (100×) (Gibco, 15240-122)
FACS Sheath Fluid (BD, 342003)

Jurkat cells were cultured in RPMI 1640 medium supplemented with 10% FBS and 1× Antibiotic-Antimycotic. A washing solution was prepared by adding 10 ml of FBS to 490 ml of FACS Sheath Fluid. The CTB-FITC solution was prepared by diluting the CTB-FITC stock solution (10 mg/ml) with the washing solution to a final concentration of 2 μg/ml.

### (2) Protocol

A cell suspension (1×10⁵ cells/ml) was prepared with the culture medium (RPMI 1640 medium supplemented with 10% FBS and 1× Antibiotic-Antimycotic). The cell suspension (200 μl) was added to each well of a 96-well plate (20,000 cells/well) and then the compounds were treated thereto in a final concentration of 0.05 to 3000 nM (3 fold, 11 points) per well. Each mixture was incubated in a CO₂ incubator at 37ºC for 72 hours. After centrifuging at 1500 rpm for 3 minutes to remove the medium, the cells were resuspended in 200 μl of the washing solution per well. After centrifuging at 1500 rpm for 3 minutes to remove the washing solution, the cells were resuspended in 200 μl of the 2 μg/ml CTB-FITC solution. The obtained suspension was incubated at 4ºC for 60 minutes, while not being exposed to light. After centrifuging at 1500 rpm for 3 minutes to remove the CTB-FITC solution, the cells were washed with 200 μl of the washing solution. The washing process was additionally repeated twice. The washed plate was centrifuged at 1500 rpm for 3 minutes to remove the washing solution and then the cells were completely resuspended in 200 μl of the fixation buffer. IC₅₀ was determined from the values obtained by quantifying FITC fluorescences with Guava™ easyCyte 5HT (Merck Milipore, 0500-4005).

Data analysis was performed by the following equations.

% MFI(median fluorescence intensity)=(fluorescence value of drug-treated group/fluorescence value of DMSO-treated group)×100%

% Cells=(cell concentration in the well/cell concentration in the DMSO-treated group)×100%

% MFI and % cell data were analyzed with the software GraphPad Prism (Ver 5.01) to calculate IC₅₀ values. The results are shown in Tables 4 to 6 below.

TABLE 4

| Example | IC₅₀ (nM) |
|---|---|
| 1 | 4.53 |
| 2 | 3.75 |
| 3 | 1.24 |
| 4 | 3.74 |
| 5 | 4.14 |
| 6 | 9.97 |
| 7 | 13.71 |
| 8 | 1.09 |
| 9 | 22.12 |
| 10 | 10.26 |
| 11 | 23.4 |
| 12 | 5.94 |
| 13 | 4.15 |
| 14 | 48.87 |
| 15 | 40.52 |
| 16 | 28.72 |
| 17 | 20.64 |
| 18 | 50.7 |
| 19 | 55.98 |
| 20 | 18.36 |
| 21 | 20.85 |
| 22 | 38.07 |
| 23 | 48.87 |
| 24 | 40.52 |
| 25 | 28.72 |
| 26 | 20.64 |
| 27 | 50.7 |
| 28 | 55.98 |
| 29 | 18.36 |
| 30 | 20.85 |
| 31 | 38.07 |
| 32 | 4.23 |
| 33 | 7.3 |
| 34 | 2.3 |
| 35 | 3.3 |
| 36 | 0.7 |
| 37 | 2.2 |
| 38 | 3.2 |
| 39 | 1.1 |
| 40 | 1.6 |
| 41 | 13.6 |
| 42 | 2.6 |
| 43 | 6.4 |
| 44 | 2.3 |
| 45 | 6 |
| 46 | 11.6 |
| 47 | 7.7 |
| 48 | 13.9 |
| 49 | 9.4 |
| 50 | 2.2 |
| 51 | 9.1 |
| 52 | 36.7 |
| 53 | 4.1 |
| 54 | 8.6 |
| 55 | 2.2 |
| 56 | 10.7 |
| 57 | 0.99 |
| 58 | 5.74 |
| 59 | 4.3 |
| 60 | 1 |
| 61 | 10.55 |
| 62 | 3.4 |
| 63 | 20.6 |
| 64 | 11.6 |
| 65 | 8.8 |
| 66 | 5.04 |
| 67 | 5.3 |
| 68 | 1.54 |
| 69 | 1.1 |
| 70 | 0.6 |
| 71 | 12.7 |
| 72 | 15.9 |
| 73 | 10.85 |
| 74 | 19.4 |
| 75 | 14.83 |
| 76 | 33.4 |
| 77 | 23.7 |
| 78 | 17.6 |
| 79 | 6 |
| 80 | 51.04 |
| 81 | 18.7 |
| 82 | 20.3 |
| 83 | 10.4 |
| 84 | 17.24 |
| 85 | 66.69 |
| 86 | 45.8 |
| 87 | 36.2 |
| 88 | 21.1 |
| 89 | 43.5 |
| 90 | 26.35 |

TABLE 5

| Example | IC₅₀ (nM) |
|---|---|
| 91 | 10.74 |
| 92 | 1.16 |
| 93 | 5.6 |
| 94 | 12.1 |
| 95 | 18.2 |
| 96 | 0.76 |
| 97 | 12.98 |
| 98 | 0.2 |
| 99 | 8.84 |
| 100 | 16.25 |
| 101 | 4.67 |
| 102 | 1.6 |
| 103 | 5.6 |
| 104 | 1.4 |
| 105 | 8.2 |
| 106 | 2.3 |
| 107 | 16 |
| 108 | 31.6 |
| 109 | 11.5 |
| 110 | 28 |
| 111 | 76.2 |
| 112 | 10.6 |
| 113 | 11.5 |
| 114 | 1.7 |
| 115 | 5.09 |
| 116 | 3.82 |
| 117 | 4.42 |
| 118 | 1.55 |
| 119 | 4.95 |
| 120 | 7.66 |
| 121 | 2.79 |
| 122 | 3.66 |
| 123 | 1.26 |
| 124 | 1.13 |
| 125 | 10.8 |
| 126 | 18 |
| 127 | 8.02 |
| 128 | 6.49 |
| 129 | 8.11 |
| 130 | 1.53 |
| 131 | 6.47 |
| 132 | 7.62 |
| 133 | 2.63 |
| 134 | 3.8 |
| 135 | 1.46 |
| 136 | 0.87 |
| 137 | 12.8 |
| 138 | 28 |
| 139 | 3.01 |
| 140 | 2.5 |
| 141 | 2.77 |
| 142 | 1.4 |
| 143 | 4.2 |
| 144 | 2.3 |
| 145 | 0.3 |
| 146 | 0.68 |
| 147 | 9.4 |
| 148 | 8.86 |
| 149 | 14.6 |
| 150 | 3.27 |
| 151 | 5.2 |
| 152 | 8.3 |
| 153 | 2.1 |
| 154 | 2.9 |
| 155 | 0.7 |
| 156 | 1.9 |
| 157 | 2.64 |
| 158 | 1.1 |
| 159 | 1.7 |
| 160 | 3 |
| 161 | 4.5 |
| 162 | 1.4 |
| 163 | 1.3 |
| 164 | 8.4 |
| 165 | 2.51 |
| 166 | 5.9 |
| 167 | 3.16 |
| 168 | 0.23 |
| 169 | 1.7 |
| 170 | 1.4 |
| 171 | 1.4 |
| 172 | 0.86 |
| 173 | 0.3 |
| 174 | 0.9 |
| 175 | 3.8 |
| 176 | 2.1 |
| 177 | 0.43 |
| 178 | 0.4 |
| 179 | 0.22 |
| 180 | 8 |

TABLE 6

| Example | IC₅₀ (nM) |
|---|---|
| 181 | 7.1 |
| 182 | 12.9 |
| 183 | 7.6 |
| 184 | 2.29 |
| 185 | 3.6 |
| 186 | 0.71 |
| 187 | 1.3 |
| 188 | 2.7 |
| 189 | 6 |
| 190 | 1.9 |
| 191 | 2.28 |
| 192 | 2.9 |
| 193 | 6.75 |
| 194 | 4.6 |
| 195 | 8.3 |
| 196 | 6 |
| 197 | 0.94 |
| 198 | 3.2 |
| 199 | 0.03 |

From the results of Tables 4 to 6, it can be seen that the compounds of the present invention exhibit excellent inhibitory activity against GM1 production.

Experimental Example 3: Evaluation of blood-brain barrier (BBB) permeability through oral administration

The blood-brain barrier (BBB) permeability was evaluated in mice, with respect to the compounds according to the present invention and the control (ibiglustat). Each compound of the present invention and the control were suspended in 0.5% methyl cellulose containing 0.2% Tween 80 and then orally administered to mice at a dose of 10 or 30 mg/10 ml/kg. After the whole blood was collected from the heart of each mouse at the time of 4 hours after the administration, it was placed in a lithium heparin tube and immediately centrifuged to obtain the plasma. After the exsanguination was carried out through abdominal vena cava incision, the forebrain was taken by dissection of the head. Both of the forebrain and the plasma were stored in a −80° C. freezer until the analysis of drug concentrations therein. For the analysis of drug concentrations therein, the forebrain was immersed in distilled water and then homogenized with a homogenizer to obtain a homogenate. Each plasma and homogenate was treated with acetonitrile to precipitate proteins, vortexed for 15 seconds, and then centrifuged (12700 rpm, 10 min, 4° C.). The obtained supernatant was diluted with an 80% aqueous solution of acetonitrile containing 0.1% formic acid and then analyzed with an LC-MS/MS instrument. LC-MS/MS analysis was carried out with a reversed-phase column, using distilled water containing 0.1% formic acid and acetonitrile as mobile phases. Each eluted material was detected through a mass spectrometer to calculate the drug concentrations thereof. The brain-plasma drug ratio (B/P ratio) was calculated from the drug concentrations in each tissue to evaluate the blood-brain barrier (BBB) permeability. The results are shown in Tables 7 and 8 below.

TABLE 7

| | Mean drug | Mean drug | | |
|---|---|---|---|---|
| | concentrations | concentrations | Mean brain- | |
| | in plasma | in brain tissue | plasma drug | |
| | at 4 hr | at 4 hr | ratio | Dose |
| Example | (ng/mL) | (ng/g) | at 4 hr | (mg/kg) |
| 9 | 402.9 | 2749.8 | 6.836 | 30 |
| 10 | 1090.3 | 6104.9 | 5.615 | 30 |
| 11 | 265.1 | 2410.8 | 9.12 | 10 |
| 14 | 781.1 | 3071.5 | 3.916 | 30 |
| 15 | 1294.4 | 6034.7 | 4.661 | 30 |
| 16 | 159.7 | 1277.2 | 8.015 | 30 |
| 18 | 1134.5 | 3948.1 | 3.472 | 30 |
| 20 | 577.7 | 4065.6 | 7.122 | 30 |
| 21 | 1482.1 | 9505.2 | 6.482 | 30 |
| 22 | 1315.3 | 4404.1 | 3.37 | 30 |
| 32 | 1340.2 | 5650 | 4.227 | 30 |
| 34 | 766.9 | 10682 | 13.958 | 30 |
| 35 | 1076.8 | 2007 | 1.855 | 30 |
| 36 | 1825.3 | 6652.1 | 3.648 | 30 |
| 38 | 669.7 | 3642 | 5.598 | 30 |
| 39 | 564.3 | 5508.1 | 9.851 | 30 |
| 43 | 1563.9 | 5724.1 | 3.653 | 30 |
| 44 | 1847.4 | 8862.2 | 4.789 | 30 |
| 45 | 857.8 | 7815.2 | 9.086 | 30 |
| 57 | 63.7 | 1331.7 | 20.895 | 30 |
| 59 | 436.3 | 4123.6 | 9.7 | 30 |
| 61 | 438.7 | 4884.8 | 11.147 | 30 |
| 62 | 433.5 | 2963.4 | 6.83 | 30 |
| 63 | 973.7 | 1355.1 | 1.398 | 30 |
| 64 | 233.5 | 1041 | 4.434 | 30 |
| 65 | 810.7 | 4078.6 | 5.036 | 30 |
| 66 | 719.3 | 5492.4 | 7.85 | 30 |
| 67 | 902.8 | 3674.9 | 4.111 | 30 |
| 68 | 1417.2 | 13523.3 | 9.765 | 30 |
| 69 | 432.7 | 4144.9 | 9.842 | 30 |

TABLE 8

| | Mean drug | Mean drug | | |
|---|---|---|---|---|
| | concentrations | concentrations | Mean brain- | |
| | in plasma | in brain tissue | plasma drug | |
| | at 4 hr | at 4 hr | ratio | Dose |
| Example | (ng/mL) | (ng/g) | at 4 hr | (mg/kg) |
| 82 | 895.5 | 5523.3 | 6.321 | 30 |
| 83 | 1165.8 | 7365.2 | 6.295 | 30 |
| 84 | 683.2 | 4944.5 | 7.23 | 30 |
| 86 | 851.6 | 4794.7 | 5.654 | 30 |
| 87 | 113.5 | 971.6 | 8.573 | 30 |
| 89 | 750.9 | 4041.5 | 5.417 | 30 |
| 90 | 874.1 | 6484.8 | 7.424 | 30 |
| 92 | 544.8 | 2234.9 | 4.131 | 30 |
| 96 | 863.7 | 5359.9 | 6.237 | 30 |
| 103 | 1701.8 | 4680.4 | 2.761 | 30 |
| 104 | 1380.7 | 5175.1 | 3.757 | 30 |
| 148 | 951.4 | 1682.4 | 1.825 | 30 |
| 150 | 1065 | 1520.5 | 1.471 | 30 |
| 151 | 1196.4 | 2441.3 | 2.04 | 30 |
| 158 | 1019.3 | 3725 | 3.674 | 30 |
| 161 | 1239.9 | 4843.5 | 3.966 | 30 |
| 162 | 967.2 | 3570.8 | 3.71 | 30 |
| 165 | 852.5 | 2402.5 | 2.832 | 30 |
| 168 | 2888.3 | 3367 | 1.175 | 30 |
| 171 | 747.9 | 2898 | 3.876 | 30 |
| 176 | 1360.1 | 4863 | 3.575 | 30 |
| 177 | 880.7 | 4788.9 | 5.444 | 30 |
| 180 | 1136.5 | 1507.4 | 1.345 | 30 |
| 181 | 510.2 | 848.3 | 1.649 | 30 |
| 193 | 871.3 | 3017.5 | 3.464 | 30 |
| 194 | 1258.4 | 2103.3 | 1.671 | 30 |
| 197 | 1879.6 | 6690.1 | 3.557 | 30 |
| 198 | 1476.4 | 5121.7 | 3.458 | 30 |
| 199 | 2327.6 | 2689 | 1.165 | 30 |
| Control | 2644 | 1321.9 | 0.501 | 30 |

From the results of Tables 7 and 8, it can be seen that the compounds of the present invention exhibit excellent blood-brain barrier permeability.

### Experimental Example 4: Single Crystal X-Ray Diffraction (SC-XRD)

In order to identify the absolute configuration of the compound prepared in Preparation 1, i.e., (S)-quinuclidin-3-yl((R)-5-bromo-2,2-dimethyl-2,3-dihydro-1H-inden-1-yl)carbamate, the compound of Preparation 1 was prepared in the form of single crystal, followed by analyzing the structure thereof through SC-XRD assay.

Test solutions were prepared so that the concentration thereof at 30° C. was about 0.1 g/mL in the solvent mixtures (isopropyl acetate/n-hexane), so as to screen the crystallization conditions for forming a single crystal of the compound prepared in Preparation 1 (i.e., (S)-quinuclidin-3-yl((R)-5-bromo-2,2-dimethyl-2,3-dihydro-1H-inden-1-yl)carbamate). As a single crystal manufacturing method, cooling-crystallization was employed. Finally, the crystal obtained by employing isopropyl acetate/n-hexane=1/1 as a solvent condition and by crystallizing through cooling from 30° ° C. to 20° ° C. was subject to optical microscope observation.

The crystal prepared under the above conditions was analyzed using single crystal X-ray diffraction (SC-XRD). The instrument and analysis conditions used are shown in Table 9 below.

TABLE 9

| | |
|---|---|
| Instrument and model | Bruker SMART APEX-II |
| Source | Mo Kα (λ = 0.71073 Å) |
| Operation voltage/current | 50 kV/30 mA |
| Monochromator | ‘graphite crystal’ |
| Detector | Charge-Coupled Device (CCD) |
| Exposure time | 12 sec/picture |
| Operating temperature | Low Temperature 100(1) K |
| Theta range for data collection | 2.195 to 26.019 |
| Total data collection time | 3 hours |

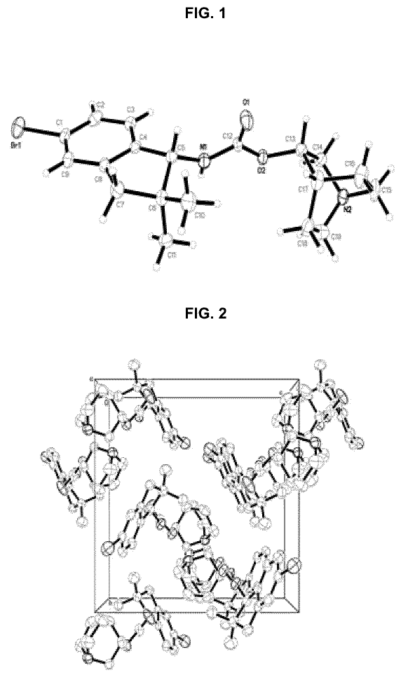

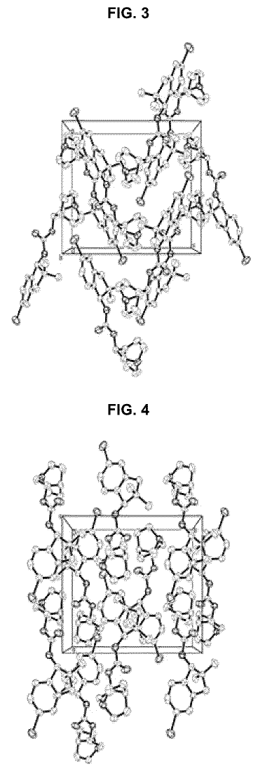

The results of single crystal X-ray diffraction (SC-XRD) analysis are shown in Table 10 below. In addition, the single crystal X-ray structure obtained from the SC-XRD analysis results is shown in FIG. 1 and the packing crystal structures along the a-axis, b-axis, and c-axis are shown in FIGS. 2 to 4.

TABLE 10

| Empirical formula | C19 H25 Br N2 O2 | |
|---|---|---|
| Formula weight | 393.32 | |
| Temperature | 100(1) K | |
| Wavelength | 0.71073 Å | |
| Crystal system | Orthorhombic | |
| Space group | P212121 | |
| Unit cell dimensions | a = 10.8809(18) Å | α = 90° |
| | b = 12.986(2) Å | β = 90° |
| | c = 13.260(3) Å | γ = 90° |

| | |
|---|---|
| Volume | 1873.6(6) Å³ |
| Z | 4 |
| Density (calculated) | 1.394 Mg/m³ |
| Absorption coefficient | 2.207 mm⁻¹ |
| F(000) | 816 |
| Crystal size | 0.300 × 0.200 × 0.160 mm³ |
| Theta range for data collection | 2.195 to 26.019° |
| Index ranges | −13 <= h <= 12, −16 <= |
| | k <= 15, −16 <= l <= 10 |
| Reflections collected | 9006 |
| Independent reflections | 3650 [R(int) = 0.0342] |
| Completeness to theta = 25.242° | 99.70% |
| Absorption correction | Semi-empirical from equivalents |
| Max. and min. transmission | 0.72 and 0.59 |
| Refinement method | Full-matrix least-squares on F² |
| Data/restraints/parameters | 3650/0/219 |
| Goodness-of-fit on F² | 1.089 |
| Final R indices [I > 2sigma(I)] | R1 = 0.0460, wR2 = 0.0876 |
| R indices (all data) | R1 = 0.0573, wR2 = 0.1001 |
| Absolute structure parameter | −0.007(8) |
| Extinction coefficient | n/a |
| Largest diff. peak and hole | 0.542 and −0.335 e · Å⁻³ |

From the results of Table 10 and FIGS. 1 to 4, It can be confirmed that the compound prepared in Preparation 1 has the absolute configuration of (S)-quinuclidin-3-yl((R)-5-bromo-2,2-dimethyl-2,3-dihydro-1H-inden-1-yl)carbamate.

1. A compound of Formula 1 or pharmaceutically acceptable salt thereof: 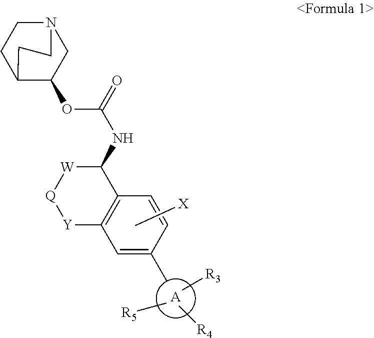
wherein,
W and Q are, independently each other, —CR₁R₂—,
Y is a bond, —CR₁′R₂′—; or —O—,
R₁ and R₂ are, independently each other, hydrogen; halogen; C₁˜C₆ alkyl; C₁˜C₆ alkyl having a nitrogen, oxygen, or sulfur atom; C₃˜C₁₀ cycloalkyl; 3— to 12-membered heterocyclic; or
C₁˜C₆ alkoxy; or R₁ and R₂ form C₃˜C₁₀ cycloalkyl together with the carbon atom to which they are attached,
R₁′ and R₂′ are, independently each other, hydrogen; halogen; C₁˜C₆ alkyl; C₁˜C₆ alkyl having a nitrogen, oxygen, or sulfur atom; C₃˜C₁₀ cycloalkyl; 3— to 12-membered heterocyclic; or
C₁˜C₆ alkoxy; or R₁ and R₂ form C₃˜C₁₀ cycloalkyl together with the carbon atom to which they are attached,
X is hydrogen; halogen; C₁˜C₆ alkyl; C₁˜C₆ alkyl substituted with 1 to 3 halogens; C₁˜C₆ alkyl having a nitrogen, oxygen, or sulfur atom; C₁˜C₆ alkoxy; or C₁˜C₆ alkoxy substituted with 1 to 3 halogens,
A ring is 6— to 12-membered aryl; 5— to 12-membered heteroaryl; C₃˜C₁₀ cycloalkyl; or 3—to 12-membered heterocyclic, and
R₃, R₄, and R₅ are, independently each other, hydrogen; cyano; halogen; C₁˜C₆ alkyl; C₁˜C₆ alkoxy-C₁˜C₆ alkyl; C₁˜C₆ alkyl substituted with 1 to 3 halogens; C₃˜C₁₀ cycloalkyl; 3— to 12-membered heterocyclic; C₁˜C₆ alkoxy; C₁˜C₆ alkoxy substituted with 1 to 3 halogens; C₁˜C₆ alkoxy-C₁˜C₆ alkoxy; morpholinyl-C₁˜C₆ alkoxy; mono- or di-C₁˜C₆ alkylamino-C₁˜C₆ alkoxy; C₃˜C₁₀ cycloalkyl-C₁˜C₆ alkoxy; C₁˜C₆ alkylthio; amino; mono- or di-C₁˜C₆ alkylamino; C₁˜C₆ alkylcarbonyl; hydroxy; or nitro.

2. The compound or pharmaceutically acceptable salt thereof as claimed in claim 1, wherein W is —C(CH₃)₂ —.

3. The compound or pharmaceutically acceptable salt thereof as claimed in claim 1, wherein Y is a bond.

4. The compound or pharmaceutically acceptable salt thereof as claimed in claim 1, wherein Y is —CH₂—.

5. The compound or pharmaceutically acceptable salt thereof as claimed in claim 1, wherein Y is —O—.

6. The compound or pharmaceutically acceptable salt thereof as claimed in claim 1, wherein X is hydrogen, halogen, C₁˜C₆ alkyl, or C₁˜C₆ alkoxy.

7. The compound or pharmaceutically acceptable salt thereof as claimed in claim 1, wherein the A ring is phenyl or pyridinyl.

8. The compound or pharmaceutically acceptable salt thereof as claimed in claim 1, wherein R₃, R₄ and R₅ are, independently each other, hydrogen, halogen, C₁˜C₆ alkyl, C₁˜C₆ alkoxy, C₁˜C₆ alkoxy substituted with 1 to 3 halogens, or C₃˜C₁₀ cycloalkyl-C₁˜C₆ alkoxy.

9. The compound or pharmaceutically acceptable salt thereof as claimed in claim 1, wherein
W is —C(CH₃)₂—,
Q is —CH₂—,
Y is a bond,
X is hydrogen, halogen, C₁˜C₆ alkyl, or C₁˜C₆ alkoxy,
the A ring is phenyl or pyridinyl, and
R₃, R₄ and R₅ are, independently each other, hydrogen, halogen, C₁˜C₆ alkyl, C₁˜C₆ alkoxy, C₁˜C₆ alkoxy substituted with 1 to 3 halogens, or C₃˜C₁₀ cycloalkyl-C₁˜C₆ alkoxy.

10. The compound or pharmaceutically acceptable salt thereof as claimed in claim 1, wherein
W is —C(CH₃)₂—,
Q is —CH₂—,
Y is —CH₂—,
X is hydrogen, halogen, C₁˜C₆ alkyl, or C₁˜C₆ alkoxy,
the A ring is phenyl or pyridinyl, and
R₃, R₄ and R₅ are, independently each other, hydrogen, halogen, C₁˜C₆ alkyl, or C₁˜C₆ alkoxy.

11. The compound or pharmaceutically acceptable salt thereof as claimed in claim 1, wherein
W is —C(CH₃)₂—,
Q is —CH₂—,
Y is —O—,
X is hydrogen, halogen, C₁˜C₆ alkyl, or C₁˜C₆ alkoxy,
the A ring is phenyl or pyridinyl, and
R₃, R₄ and R₅ are, independently each other, hydrogen, halogen, C₁˜C₆ alkyl, or C₁˜C₆ alkoxy.

12. The compound or pharmaceutically acceptable salt thereof as claimed in claim 1, which is selected from the group consisting of:
(S)-quinuclidin-3-yl((R)-5-(3-ethylphenyl)-2,2-dimethyl-2,3-dihydro-1H-inden-1-yl)carbamate;
(S)-quinuclidin-3-yl((R)-5-(3-isopropylphenyl)-2,2-dimethyl-2,3-dihydro-1H-inden-1-yl)carbamate;
(S)-quinuclidin-3-yl((R)-5-(4-butylphenyl)-2,2-dimethyl-2,3-dihydro-1H-inden-1-yl)carbamate;
(S)-quinuclidin-3-yl((R)-2,2-dimethyl-5-(4-propylphenyl)-2,3-dihydro-1H-inden-1-yl)carbamate;
(S)-quinuclidin-3-yl((R)-5-(4-isobutylphenyl)-2,2-dimethyl-2,3-dihydro-1H-inden-1-yl)carbamate;
(S)-quinuclidin-3-yl((R)-5-(4-(tert-butyl)phenyl)-2,2-dimethyl-2,3-dihydro-1H-inden-1-yl)carbamate;
(S)-quinuclidin-3-yl((R)-5-(4-ethoxyphenyl)-2,2-dimethyl-2,3-dihydro-1H-inden-1-yl)carbamate;
(S)-quinuclidin-3-yl((R)-5-(4-isobutoxyphenyl)-2,2-dimethyl-2,3-dihydro-1H-inden-1-yl)carbamate;
(S)-quinuclidin-3-yl((R)-5-(3,5-dimethyl-4-propoxyphenyl)-2,2-dimethyl-2,3-dihydro-1H-inden-1-yl)carbamate;
(S)-quinuclidin-3-yl((R)-5-(4-isopropoxy-3,5-dimethylphenyl)-2,2-dimethyl-2,3-dihydro-1H-inden-1-yl)carbamate;
(S)-quinuclidin-3-yl((R)-5-(3-chloro-4-isopropoxyphenyl)-2,2-dimethyl-2,3-dihydro-1H-inden-1-yl)carbamate;
(S)-quinuclidin-3-yl((R)-5-(2-fluoro-4-methoxyphenyl)-2,2-dimethyl-2,3-dihydro-1H-inden-1-yl)carbamate;
(S)-quinuclidin-3-yl((R)-5-(6-(cyclopropylmethoxy)pyridin-3-yl)-2,2-dimethyl-2,3-dihydro-1H-inden-1-yl)carbamate;
(S)-quinuclidin-3-yl((R)-5-(3-chloro-4-methoxyphenyl)-2,2-dimethyl-2,3-dihydro-1H-inden-1-yl)carbamate;
(S)-quinuclidin-3-yl((R)-5-(3-chloro-4-ethoxyphenyl)-2,2-dimethyl-2,3-dihydro-1H-inden-1-yl)carbamate;
(S)-quinuclidin-3-yl((R)-5-(3-chloro-4-propoxyphenyl)-2,2-dimethyl-2,3-dihydro-1H-inden-1-yl)carbamate;
(S)-quinuclidin-3-yl((R)-5-(4-butoxy-3-chlorophenyl)-2,2-dimethyl-2,3-dihydro-1H-inden-1-yl)carbamate;
(S)-quinuclidin-3-yl((R)-5-(3-fluoro-4-methoxyphenyl)-2,2-dimethyl-2,3-dihydro-1H-inden-1-yl)carbamate;
(S)-quinuclidin-3-yl((R)-5-(4-ethoxy-3-fluorophenyl)-2,2-dimethyl-2,3-dihydro-1H-inden-1-yl)carbamate;
(S)-quinuclidin-3-yl((R)-5-(3-fluoro-4-propoxyphenyl)-2,2-dimethyl-2,3-dihydro-1H-inden-1-yl)carbamate;
(S)-quinuclidin-3-yl((R)-5-(3-fluoro-4-isopropoxyphenyl)-2,2-dimethyl-2,3-dihydro-1H-inden-1-yl)carbamate;
(S)-quinuclidin-3-yl((R)-5-(4-ethoxy-3,5-dimethylphenyl)-2,2-dimethyl-2,3-dihydro-1H-inden-1-yl)carbamate;
(S)-quinuclidin-3-yl((R)-5-(3-chloro-4-methoxyphenyl)-2,2-dimethyl-2,3-dihydro-1H-inden-1-yl)carbamate;
(S)-quinuclidin-3-yl((R)-5-(3-chloro-4-ethoxyphenyl)-2,2-dimethyl-2,3-dihydro-1H-inden-1-yl)carbamate;
(S)-quinuclidin-3-yl((R)-5-(3-chloro-4-propoxyphenyl)-2,2-dimethyl-2,3-dihydro-1H-inden-1-yl)carbamate;
(S)-quinuclidin-3-yl((R)-5-(4-butoxy-3-chlorophenyl)-2,2-dimethyl-2,3-dihydro-1H-inden-1-yl)carbamate;
(S)-quinuclidin-3-yl((R)-5-(3-fluoro-4-methoxyphenyl)-2,2-dimethyl-2,3-dihydro-1H-inden-1-yl)carbamate;
(S)-quinuclidin-3-yl((R)-5-(4-ethoxy-3-fluorophenyl)-2,2-dimethyl-2,3-dihydro-1H-inden-1-yl)carbamate;
(S)-quinuclidin-3-yl((R)-5-(3-fluoro-4-propoxyphenyl)-2,2-dimethyl-2,3-dihydro-1H-inden-1-yl)carbamate;
(S)-quinuclidin-3-yl((R)-5-(3-fluoro-4-isopropoxyphenyl)-2,2-dimethyl-2,3-dihydro-1H-inden-1-yl)carbamate;
(S)-quinuclidin-3-yl((R)-5-(4-ethoxy-3,5-dimethylphenyl)-2,2-dimethyl-2,3-dihydro-1H-inden-1-yl)carbamate;
(S)-quinuclidin-3-yl((R)-5-(2-chloro-4-isopropoxyphenyl)-2,2-dimethyl-2,3-dihydro-1H-inden-1-yl)carbamate;
(S)-quinuclidin-3-yl((R)-5-(4-ethyl-3-fluorophenyl)-2,2-dimethyl-2,3-dihydro-1H-inden-1-yl)carbamate;
(S)-quinuclidin-3-yl((R)-5-(4-isopropoxy-3-methylphenyl)-2,2-dimethyl-2,3-dihydro-1H-inden-1-yl)carbamate;
(S)-quinuclidin-3-yl((R)-5-(4-isopropoxy-3-methoxyphenyl)-2,2-dimethyl-2,3-dihydro-1H-inden-1-yl)carbamate;
(S)-quinuclidin-3-yl((R)-5-(4-isopropoxy-2-methylphenyl)-2,2-dimethyl-2,3-dihydro-1H-inden-1-yl)carbamate;
(S)-quinuclidin-3-yl((R)-5-(3,5-difluoro-4-isopropoxyphenyl)-2,2-dimethyl-2,3-dihydro-1H-inden-1-yl)carbamate;
(S)-quinuclidin-3-yl((R)-5-(2-chloro-4-(cyclopropylmethoxy)phenyl)-2,2-dimethyl-2,3-dihydro-1H-inden-1-yl)carbamate;
(S)-quinuclidin-3-yl((R)-5-(3-chloro-4-(cyclopropylmethoxy)phenyl)-2,2-dimethyl-2,3-dihydro-1H-inden-1-yl)carbamate;
(S)-quinuclidin-3-yl((R)-5-(3,5-dichloro-4-isopropoxyphenyl)-2,2-dimethyl-2,3-dihydro-1H-inden-1-yl)carbamate;
(S)-quinuclidin-3-yl((R)-5-(4-(tert-butoxy)phenyl)-2,2-dimethyl-2,3-dihydro-1H-inden-1-yl)carbamate;
(S)-quinuclidin-3-yl((R)-5-(4-(tert-butoxy)-2-chlorophenyl)-2,2-dimethyl-2,3-dihydro-1H-inden-1-yl)carbamate;
(S)-quinuclidin-3-yl((R)-5-(2-chloro-4-methoxyphenyl)-2,2-dimethyl-2,3-dihydro-1H-inden-1-yl)carbamate;
(S)-quinuclidin-3-yl((R)-5-(2-chloro-4-ethoxyphenyl)-2,2-dimethyl-2,3-dihydro-1H-inden-1-yl)carbamate;
(S)-quinuclidin-3-yl((R)-5-(3-chloro-4-methylphenyl)-2,2-dimethyl-2,3-dihydro-1H-inden-1-yl)carbamate;
(S)-quinuclidin-3-yl((R)-5-(4-ethoxy-3-methylphenyl)-2,2-dimethyl-2,3-dihydro-1H-inden-1-yl)carbamate;
(S)-quinuclidin-3-yl((R)-2,2-dimethyl-5-(3-methyl-4-propoxyphenyl)-2,3-dihydro-1H-inden-1-yl)carbamate;
(S)-quinuclidin-3-yl((R)-5-(4-(difluoromethoxy)-3-methylphenyl)-2,2-dimethyl-2,3-dihydro-1H-inden-1-yl)carbamate;
(S)-quinuclidin-3-yl((R)-5-(3-fluoro-5-isopropoxyphenyl)-2,2-dimethyl-2,3-dihydro-1H-inden-1-yl)carbamate;
(S)-quinuclidin-3-yl((R)-5-(3-fluoro-5-isobutoxyphenyl)-2,2-dimethyl-2,3-dihydro-1H-inden-1-yl)carbamate;
(S)-quinuclidin-3-yl((R)-5-(3-fluoro-5-(2,2,2-trifluoroethoxy)phenyl)-2,2-dimethyl-2,3-dihydro-1H-inden-1-yl)carbamate;
(S)-quinuclidin-3-yl((R)-5-(4-chloro-3-isopropoxyphenyl)-2,2-dimethyl-2,3-dihydro-1H-inden-1-yl)carbamate;
(S)-quinuclidin-3-yl((R)-5-(3-chloro-5-isobutoxyphenyl)-2,2-dimethyl-2,3-dihydro-1H-inden-1-yl)carbamate;
(S)-quinuclidin-3-yl((R)-5-(3-chloro-5-isopropoxyphenyl)-2,2-dimethyl-2,3-dihydro-1H-inden-1-yl)carbamate;
(S)-quinuclidin-3-yl((R)-5-(2-chloro-4-(2,2,2-trifluoroethoxy)phenyl)-2,2-dimethyl-2,3-dihydro-1H-inden-1-yl)carbamate;
(S)-quinuclidin-3-yl((R)-5-(4-ethoxyphenyl)-6-fluoro-2,2-dimethyl-2,3-dihydro-1H-inden-1-yl)carbamate;
(S)-quinuclidin-3-yl((R)-6-fluoro-5-(4-isobutoxyphenyl)-2,2-dimethyl-2,3-dihydro-1H-inden-1-yl)carbamate;
(S)-quinuclidin-3-yl((R)-5-(4-ethylphenyl)-6-fluoro-2,2-dimethyl-2,3-dihydro-1H-inden-1-yl)carbamate;
(S)-quinuclidin-3-yl((R)-6-fluoro-5-(4-isopropylphenyl)-2,2-dimethyl-2,3-dihydro-1H-inden-1-yl)carbamate;
(S)-quinuclidin-3-yl((R)-5-(4-butylphenyl)-6-fluoro-2,2-dimethyl-2,3-dihydro-1H-inden-1-yl)carbamate;
(S)-quinuclidin-3-yl((R)-6-fluoro-2,2-dimethyl-5-(4-propylphenyl)-2,3-dihydro-1H-inden-1-yl)carbamate;
(S)-quinuclidin-3-yl((R)-6-fluoro-5-(4-isobutylphenyl)-2,2-dimethyl-2,3-dihydro-1H-inden-1-yl)carbamate;
(S)-quinuclidin-3-yl((R)-6-fluoro-5-(4-methoxy-3,5-dimethylphenyl)-2,2-dimethyl-2,3-dihydro-1H-inden-1-yl)carbamate;
(S)-quinuclidin-3-yl((R)-5-(3,5-dimethyl-4-propoxyphenyl)-6-fluoro-2,2-dimethyl-2,3-dihydro-1H-inden-1-yl)carbamate;
(S)-quinuclidin-3-yl((R)-6-fluoro-5-(4-isopropoxy-3,5-dimethylphenyl)-2,2-dimethyl-2,3-dihydro-1H-inden-1-yl)carbamate;
(S)-quinuclidin-3-yl((R)-5-(3-chloro-4-isopropoxyphenyl)-6-fluoro-2,2-dimethyl-2,3-dihydro-1H-inden-1-yl)carbamate;
(S)-quinuclidin-3-yl((R)-6-fluoro-5-(2-fluoro-4-methoxyphenyl)-2,2-dimethyl-2,3-dihydro-1H-inden-1-yl)carbamate;
(S)-quinuclidin-3-yl((R)-6-fluoro-5-(4-isopropoxyphenyl)-2,2-dimethyl-2,3-dihydro-1H-inden-1-yl)carbamate;
(S)-quinuclidin-3-yl((R)-6-fluoro-2,2-dimethyl-5-(4-propoxyphenyl)-2,3-dihydro-1H-inden-1-yl)carbamate;
(S)-quinuclidin-3-yl((R)-5-(4-butoxyphenyl)-6-fluoro-2,2-dimethyl-2,3-dihydro-1H-inden-1-yl)carbamate;
(S)-quinuclidin-3-yl((R)-5-(3-ethylphenyl)-6-fluoro-2,2-dimethyl-2,3-dihydro-1H-inden-1-yl)carbamate;
(S)-quinuclidin-3-yl((R)-6-fluoro-2,2-dimethyl-5-(3-propylphenyl)-2,3-dihydro-1H-inden-1-yl)carbamate;
(S)-quinuclidin-3-yl((R)-6-fluoro-5-(3-isopropylphenyl)-2,2-dimethyl-2,3-dihydro-1H-inden-1-yl)carbamate;
(S)-quinuclidin-3-yl((R)-6-fluoro-5-(3-isobutylphenyl)-2,2-dimethyl-2,3-dihydro-1H-inden-1-yl)carbamate;
(S)-quinuclidin-3-yl((R)-6-fluoro-5-(3-methoxyphenyl)-2,2-dimethyl-2,3-dihydro-1H-inden-1-yl)carbamate;
(S)-quinuclidin-3-yl((R)-5-(3-ethoxyphenyl)-6-fluoro-2,2-dimethyl-2,3-dihydro-1H-inden-1-yl)carbamate;
(S)-quinuclidin-3-yl((R)-6-fluoro-2,2-dimethyl-5-(3-propoxyphenyl)-2,3-dihydro-1H-inden-1-yl)carbamate;
(S)-quinuclidin-3-yl((R)-6-fluoro-5-(3-isobutoxyphenyl)-2,2-dimethyl-2,3-dihydro-1H-inden-1-yl)carbamate;
(S)-quinuclidin-3-yl((R)-5-(3-butoxyphenyl)-6-fluoro-2,2-dimethyl-2,3-dihydro-1H-inden-1-yl)carbamate;
(S)-quinuclidin-3-yl((R)-5-(4-ethoxy-3,5-dimethylphenyl)-6-fluoro-2,2-dimethyl-2,3-dihydro-1H-inden-1-yl)carbamate;
(S)-quinuclidin-3-yl((R)-6-fluoro-5-(3-isopropoxyphenyl)-2,2-dimethyl-2,3-dihydro-1H-inden-1-yl)carbamate;
(S)-quinuclidin-3-yl((R)-6-fluoro-5-(4-methoxyphenyl)-2,2-dimethyl-2,3-dihydro-1H-inden-1-yl)carbamate;
(S)-quinuclidin-3-yl((R)-6-fluoro-5-(3-fluoro-4-isopropoxyphenyl)-2,2-dimethyl-2,3-dihydro-1H-inden-1-yl)carbamate;
(S)-quinuclidin-3-yl((R)-5-(3-chloro-4-methylphenyl)-6-fluoro-2,2-dimethyl-2,3-dihydro-1H-inden-1-yl)carbamate;
(S)-quinuclidin-3-yl((R)-5-(3-chloro-4-methoxyphenyl)-6-fluoro-2,2-dimethyl-2,3-dihydro-1H-inden-1-yl)carbamate;
(S)-quinuclidin-3-yl((R)-5-(3-chloro-4-ethoxyphenyl)-6-fluoro-2,2-dimethyl-2,3-dihydro-1H-inden-1-yl)carbamate;
(S)-quinuclidin-3-yl((R)-5-(3-chloro-4-propoxyphenyl)-6-fluoro-2,2-dimethyl-2,3-dihydro-1H-inden-1-yl)carbamate;
(S)-quinuclidin-3-yl((R)-5-(4-butoxy-3-chlorophenyl)-6-fluoro-2,2-dimethyl-2,3-dihydro-1H-inden-1-yl)carbamate;
(S)-quinuclidin-3-yl((R)-6-fluoro-5-(3-fluoro-4-methoxyphenyl)-2,2-dimethyl-2,3-dihydro-1H-inden-1-yl)carbamate;
(S)-quinuclidin-3-yl((R)-5-(4-ethoxy-3-fluorophenyl)-6-fluoro-2,2-dimethyl-2,3-dihydro-1H-inden-1-yl)carbamate;
(S)-quinuclidin-3-yl((R)-6-fluoro-5-(3-fluoro-4-propoxyphenyl)-2,2-dimethyl-2,3-dihydro-1H-inden-1-yl)carbamate;
(S)-quinuclidin-3-yl((R)-5-(2-chloro-4-isopropoxyphenyl)-6-fluoro-2,2-dimethyl-2,3-dihydro-1H-inden-1-yl)carbamate;
(S)-quinuclidin-3-yl((R)-5-(4-ethyl-3-fluorophenyl)-6-fluoro-2,2-dimethyl-2,3-dihydro-1H-inden-1-yl)carbamate;
(S)-quinuclidin-3-yl((R)-6-fluoro-5-(4-isopropoxy-3-methylphenyl)-2,2-dimethyl-2,3-dihydro-1H-inden-1-yl)carbamate;
(S)-quinuclidin-3-yl((R)-6-fluoro-5-(4-isopropoxy-3-methoxyphenyl)-2,2-dimethyl-2,3-dihydro-1H-inden-1-yl)carbamate;
(S)-quinuclidin-3-yl((R)-6-fluoro-5-(4-isopropoxy-2-methylphenyl)-2,2-dimethyl-2,3-dihydro-1H-inden-1-yl)carbamate;
(S)-quinuclidin-3-yl((R)-5-(3,5-difluoro-4-isopropoxyphenyl)-6-fluoro-2,2-dimethyl-2,3-dihydro-1H-inden-1-yl)carbamate;
(S)-quinuclidin-3-yl((R)-5-(2-chloro-4-(cyclopropylmethoxy)phenyl)-6-fluoro-2,2-dimethyl-2,3-dihydro-1H-inden-1-yl)carbamate;
(S)-quinuclidin-3-yl((R)-5-(3-chloro-4-(cyclopropylmethoxy)phenyl)-6-fluoro-2,2-dimethyl-2,3-dihydro-1H-inden-1-yl)carbamate;
(S)-quinuclidin-3-yl((R)-5-(3,5-dichloro-4-isopropoxyphenyl)-6-fluoro-2,2-dimethyl-2,3-dihydro-1H-inden-1-yl)carbamate;
(S)-quinuclidin-3-yl((R)-5-(4-(tert-butoxy)phenyl)-6-fluoro-2,2-dimethyl-2,3-dihydro-1H-inden-1-yl)carbamate;
(S)-quinuclidin-3-yl((R)-5-(4-(tert-butoxy)-2-chlorophenyl)-6-fluoro-2,2-dimethyl-2,3-dihydro-1H-inden-1-yl)carbamate;
(S)-quinuclidin-3-yl((R)-5-(2-chloro-4-methoxyphenyl)-6-fluoro-2,2-dimethyl-2,3-dihydro-1H-inden-1-yl)carbamate;
(S)-quinuclidin-3-yl((R)-5-(2-chloro-4-ethoxyphenyl)-6-fluoro-2,2-dimethyl-2,3-dihydro-1H-inden-1-yl)carbamate;
(S)-quinuclidin-3-yl((R)-5-(4-ethoxy-3-methylphenyl)-6-fluoro-2,2-dimethyl-2,3-dihydro-1H-inden-1-yl)carbamate;
(S)-quinuclidin-3-yl((R)-6-fluoro-2,2-dimethyl-5-(3-methyl-4-propoxyphenyl)-2,3-dihydro-1H-inden-1-yl)carbamate;
(S)-quinuclidin-3-yl((R)-5-(4-(difluoromethoxy)-3-methylphenyl)-6-fluoro-2,2-dimethyl-2,3-dihydro-1H-inden-1-yl)carbamate;
(S)-quinuclidin-3-yl((R)-6-fluoro-5-(3-fluoro-5-isopropoxyphenyl)-2,2-dimethyl-2,3-dihydro-1H-inden-1-yl)carbamate;
(S)-quinuclidin-3-yl((R)-6-fluoro-5-(3-fluoro-5-isobutoxyphenyl)-2,2-dimethyl-2,3-dihydro-1H-inden-1-yl)carbamate;
(S)-quinuclidin-3-yl((R)-6-fluoro-5-(3-fluoro-5-(2,2,2-trifluoroethoxy)phenyl)-2,2-dimethyl-2,3-dihydro-1H-inden-1-yl)carbamate;
(S)-quinuclidin-3-yl((R)-5-(4-chloro-3-isopropoxyphenyl)-6-fluoro-2,2-dimethyl-2,3-dihydro-1H-inden-1-yl)carbamate;
(S)-quinuclidin-3-yl((R)-5-(3-chloro-5-isobutoxyphenyl)-6-fluoro-2,2-dimethyl-2,3-dihydro-1H-inden-1-yl)carbamate;
(S)-quinuclidin-3-yl((R)-5-(3-chloro-5-isopropoxyphenyl)-6-fluoro-2,2-dimethyl-2,3-dihydro-1H-inden-1-yl)carbamate;
(S)-quinuclidin-3-yl((R)-5-(2-chloro-4-(2,2,2-trifluoroethoxy)phenyl)-6-fluoro-2,2-dimethyl-2,3-dihydro-1H-inden-1-yl)carbamate;
(S)-quinuclidin-3-yl((R)-5-(4-ethylphenyl)-2,2,6-trimethyl-2,3-dihydro-1H-inden-1-yl)carbamate;
(S)-quinuclidin-3-yl((R)-2,2,6-trimethyl-5-(4-propylphenyl)-2,3-dihydro-1H-inden-1-yl)carbamate;
(S)-quinuclidin-3-yl((R)-5-(4-isopropylphenyl)-2,2,6-trimethyl-2,3-dihydro-1H-inden-1-yl)carbamate;
(S)-quinuclidin-3-yl((R)-5-(4-butylphenyl)-2,2,6-trimethyl-2,3-dihydro-1H-inden-1-yl)carbamate;
(S)-quinuclidin-3-yl((R)-5-(4-isobutylphenyl)-2,2,6-trimethyl-2,3-dihydro-1H-inden-1-yl)carbamate;
(S)-quinuclidin-3-yl((R)-5-(4-ethoxyphenyl)-2,2,6-trimethyl-2,3-dihydro-1H-inden-1-yl)carbamate;
(S)-quinuclidin-3-yl((R)-2,2,6-trimethyl-5-(4-propoxyphenyl)-2,3-dihydro-1H-inden-1-yl)carbamate;
(S)-quinuclidin-3-yl((R)-5-(4-isopropoxyphenyl)-2,2,6-trimethyl-2,3-dihydro-1H-inden-1-yl)carbamate;
(S)-quinuclidin-3-yl((R)-5-(4-butoxyphenyl)-2,2,6-trimethyl-2,3-dihydro-1H-inden-1-yl)carbamate;
(S)-quinuclidin-3-yl((R)-5-(4-isobutoxyphenyl)-2,2,6-trimethyl-2,3-dihydro-1H-inden-1-yl)carbamate;
(S)-quinuclidin-3-yl((R)-5-(3-chloro-4-isopropoxyphenyl)-2,2,6-trimethyl-2,3-dihydro-1H-inden-1-yl)carbamate;
(S)-quinuclidin-3-yl((R)-5-(2-fluoro-4-methoxyphenyl)-2,2,6-trimethyl-2,3-dihydro-1H-inden-1-yl)carbamate;
(S)-quinuclidin-3-yl((R)-6-ethoxy-5-(4-ethylphenyl)-2,2-dimethyl-2,3-dihydro-1H-inden-1-yl)carbamate;
(S)-quinuclidin-3-yl((R)-6-ethoxy-2,2-dimethyl-5-(4-propylphenyl)-2,3-dihydro-1H-inden-1-yl)carbamate;
(S)-quinuclidin-3-yl((R)-6-ethoxy-5-(4-isopropylphenyl)-2,2-dimethyl-2,3-dihydro-1H-inden-1-yl)carbamate;
(S)-quinuclidin-3-yl((R)-5-(4-butylphenyl)-6-ethoxy-2,2-dimethyl-2,3-dihydro-1H-inden-1-yl)carbamate;
(S)-quinuclidin-3-yl((R)-6-ethoxy-5-(4-isobutylphenyl)-2,2-dimethyl-2,3-dihydro-1H-inden-1-yl)carbamate;
(S)-quinuclidin-3-yl((R)-6-ethoxy-5-(4-ethoxyphenyl)-2,2-dimethyl-2,3-dihydro-1H-inden-1-yl)carbamate;
(S)-quinuclidin-3-yl((R)-6-ethoxy-2,2-dimethyl-5-(4-propoxyphenyl)-2,3-dihydro-1H-inden-1-yl)carbamate;
(S)-quinuclidin-3-yl((R)-6-ethoxy-5-(4-isopropoxyphenyl)-2,2-dimethyl-2,3-dihydro-1H-inden-1-yl)carbamate;
(S)-quinuclidin-3-yl((R)-5-(4-butoxyphenyl)-6-ethoxy-2,2-dimethyl-2,3-dihydro-1H-inden-1-yl)carbamate;
(S)-quinuclidin-3-yl((R)-6-ethoxy-5-(4-isobutoxyphenyl)-2,2-dimethyl-2,3-dihydro-1H-inden-1-yl)carbamate;
(S)-quinuclidin-3-yl((R)-5-(3-chloro-4-isopropoxyphenyl)-6-ethoxy-2,2-dimethyl-2,3-dihydro-1H-inden-1-yl)carbamate;
(S)-quinuclidin-3-yl((R)-6-ethoxy-5-(2-fluoro-4-methoxyphenyl)-2,2-dimethyl-2,3-dihydro-1H-inden-1-yl)carbamate;
(S)-quinuclidin-3-yl((R)-6-(4-ethylphenyl)-2,2-dimethyl-1,2,3,4-tetrahydronaphthalen-1-yl)carbamate;
(S)-quinuclidin-3-yl((R)-2,2-dimethyl-6-(4-propylphenyl)-1,2,3,4-tetrahydronaphthalen-1-yl)carbamate;
(S)-quinuclidin-3-yl((R)-6-(4-isopropylphenyl)-2,2-dimethyl-1,2,3,4-tetrahydronaphthalen-1-yl)carbamate;
(S)-quinuclidin-3-yl((R)-6-(4-butylphenyl)-2,2-dimethyl-1,2,3,4-tetrahydronaphthalen-1-yl)carbamate;
(S)-quinuclidin-3-yl((R)-6-(4-ethoxyphenyl)-2,2-dimethyl-1,2,3,4-tetrahydronaphthalen-1-yl)carbamate;
(S)-quinuclidin-3-yl((R)-2,2-dimethyl-6-(4-propoxyphenyl)-1,2,3,4-tetrahydronaphthalen-1-yl)carbamate;
(S)-quinuclidin-3-yl((R)-6-(4-butoxyphenyl)-2,2-dimethyl-1,2,3,4-tetrahydronaphthalen-1-yl)carbamate;
(S)-quinuclidin-3-yl((R)-6-(4-isobutoxyphenyl)-2,2-dimethyl-1,2,3,4-tetrahydronaphthalen-1-yl)carbamate;
(S)-quinuclidin-3-yl((R)-6-(4-methoxy-3,5-dimethylphenyl)-2,2-dimethyl-1,2,3,4-tetrahydronaphthalen-1-yl)carbamate;
(S)-quinuclidin-3-yl((R)-6-(3,5-dimethyl-4-propoxyphenyl)-2,2-dimethyl-1,2,3,4-tetrahydronaphthalen-1-yl)carbamate;
(S)-quinuclidin-3-yl((R)-6-(4-isopropoxy-3,5-dimethylphenyl)-2,2-dimethyl-1,2,3,4-tetrahydronaphthalen-1-yl)carbamate;
(S)-quinuclidin-3-yl((R)-6-(3-chloro-4-isopropoxyphenyl)-2,2-dimethyl-1,2,3,4-tetrahydronaphthalen-1-yl)carbamate;
(S)-quinuclidin-3-yl((R)-6-(2-fluoro-4-methoxyphenyl)-2,2-dimethyl-1,2,3,4-tetrahydronaphthalen-1-yl)carbamate;
(S)-quinuclidin-3-yl((R)-6-(4-butoxy-3,5-dimethylphenyl)-2,2-dimethyl-1,2,3,4-tetrahydronaphthalen-1-yl)carbamate;
(S)-quinuclidin-3-yl((R)-6-(3-methoxyphenyl)-2,2-dimethyl-1,2,3,4-tetrahydronaphthalen-1-yl)carbamate;
(S)-quinuclidin-3-yl((R)-6-(3-ethoxyphenyl)-2,2-dimethyl-1,2,3,4-tetrahydronaphthalen-1-yl)carbamate;
(S)-quinuclidin-3-yl((R)-6-(3-butoxyphenyl)-2,2-dimethyl-1,2,3,4-tetrahydronaphthalen-1-yl)carbamate;
(S)-quinuclidin-3-yl((R)-2,2-dimethyl-6-(3-propylphenyl)-1,2,3,4-tetrahydronaphthalen-1-yl)carbamate;
(S)-quinuclidin-3-yl((R)-6-(3-isobutylphenyl)-2,2-dimethyl-1,2,3,4-tetrahydronaphthalen-1-yl)carbamate;
(S)-quinuclidin-3-yl((R)-6-(3-ethylphenyl)-2,2-dimethyl-1,2,3,4-tetrahydronaphthalen-1-yl)carbamate;
(S)-quinuclidin-3-yl((R)-2,2-dimethyl-6-(3-propoxyphenyl)-1,2,3,4-tetrahydronaphthalen-1-yl)carbamate;
(S)-quinuclidin-3-yl((R)-6-(4-isobutylphenyl)-2,2-dimethyl-1,2,3,4-tetrahydronaphthalen-1-yl)carbamate;
(S)-quinuclidin-3-yl((R)-6-(3-fluoro-4-isopropoxyphenyl)-2,2-dimethyl-1,2,3,4-tetrahydronaphthalen-1-yl)carbamate;
(S)-quinuclidin-3-yl((R)-6-(3-isopropylphenyl)-2,2-dimethyl-1,2,3,4-tetrahydronaphthalen-1-yl)carbamate;
(S)-quinuclidin-3-yl((R)-6-(3-isobutoxyphenyl)-2,2-dimethyl-1,2,3,4-tetrahydronaphthalen-1-yl)carbamate;
(S)-quinuclidin-3-yl((R)-6-(4-ethoxy-3,5-dimethylphenyl)-2,2-dimethyl-1,2,3,4-tetrahydronaphthalen-1-yl)carbamate;
(S)-quinuclidin-3-yl((R)-6-(3-isopropoxyphenyl)-2,2-dimethyl-1,2,3,4-tetrahydronaphthalen-1-yl)carbamate;
(S)-quinuclidin-3-yl((R)-6-(4-methoxyphenyl)-2,2-dimethyl-1,2,3,4-tetrahydronaphthalen-1-yl)carbamate;
(S)-quinuclidin-3-yl((R)-6-(4-isopropoxyphenyl)-2,2-dimethyl-1,2,3,4-tetrahydronaphthalen-1-yl)carbamate;
(S)-quinuclidin-3-yl((R)-6-(2-chloro-4-isopropoxyphenyl)-2,2-dimethyl-1,2,3,4-tetrahydronaphthalen-1-yl)carbamate;
(S)-quinuclidin-3-yl((R)-6-(4-ethyl-3-fluorophenyl)-2,2-dimethyl-1,2,3,4-tetrahydronaphthalen-1-yl)carbamate;
(S)-quinuclidin-3-yl((R)-6-(4-ethylphenyl)-7-fluoro-2,2-dimethyl-1,2,3,4-tetrahydronaphthalen-1-yl)carbamate;
(S)-quinuclidin-3-yl((R)-7-fluoro-2,2-dimethyl-6-(4-propylphenyl)-1,2,3,4-tetrahydronaphthalen-1-yl)carbamate;
(S)-quinuclidin-3-yl((R)-7-fluoro-6-(4-isopropylphenyl)-2,2-dimethyl-1,2,3,4-tetrahydronaphthalen-1-yl)carbamate;
(S)-quinuclidin-3-yl((R)-6-(4-butylphenyl)-7-fluoro-2,2-dimethyl-1,2,3,4-tetrahydronaphthalen-1-yl)carbamate;
(S)-quinuclidin-3-yl((R)-7-fluoro-6-(4-isobutylphenyl)-2,2-dimethyl-1,2,3,4-tetrahydronaphthalen-1-yl)carbamate;
(S)-quinuclidin-3-yl((R)-7-fluoro-6-(4-methoxyphenyl)-2,2-dimethyl-1,2,3,4-tetrahydronaphthalen-1-yl)carbamate;
(S)-quinuclidin-3-yl((R)-6-(4-ethoxyphenyl)-7-fluoro-2,2-dimethyl-1,2,3,4-tetrahydronaphthalen-1-yl)carbamate;
(S)-quinuclidin-3-yl((R)-7-fluoro-2,2-dimethyl-6-(4-propoxyphenyl)-1,2,3,4-tetrahydronaphthalen-1-yl)carbamate;
(S)-quinuclidin-3-yl((R)-6-(4-butoxyphenyl)-7-fluoro-2,2-dimethyl-1,2,3,4-tetrahydronaphthalen-1-yl)carbamate;
(S)-quinuclidin-3-yl((R)-7-fluoro-6-(4-isobutoxyphenyl)-2,2-dimethyl-1,2,3,4-tetrahydronaphthalen-1-yl)carbamate;
(S)-quinuclidin-3-yl((R)-7-fluoro-6-(4-methoxy-3,5-dimethylphenyl)-2,2-dimethyl-1,2,3,4-tetrahydronaphthalen-1-yl)carbamate;
(S)-quinuclidin-3-yl((R)-6-(3,5-dimethyl-4-propoxyphenyl)-7-fluoro-2,2-dimethyl-1,2,3,4-tetrahydronaphthalen-1-yl)carbamate;
(S)-quinuclidin-3-yl((R)-7-fluoro-6-(4-isopropoxy-3,5-dimethylphenyl)-2,2-dimethyl-1,2,3,4-tetrahydronaphthalen-1-yl)carbamate;
(S)-quinuclidin-3-yl((R)-6-(4-butoxy-3,5-dimethylphenyl)-7-fluoro-2,2-dimethyl-1,2,3,4-tetrahydronaphthalen-1-yl)carbamate;
(S)-quinuclidin-3-yl((R)-7-fluoro-6-(3-methoxyphenyl)-2,2-dimethyl-1,2,3,4-tetrahydronaphthalen-1-yl)carbamate;
(S)-quinuclidin-3-yl((R)-6-(3-ethoxyphenyl)-7-fluoro-2,2-dimethyl-1,2,3,4-tetrahydronaphthalen-1-yl)carbamate;
(S)-quinuclidin-3-yl((R)-6-(3-butoxyphenyl)-7-fluoro-2,2-dimethyl-1,2,3,4-tetrahydronaphthalen-1-yl)carbamate;
(S)-quinuclidin-3-yl((R)-6-(3-ethylphenyl)-7-fluoro-2,2-dimethyl-1,2,3,4-tetrahydronaphthalen-1-yl)carbamate;
(S)-quinuclidin-3-yl((R)-7-fluoro-2,2-dimethyl-6-(3-propylphenyl)-1,2,3,4-tetrahydronaphthalen-1-yl)carbamate;
(S)-quinuclidin-3-yl((R)-7-fluoro-6-(3-isobutylphenyl)-2,2-dimethyl-1,2,3,4-tetrahydronaphthalen-1-yl)carbamate;
(S)-quinuclidin-3-yl((R)-7-fluoro-6-(3-isopropylphenyl)-2,2-dimethyl-1,2,3,4-tetrahydronaphthalen-1-yl)carbamate;
(S)-quinuclidin-3-yl((R)-7-fluoro-6-(3-isobutoxyphenyl)-2,2-dimethyl-1,2,3,4-tetrahydronaphthalen-1-yl)carbamate;
(S)-quinuclidin-3-yl((R)-7-fluoro-2,2-dimethyl-6-(3-propoxyphenyl)-1,2,3,4-tetrahydronaphthalen-1-yl)carbamate;
(S)-quinuclidin-3-yl((R)-6-(3-chloro-4-isopropoxyphenyl)-7-fluoro-2,2-dimethyl-1,2,3,4-tetrahydronaphthalen-1-yl)carbamate;
(S)-quinuclidin-3-yl((R)-7-fluoro-6-(2-fluoro-4-methoxyphenyl)-2,2-dimethyl-1,2,3,4-tetrahydronaphthalen-1-yl)carbamate;
(S)-quinuclidin-3-yl((R)-6-(4-ethoxy-3,5-dimethylphenyl)-7-fluoro-2,2-dimethyl-1,2,3,4-tetrahydronaphthalen-1-yl)carbamate;
(S)-quinuclidin-3-yl((R)-7-fluoro-6-(3-isopropoxyphenyl)-2,2-dimethyl-1,2,3,4-tetrahydronaphthalen-1-yl)carbamate;
(S)-quinuclidin-3-yl((R)-7-fluoro-6-(4-isopropoxyphenyl)-2,2-dimethyl-1,2,3,4-tetrahydronaphthalen-1-yl)carbamate;
(S)-quinuclidin-3-yl((R)-7-fluoro-6-(3-fluoro-4-isopropoxyphenyl)-2,2-dimethyl-1,2,3,4-tetrahydronaphthalen-1-yl)carbamate; and
(S)-quinuclidin-3-yl((R)-6-(2-chloro-4-isopropoxyphenyl)-7-fluoro-2,2-dimethyl-1,2,3,4-tetrahydronaphthalen-1-yl)carbamate.

13. A pharmaceutical composition for inhibiting glucosylceramide synthase comprising the compound or pharmaceutically acceptable salt thereof as claimed in claim 1 as an active ingredient.

14. A pharmaceutical composition as claimed in claim 13, for preventing or treating Gaucher disease, Fabry disease, Tay-Sachs disease, or Parkinson's disease.

15. A method for inhibiting glucosylceramide synthase in a mammal, which comprises administering a therapeutically effective amount of the compound or pharmaceutically acceptable salt thereof as claimed in claim 1 to the mammal in need thereof.
